(19) United States
(12) Patent Application Publication
(10) Pub. No.: US 2026/0124213 A1
(43) Pub. Date: May 7, 2026

(54) STAPHYLOCOCCUS AUREUS PBP4 INHIBITORS AND METHOD OF USE

(71) Applicants: UNIVERSITY OF ROCHESTER, Rochester, NY (US); UNIVERSITY OF NOTRE DAME DU LAC, South Bend, IN (US)

(72) Inventors: Paul M. DUNMAN, Pittsford, NY (US); Christian MELANDER, South Bend, IN (US)

(21) Appl. No.: 19/117,956

(22) PCT Filed: Oct. 4, 2023
(86) PCT No.: PCT/US2023/075996

§ 371 Date: Apr. 2, 2025

Related U.S. Application Data
(60) Provisional application No. 63/378,335, filed on Oct. 4, 2022.

(51) Int. Cl. A61K 31/5377; A61K 31/17; A61K 31/40; A61K 31/4184; A61K 31/45; A61K 31/454; A61K 45/06; A61P 31/04; C07C 275/40; C07D 211/16; C07D 211/68; C07D 295/155; C07D 405/12; C07D 405/14; C07D 409/04; C07D 409/14; C07D 417/12

(52) U.S. Cl. CPC A61K 31/5377; A61K 31/17; A61K 31/40; A61K 31/4184; A61K 31/45; A61K 31/454; A61K 45/06; A61P 31/04; C07C 275/40; C07D 211/16; C07D 211/68; C07D 295/155; C07D 405/12; C07D 405/14; C07D 409/04; C07D 409/14; C07D 417/12

(57) ABSTRACT

Described are compounds, compositions, and method for treating Penicillin Binding Protein 4 (PBP4) related diseases and/or disorders, such as bacterial infections.

### CROSS-REFERENCE TO RELATED APPLICATION(S)

This application claims priority to U.S. Provisional Patent Application No. 63/378,335, filed on Oct. 4, 2022, the entire contents of which are fully incorporated herein by reference.

### STATEMENT OF GOVERNMENT INTEREST

This invention was made with government support under grants AR072000, AI134685, AR069655, AR081050, and DE022350 awarded by the National Institutes of Health. The government has certain rights in this invention. The government has certain rights in this invention.

### TECHNICAL FIELD

The present disclosure relates to compounds, compositions, and methods for treating Penicillin Binding Protein 4 (PBP4) related diseases and/or disorders, such as bacterial infections.

### INTRODUCTION

Penicillin Binding Protein 4 (PBP4), which is a sub-type of the penicillin binding protein family, is an enzyme that participates in the production of peptidoglycan, the major component of cell walls in bacteria. PBP4 is overexpressed in antibiotic resistance strains and inactivation of PBP4 activity decreases bacterial migration.

### SUMMARY

In some aspects, the present disclosure provides compounds of formula (I), or a pharmaceutically acceptable salt thereof, and a pharmaceutically acceptable carrier,

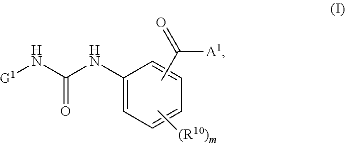

wherein:
A¹ is

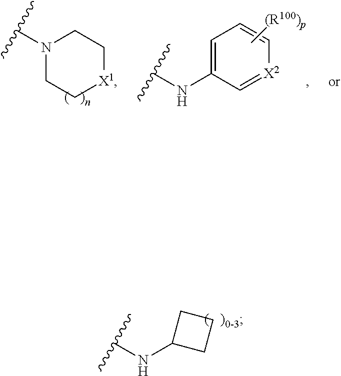

G¹ is a 6- to 12-membered aryl, C₃₋₁₀carbocyclyl, a 5- to 12-membered heteroaryl, or a 4- to 12-membered heterocyclyl, wherein G¹ is optionally substituted with 1-3 R¹;
X¹ is CHR^{X}, C═O, or O;
X² is CH or N;
R^{X}, at each occurrence, is C₁₋₆alkyl, hydrogen, C₁₋₄haloalkyl, halogen, or cyano;
R¹, at each occurrence, is independently halogen, cyano, C₁₋₆alkyl, C₁₋₆haloalkyl, —OR^{1a}, —SR^{1a}, —CO₂R^{1a}, —C(O)R^{1a}, —SO₂R^{1b}, —N(R^{1b})₂, —CO₂N(R^{1b})₂, —NO₂, G^{1a}, —OG^{1a}, —SG^{1a}, N(R^{1b})-G^{1b}, -L¹-Y¹, —O-L¹-Y¹, —S-L¹-Y¹, or N(R^{1b})-L¹-Y¹;
L¹, at each occurrence, is independently a C₁₋₆alkylene, wherein optionally 1 or 2 methylene groups in the alkylene of L¹ are independently replaced with —O—, —S—, —SO₂—, —C(O)—, or —N(R^{1b})—, wherein 2 methylene groups replaced with —O—, —S—, —SO₂—, or —N(R^{1b})— are separated by two or more carbon atoms in the alkylene,
Y¹, at each occurrence, is independently hydrogen, halogen, cyano, C₁₋₆alkyl, C₁₋₆haloalkyl, —OR^{1a}, —SR^{1a}, —CO₂R^{1a}, —C(O)R^{1a}, —SO₂R^{1b}, —N(R^{1b})₂, —CO₂N(R^{1b})₂, G^{1a}, or —OG^{1a};
R^{1a}, at each occurrence, is independently hydrogen, C₁₋₆alkyl, or C₁₋₂haloalkyl;
R^{1b}, at each occurrence, is independently hydrogen or C₁₋₆alkyl;
G^{1a}, at each occurrence, is independently C₃₋₆carbocyclyl, phenyl, a 4- to 6-membered heterocyclyl, or a 5- to 6-membered heteroaryl, wherein G^{1a} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
G^{1b}, at each occurrence, is independently C₃₋₆carbocyclyl or phenyl, wherein G^{1b} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
R¹⁰, at each occurrence, is independently halogen, C₁₋₄alkyl, —OC₁₋₄alkyl, C₁₋₂haloalkyl, cyano, or —OC₁₋₂haloalkyl;
R¹⁰⁰, at each occurrence, is independently halogen, C₁₋₄alkyl, —OC₁₋₄alkyl, C₁₋₂haloalkyl, cyano, or —OC₁₋₂haloalkyl;
m is 0, 1, 2, 3, or 4;
n is 0, 1, or 2; and
p is 0, 1, 2, or 3.

In other aspects, the present disclosure provides compounds selected from the group consisting of:

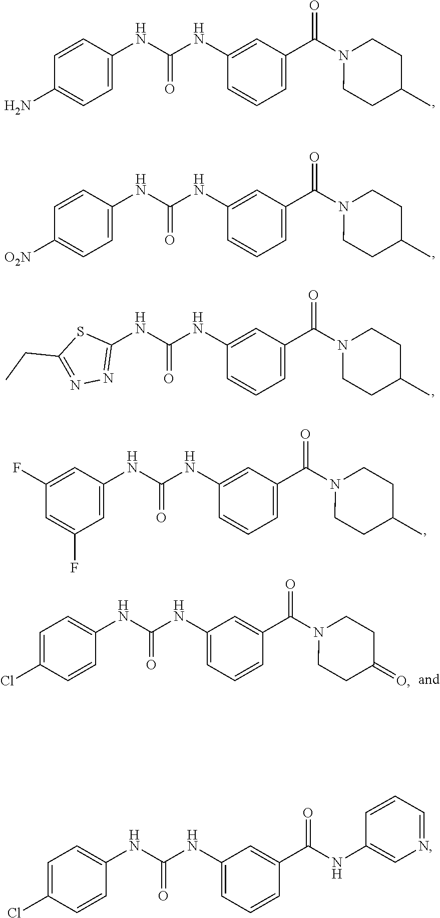

or a pharmaceutically acceptable salt thereof.

In other aspects, the present disclosure provides pharmaceutical compositions comprising a compound of formula (II), or a pharmaceutically acceptable salt thereof, and a pharmaceutically acceptable carrier,

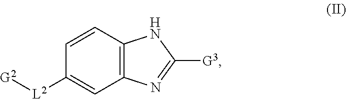

wherein:
G² is a 5- to 12-membered heteroaryl, a 6- to 12-membered aryl, C₃₋₁₀carbocyclyl, or a 4- to 12-membered heterocyclyl, wherein G² is optionally substituted with 1-3 R²;
L² is —(CH₂)₀₋₄—C(O)—(CH₂)₀₋₄—N(H)—;
G³ is a 5- to 12-membered heteroaryl, a 6- to 12-membered aryl, C₃₋₁₀carbocyclyl, or a 4- to 12-membered heterocyclyl, wherein G³ is optionally substituted with 1-3 R³;
R², at each occurrence, is independently halogen, cyano, C₁₋₆alkyl, C₁₋₆haloalkyl, —OR^{2a}, —SR^{2a}, —CO₂R^{2a}, —C(O)R^{2a}, —SO₂R^{2b}, —N(R^{2b})₂, —CO₂N(R^{2b})₂, —NO₂, G^{2a}, —OG^{2a}, —SG^{2a}, or —N(R^{2b})-G^{2b};
R³, at each occurrence, is independently halogen, cyano, C₁₋₆alkyl, C₁₋₆haloalkyl, —OR^{3a}, —SR^{3a}, —CO₂R^{3a}, —C(O)R^{3a}, —SO₂R^{3b}, —N(R^{3b})₂, —CO₂N(R^{3b})₂, —NO₂, G^{3a}, —OG^{3a}, —SG^{3a}, or —N(R^{2b})-G^{2b};
R^{2a} and R^{3a}, at each occurrence, are each independently hydrogen or C₁₋₆alkyl;
R^{2b} and R^{3b}, at each occurrence, are each independently hydrogen or C₁₋₆alkyl;
G^{2a} and G^{3a}, at each occurrence, are each independently C₃₋₆carbocyclyl, phenyl, a 4- to 6-membered heterocyclyl, or a 5- to 6-membered heteroaryl, wherein G^{2a} and G^{3a} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl; and
G^{2b} and G^{3b}, at each occurrence, are each independently C₃₋₆carbocyclyl or phenyl, wherein G^{2b} and G^{3b} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl.

In other aspects, the present disclosure provides compounds selected from the group consisting of:

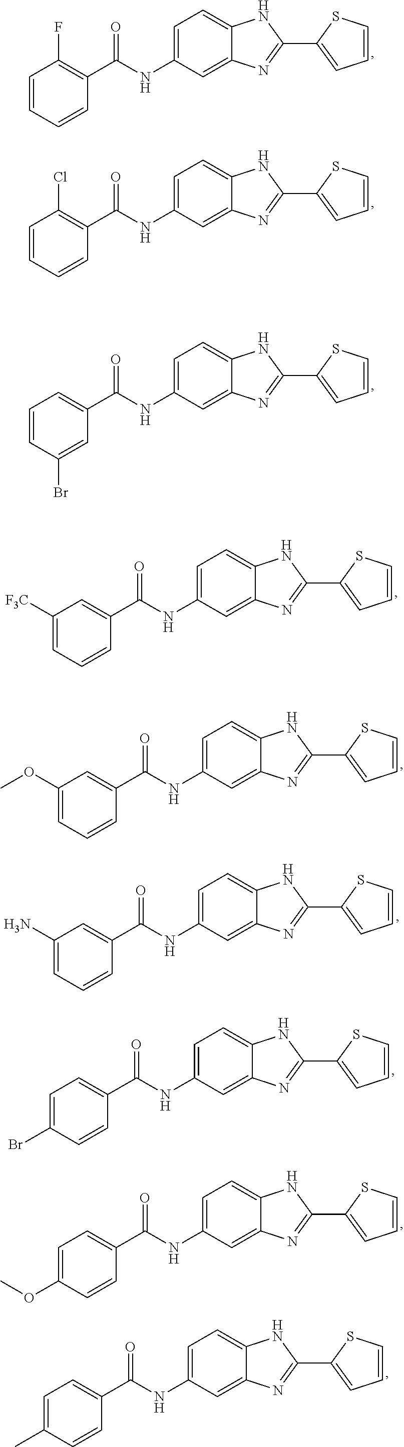

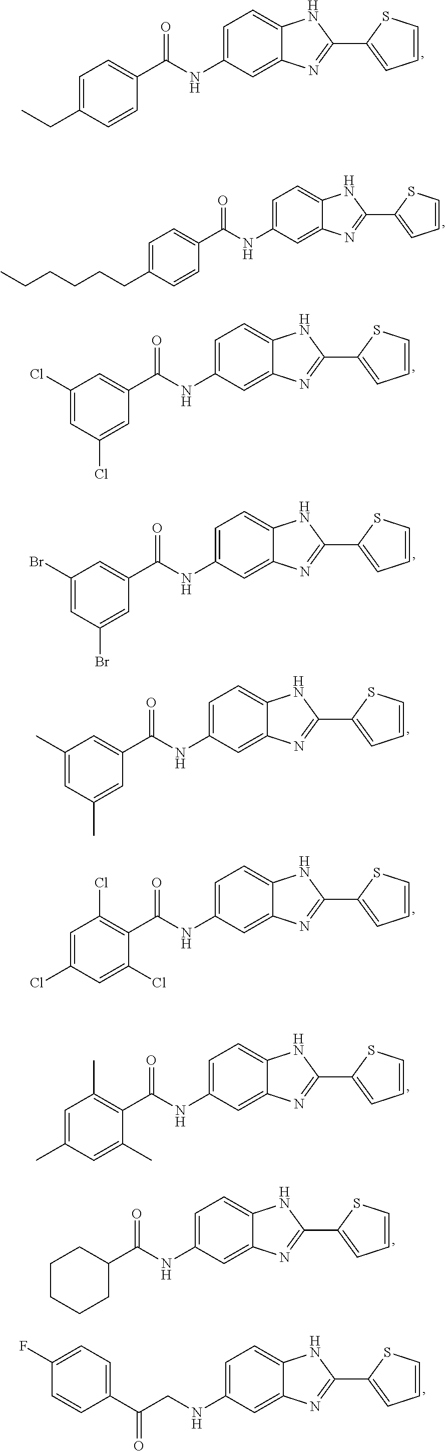

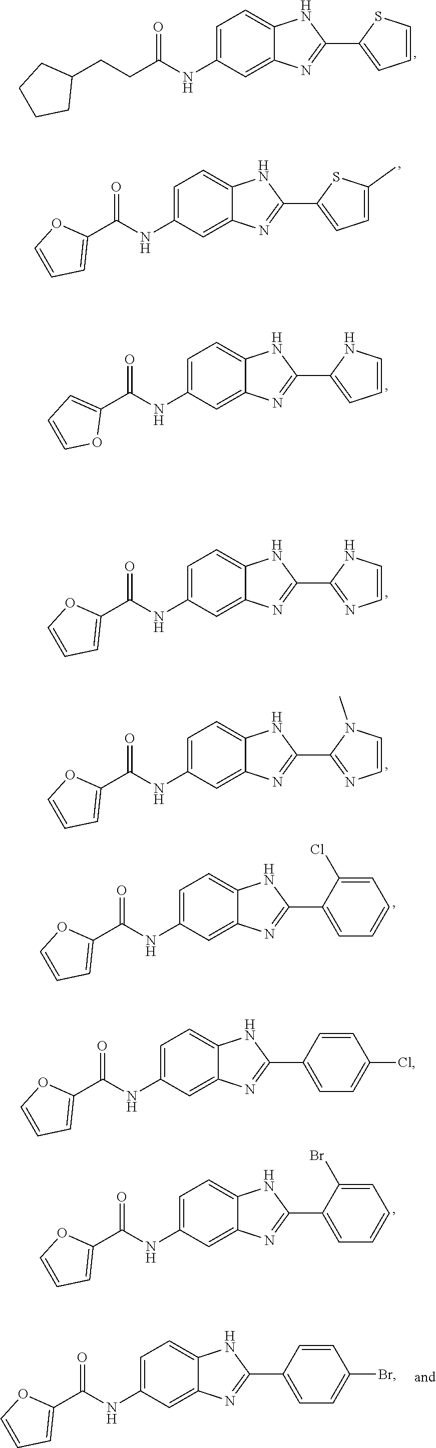

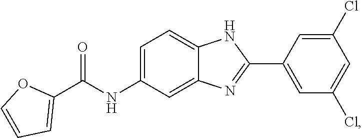 [SMILES: O=C(Nc1ccc2[nH]c(-c3cc(Cl)cc(Cl)c3)nc2c1)c1ccco1]

or a pharmaceutically acceptable salt thereof.

The pharmaceutical compositions provided in the present disclosure may further comprise an antibiotic. The antibiotic may comprise one or more selected from the group consisting of a cephalosporin, a carbapenem, an aminoglycoside, a fluoroquinolone, a glycopeptide, a lipoglycopeptide, a macrolide, a monobactams, an oxazolidinone, a penicillin, a polypeptide, a rifamycin, a sulfonamide, a streptogramin, and a tetracycline.

In other aspects, the present disclosure provides methods for treating a disease or disorder associated with a bacterial infection in a mammal, the method comprising administering to the mammal a therapeutically effective amount of the compound of formula (I) or (II), or pharmaceutically acceptable salt thereof, or the pharmaceutical composition.

The disease or disorder may be associated with a Staphylococcus infection, penicillin binding protein 4 dysfunction, or a combination thereof. The Staphylococcus infection may be the result of a methicillin resistant Staphylococcus aureus strain. The methicillin resistant Staphylococcus aureus strain may be AH-1263, ATCC BAA-1556, AH-2204, BAA-811, 700789, BAA-1770, 43300, BAA-168, 33591, BAA-44, 700699, or a combination thereof.

In other aspects of the present disclosure, the compounds, pharmaceutically acceptable salts thereof, and pharmaceutical compositions, described herein, may be used in the treatment of a disease or disorder associated with a bacterial infection in a mammal.

In other aspects of the present disclosure, the compounds, pharmaceutically acceptable salts thereof, and pharmaceutical compositions, described herein, may be used for the preparation of a medicament for the treatment of a disease or disorder associated with a bacterial infection in a mammal.

### BRIEF DESCRIPTION OF THE DRAWINGS

FIG. 1A graphically shows the Z′ factor distribution of the developed screening paradigm performed in wild-type methicillin resistant Staphylococcus aureus (MRSA) strain USA 300 and in a genetically engineered USA 300 line lacking functional penicillin binding protein 4 (Δpbp4).

FIG. 1B graphically displays the cytotoxicity of compounds 5784306, 7611906, 7729837, 9009498, 7974147, and 9314848 in HepG2 cells at various concentrations ranging from 50-400 μM.

FIG. 2A graphically shows the fold change of gene expression in MRSA strain USA 300 treated with compounds 5784306, 7611906, 7729837, 9009498, 7974147, and 9314848.

FIG. 2B graphically shows the fold change of gene expression in MRSA strain CRB treated with compounds 5784306, 7611906, 7729837, 9009498, 7974147, and 9314848.

FIG. 3A shows the chemical structures for compounds 772983 and 9314848.

FIG. 3B graphically displays the impact of Triton X-100 in wild-type and MRSA stain USA 300 cells and USA300 cells lacking functional penicillin binding protein 4 (Δpbp4) in the presence of compound 9314848.

FIG. 4A graphically shows the displacement of ampicillin (“amp”), kanamycin (“kan”), and compound 9314848 against Bocillin™ for wild-type penicillin binding protein 4 (PBP4) as measured by fluorescence polarization.

FIG. 4B graphically displays the dose-response curves binding curves for ampicillin, kanamycin, and compound 9314848 as determined with fluorescence polarization.

FIG. 4C shows the image of a gel after incubating PBP4 with fluorescently labeled Bocillin™ and challenging with various concentrations of cefoxitin and a high concentration of kanamycin.

FIG. 4D shows the image of a gel after incubating PBP4 with fluorescently labeled Bocillin™ and challenging with various concentrations of compound 9314848.

FIG. 4E graphically shows the dose-response binding curve for Bocillin™ after pre-incubation with cefoxitin, kanamycin, and compound 9314848.

FIG. 5 graphically shows the results to the μSiM-CA canaliculi model experiments with MRSA USA 300 strain treated with compound 9314848.

FIG. 6 shows the compounds synthesized from the compound 9314848 scaffold.

FIG. 7A graphically shows the migration of the MRSA USA 300 strain treated with compounds 1, 4, and 6 in at the top of a μSiM-CA device that mimics the orifices of osteocyte lacuna-canalicular networks.

FIG. 7B graphically shows the migration of the MRSA USA 300 strain treated with compounds 1, 4, and 6 in at the bottom of a μSiM-CA device that mimics the orifices of osteocyte lacuna-canalicular networks.

FIG. 8 shows the compounds synthesized from the compound 7729837 scaffold.

FIG. 9A graphically displays the time-kill curves for compound 4, which was developed from the compound 7729837 scaffold.

FIG. 9B graphically displays the time-kill curves for compound 8, which was developed from the compound 7729837 scaffold.

### DETAILED DESCRIPTIONS

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various way.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein. The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

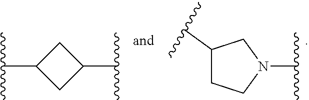 [SMILES: *C1CC(*)C1 ; *C1CCN(*)C1]

Cycloalkylene and heterocyclylene include a geminal divalent groups such as 1,1-C₃₋₆cycloalkylene

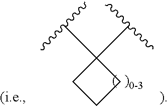

A further example is 1,1-cyclopropylen

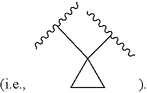 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

### Compounds

Compounds of the present disclosure are set forth in the following numbered embodiments. The first embodiment is denoted E1, another embodiment is denoted E2 and so forth.

E1. A compound of formula (I):

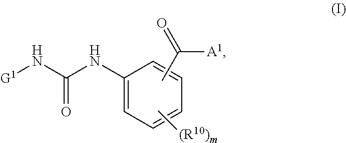

wherein:
A¹ is

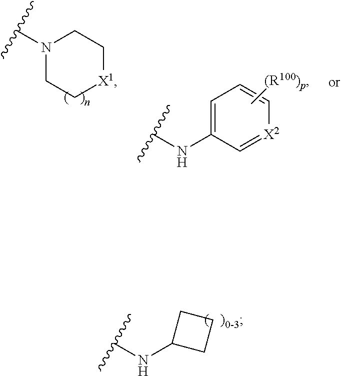

G¹ is a 6- to 12-membered aryl, C₃₋₁₀carbocyclyl, a 5- to 12-membered heteroaryl, or a 4- to 12-membered heterocyclyl, wherein G¹ is optionally substituted with 1-3 R¹;
X¹ is CHR^{X}, C═O, or O;
X² is CH or N;
R^{X}, at each occurrence, is C₁₋₆alkyl, hydrogen, C₁₋₄haloalkyl, halogen, or cyano;
R¹, at each occurrence, is independently halogen, cyano, C₁₋₆alkyl, C₁₋₆haloalkyl, —OR^{1a}, —SR^{1a}, —CO₂R^{1a}, —C(O)R^{1a}, —SO₂Rb, —N(R^{1b})₂, —CO₂N(R^{1b})₂, —NO₂, G^{1a}, —OG^{1a}, —SG^{1a}, —N(R^{1b})-G^{1b}, -L¹-Y¹, —O-L¹-Y¹, —S-L¹-Y¹, or —N(R^{1b})-L¹-Y¹;
L¹, at each occurrence, is independently a C₁₋₆alkylene, wherein optionally 1 or 2 methylene groups in the alkylene of L¹ are independently replaced with —O—, —S—, —SO₂—, —C(O)—, or —N(R^{1b})—, wherein 2 methylene groups replaced with —O—, —S—, —SO₂—, or —N(R^{1b})— are separated by two or more carbon atoms in the alkylene;
Y¹, at each occurrence, is independently hydrogen, halogen, cyano, C₁₋₆alkyl, C₁₋₆haloalkyl, —OR^{1a}, —SR^{1a}, —C₀₂R^{1a}, —C(O)R^{1a}, —SO₂R^{1b}, —N(R^{1b})₂, —CO₂N(R^{1b})₂, G^{1a}, or —OG^{1a};
R^{1a}, at each occurrence, is independently hydrogen, C₁₋₆alkyl, or C₁₋₂haloalkyl;
R^{1b}, at each occurrence, is independently hydrogen or C₁₋₆alkyl;
G^{1a}, at each occurrence, is independently C₃₋₆carbocyclyl, phenyl, a 4- to 6-membered heterocyclyl, or a 5- to 6-membered heteroaryl, wherein G^{1a} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
G^{1b}, at each occurrence, is independently C₃₋₆carbocyclyl or phenyl, wherein G^{1b} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
R¹⁰, at each occurrence, is independently halogen, C₁₋₄alkyl, —OC₁₋₄alkyl, C₁₋₂haloalkyl, cyano, or —OC₁₋₂haloalkyl;
R¹⁰⁰, at each occurrence, is independently halogen, C₁₋₄alkyl, —OC₁₋₄alkyl, C₁₋₂haloalkyl, cyano, or —OC₁₋₂haloalkyl;
n is 0, 1, 2, 3, or 4;
n is 0, 1, or 2; and
p is 0, 1, 2, or 3.

E2. The compound of E1, or a pharmaceutically acceptable salt thereof, wherein the compound of formula (I) is a compound of formula (I-a):

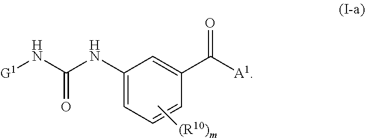

E3. The compound of E1 or E2, or a pharmaceutically acceptable salt thereof, wherein G¹ is the 6- to 12-membered aryl.

E4. The compound of any one of E1-E3, or a pharmaceutically acceptable salt thereof, wherein the 6- to 12-membered aryl at G¹ is phenyl.

E5. The compound of any one of E1-E4, or a pharmaceutically acceptable salt thereof, wherein G¹ is substituted with 1-2 substituents selected from the group consisting of halogen, C₁₋₆alkyl, —OC₁₋₄alkyl, —NH₂, —OC₁₋₂fluoroalkyl, C₁₋₂fluoroalkyl, or —NO₂.

E6. The compound of any one of E1-E5, or a pharmaceutically acceptable salt thereof, wherein G¹ is

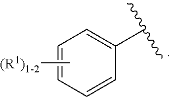

E7. The compound of any one of E1-E6, or a pharmaceutically acceptable salt thereof, wherein n is 0.

E8. The compound of any one of E1-E7, or a pharmaceutically acceptable salt thereof, wherein A¹ is

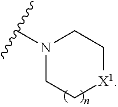

E9. The compound of any one of E1-E8, or a pharmaceutically acceptable salt thereof, wherein n is 1.

E10. The compound of E9, or a pharmaceutically acceptable salt thereof, wherein X¹ is CHR^{X}.

E11. The compound of any one of E1-E10, or a pharmaceutically acceptable salt thereof, wherein R^{X} is C₁₋₆alkyl.

E12. The compound of any one of E1-E11, or a pharmaceutically acceptable salt thereof, wherein R^{X} is methyl.

E13. The compound of any one of E1-E8, or a pharmaceutically acceptable salt thereof, wherein X¹ is C═O.

E14. The compound of any one of E1-E8, or a pharmaceutically acceptable salt thereof, wherein X is O.

E15. The compound of any one of E1-E14, or a pharmaceutically acceptable salt thereof, wherein A¹ is H

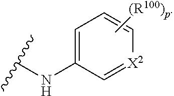

E16. The compound of any one of E1-E15, or a pharmaceutically acceptable salt thereof, wherein X² is CH.

E17. The compound ofany one of E1-E15, or a pharmaceutically acceptable salt thereof, wherein p is 1 or 2.

E18. The compound of any one of E1-E15, or a pharmaceutically acceptable salt thereof, wherein R¹⁰⁰ is F, C₁, —CH₃, or —OCH₃.

E19. The compound of any one of E1-E15, or a pharmaceutically acceptable salt thereof, wherein X² is N.

E20. The compound of any one of E1-E15, or a pharmaceutically acceptable salt thereof, wherein the compound of formula (I) is selected from the group consisting of:

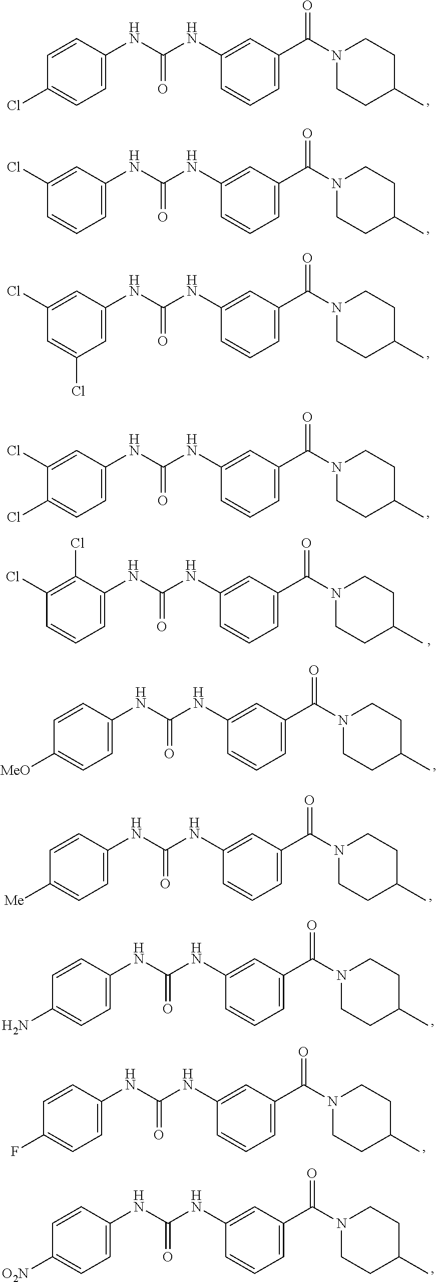

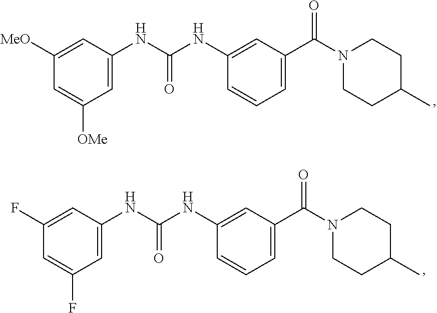 [SMILES: COc1cc(NC(=O)Nc2cccc(C(=O)N3CCC(C)CC3)c2)cc(OC)c1 ; CC1CCN(C(=O)c2cccc(NC(=O)Nc3cc(F)cc(F)c3)c2)CC1]

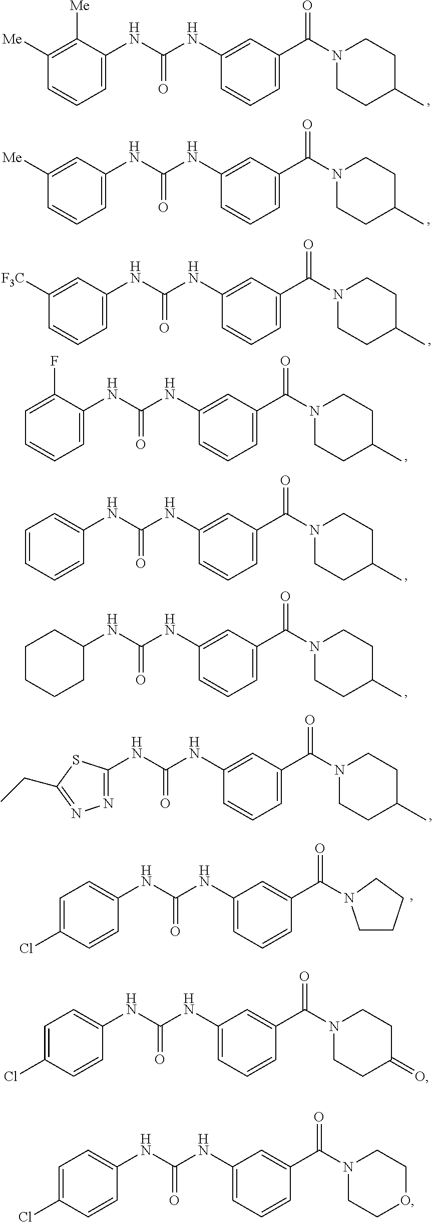

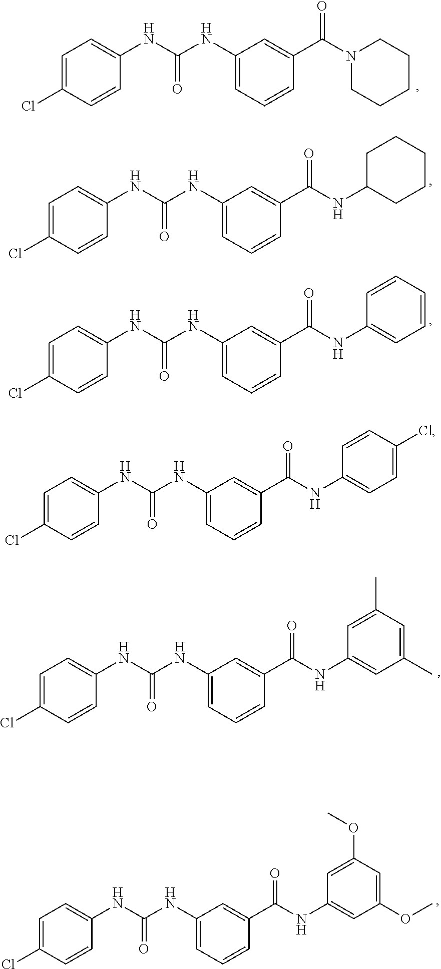

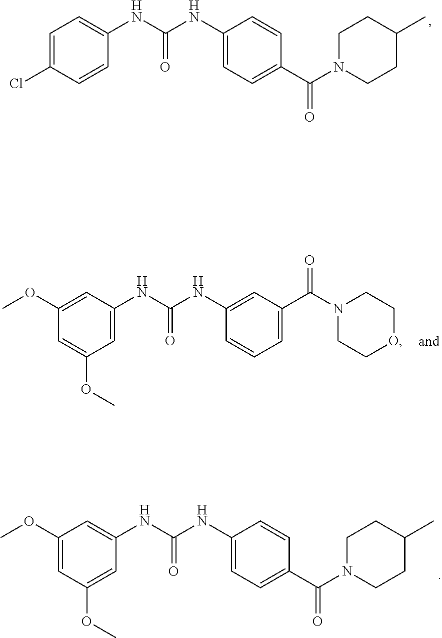

E21. A compound, or a pharmaceutically acceptable salt thereof, wherein the compound is selected from the group consisting of:

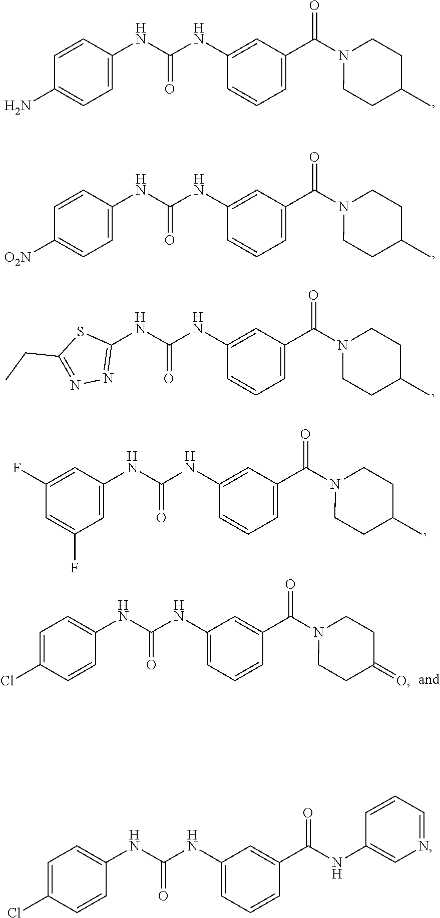

E22. A compound of formula (II), or a pharmaceutically acceptable salt thereof,

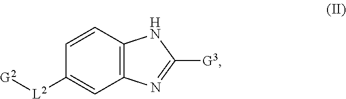

wherein:
G² is a 5- to 12-membered heteroaryl, a 6- to 12-membered aryl, C₃₋₁₀carbocyclyl, or a 4- to 12-membered heterocyclyl, wherein G² is optionally substituted with 1-3 R²;
L² is —(CH₂)₀₋₄—C(O)—(CH₂)₀₋₄—N(H)—;
G³ is a 5- to 12-membered heteroaryl, a 6- to 12-membered aryl, C₃₋₁₀carbocyclyl, or a 4- to 12-membered heterocyclyl, wherein G³ is optionally substituted with 1-3 R³;
R², at each occurrence, is independently halogen, cyano, C₁₋₆alkyl, C₁₋₆haloalkyl, —OR², —SR^{2a}, —CO₂R^{2a}, —C(O)R^{2a}, —SO₂R^{2b}, —N(R^{2b})₂, —CO₂N(R^{2b})₂, —NO₂, G^{2a}, —OG^{2a}, —SG^{2a}, or —N(R^{2b})-G^{2b};
R³, at each occurrence, is independently halogen, cyano, C₁₋₆alkyl, C₁₋₆haloalkyl, —OR^{3a}, —SR^{3a}, —CO₂R^{3a}, —C(O)R^{3a}, —SO₂R^{3b}, —N(R^{3b})₂, —CO₂N(R^{3b})₂, —NO₂, G^{3a}, —OG^{3a}, —SG^{3a}, or —N(R^{2b})-G^{2b};
R^{2a} and R^{3a}, at each occurrence, are each independently hydrogen or C₁₋₆alkyl;
R^{2b} and R^{3b}, at each occurrence, are each independently hydrogen or C₁₋₆alkyl;
G^{2a} and G^{3a}, at each occurrence, are each independently C₃₋₆carbocyclyl, phenyl, a 4- to 6-membered heterocyclyl, or a 5- to 6-membered heteroaryl, wherein G^{2a} and G^{3a} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
   and
G^{2b} and G^{3b}, at each occurrence, are each independently C₃₋₆carbocyclyl or phenyl, wherein G^{2b} and G^{3b} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl.

E23. The compound of E22, or a pharmaceutically acceptable salt thereof, wherein L² is —N(H)—C(O)—.

E24. The compound of E22, or a pharmaceutically acceptable salt thereof, wherein L² is —C(O)—(CH₂)₁₋₃—N(H)—.

E25. The compound of E22, or a pharmaceutically acceptable salt thereof, wherein L² is —(CH₂)₁₋₃—C(O)—N(H)—.

E26. The compound of any one of E22-E25, or a pharmaceutically acceptable salt thereof, wherein G² is the 6- to 12-membered aryl.

E27. The compound E26, or a pharmaceutically acceptable salt thereof, where the 6- to 12-membered aryl at G² is phenyl.

E28. The compound of E27, or a pharmaceutically acceptable salt thereof, wherein G² is

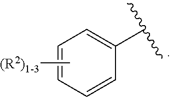

E29. The compound of any one of E22-E25, or a pharmaceutically acceptable salt thereof, wherein G² is the 5- to 12-membered heteroaryl.

E30. The compound of E29, or a pharmaceutically acceptable salt thereof, wherein the 5- to 12-membered heteroaryl at G² is furanyl, thiophenyl, pyrrolyl, or imidazolyl.

E31. The compound of E30, or a pharmaceutically acceptable salt thereof, wherein G² is

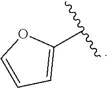 [SMILES: *c1ccco1]

E32. The compound of any one of E22-E31, or a pharmaceutically acceptable salt thereof, wherein R², at each occurrence, is independently selected from the group consisting of halogen, C₁₋₆alkyl, —OC₁₋₄alkyl, —NH₂, —OC₁₋₂fluoroalkyl, C₁₋₂fluoroalkyl, or —NO₂.

E33. The compound of any one of E22-E32, or a pharmaceutically acceptable salt thereof, wherein G³ is the 5- to 12-membered heteroaryl.

E34. The compound of E33, or a pharmaceutically acceptable salt thereof, wherein the 5- to 12-membered heteroaryl at G³ is thiophenyl, furanyl, pyrrolyl, or imidazolyl.

E35. The compound of E34, or a pharmaceutically acceptable salt thereof, wherein G³ is

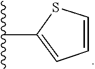 [SMILES: *c1cccs1]

E36. The compound of any one of E22-E32, or a pharmaceutically acceptable salt thereof, wherein G³ is the 6- to 12-membered aryl.

E37. The compound of E36, or a pharmaceutically acceptable salt thereof, wherein the 6- to 12-membered aryl at G³ is phenyl.

E38. The compound of E37, or a pharmaceutically acceptable salt thereof, wherein G³ is

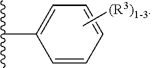

E39. The compound of any one of E22-E38, or a pharmaceutically acceptable salt thereof, wherein R³, at each occurrence, is independently selected from the group consisting of halogen, C₁₋₆alkyl, —OC₁₋₄alkyl, —NH₂, —OC₁₋₂fluoroalkyl, C₁₋₂fluoroalkyl, or —NO₂.

E40. The compound of any one of E22-E39, or a pharmaceutically acceptable salt thereof, wherein the compound of formula (II) is selected from the group consisting of:

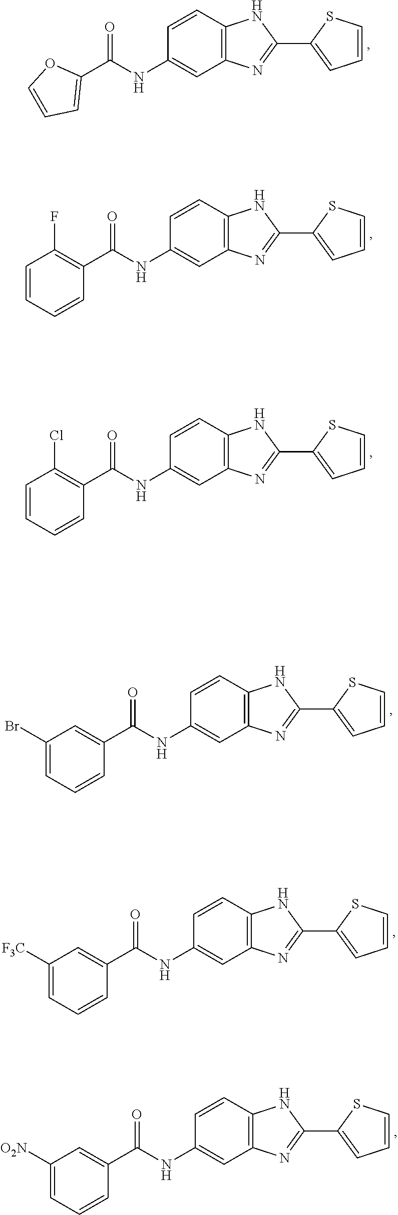 [SMILES: O=C(Nc1ccc2[nH]c(-c3cccs3)nc2c1)c1ccco1 ; O=C(Nc1ccc2[nH]c(-c3cccs3)nc2c1)c1ccccc1F ; O=C(Nc1ccc2[nH]c(-c3cccs3)nc2c1)c1ccccc1Cl ; O=C(Nc1ccc2[nH]c(-c3cccs3)nc2c1)c1cccc(Br)c1 ; O=C(Nc1ccc2[nH]c(-c3cccs3)nc2c1)c1cccc(C(F)(F)F)c1 ; O=C(Nc1ccc2[nH]c(-c3cccs3)nc2c1)c1cccc([N+](=O)[O-])c1]

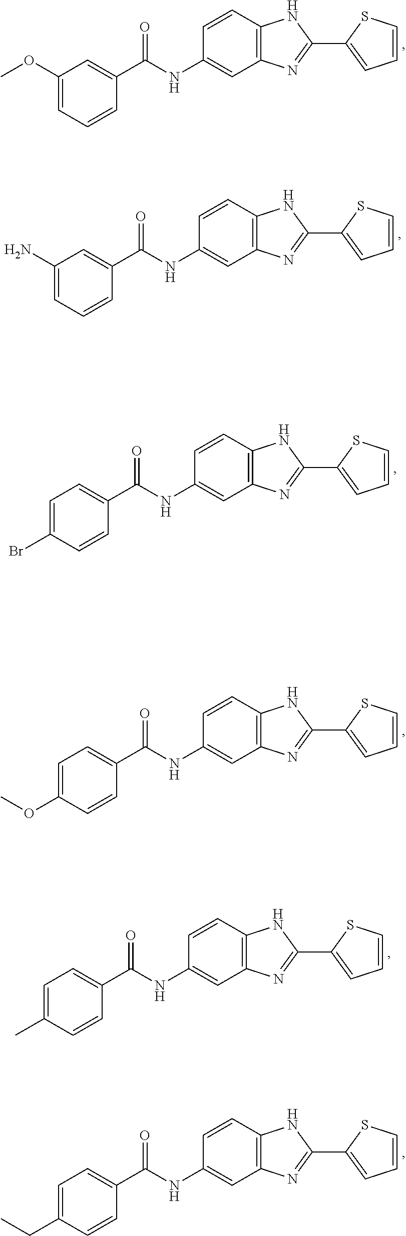 [SMILES: COc1cccc(C(=O)Nc2ccc3[nH]c(-c4cccs4)nc3c2)c1 ; Nc1cccc(C(=O)Nc2ccc3[nH]c(-c4cccs4)nc3c2)c1 ; O=C(Nc1ccc2[nH]c(-c3cccs3)nc2c1)c1ccc(Br)cc1 ; COc1ccc(C(=O)Nc2ccc3[nH]c(-c4cccs4)nc3c2)cc1 ; Cc1ccc(C(=O)Nc2ccc3[nH]c(-c4cccs4)nc3c2)cc1 ; CCc1ccc(C(=O)Nc2ccc3[nH]c(-c4cccs4)nc3c2)cc1]

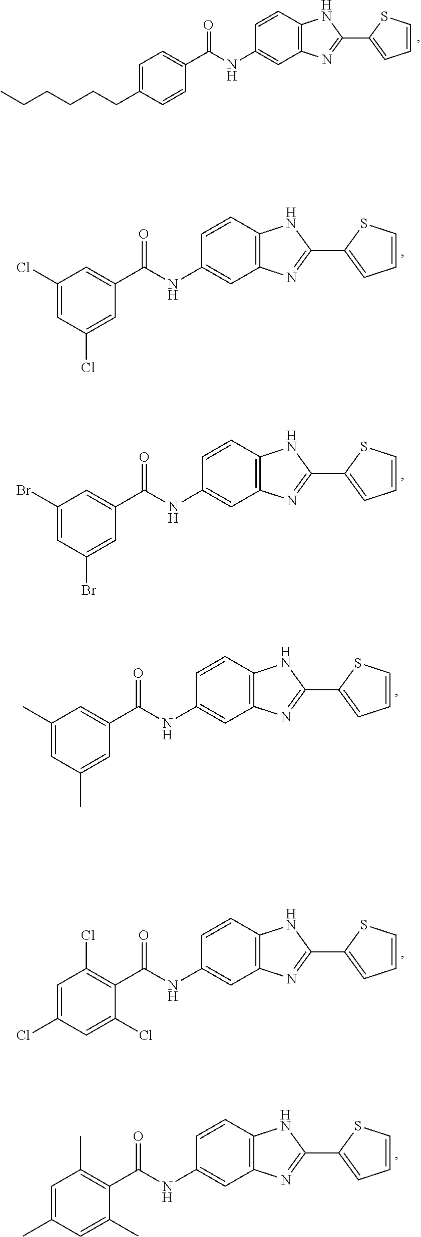 [SMILES: CCCCCCc1ccc(C(=O)Nc2ccc3[nH]c(-c4cccs4)nc3c2)cc1 ; O=C(Nc1ccc2[nH]c(-c3cccs3)nc2c1)c1cc(Cl)cc(Cl)c1 ; O=C(Nc1ccc2[nH]c(-c3cccs3)nc2c1)c1cc(Br)cc(Br)c1 ; Cc1cc(C)cc(C(=O)Nc2ccc3[nH]c(-c4cccs4)nc3c2)c1 ; O=C(Nc1ccc2[nH]c(-c3cccs3)nc2c1)c1c(Cl)cc(Cl)cc1Cl ; Cc1cc(C)c(C(=O)Nc2ccc3[nH]c(-c4cccs4)nc3c2)c(C)c1]

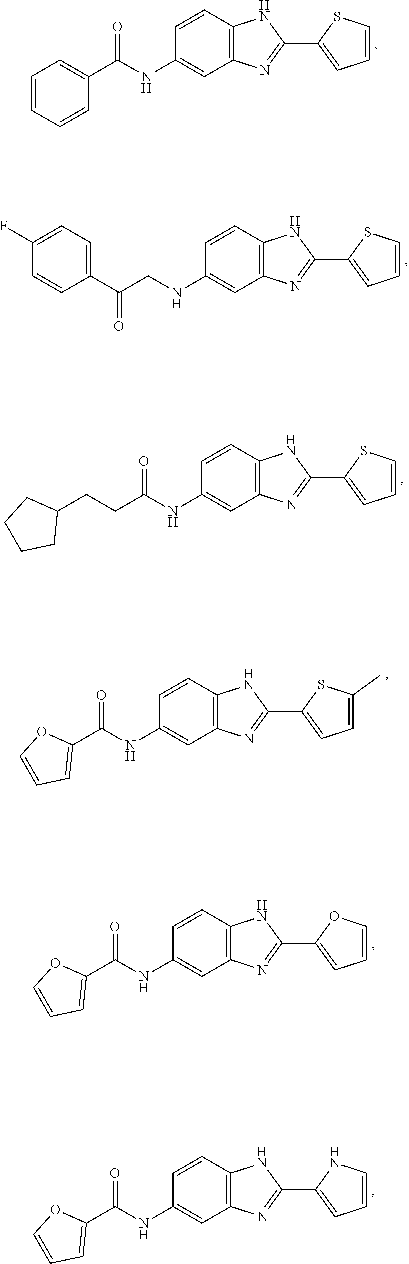 [SMILES: O=C(Nc1ccc2[nH]c(-c3cccs3)nc2c1)c1ccccc1 ; O=C(CNc1ccc2[nH]c(-c3cccs3)nc2c1)c1ccc(F)cc1 ; O=C(CCC1CCCC1)Nc1ccc2[nH]c(-c3cccs3)nc2c1 ; Cc1ccc(-c2nc3cc(NC(=O)c4ccco4)ccc3[nH]2)s1 ; O=C(Nc1ccc2[nH]c(-c3ccco3)nc2c1)c1ccco1 ; O=C(Nc1ccc2[nH]c(-c3ccc[nH]3)nc2c1)c1ccco1]

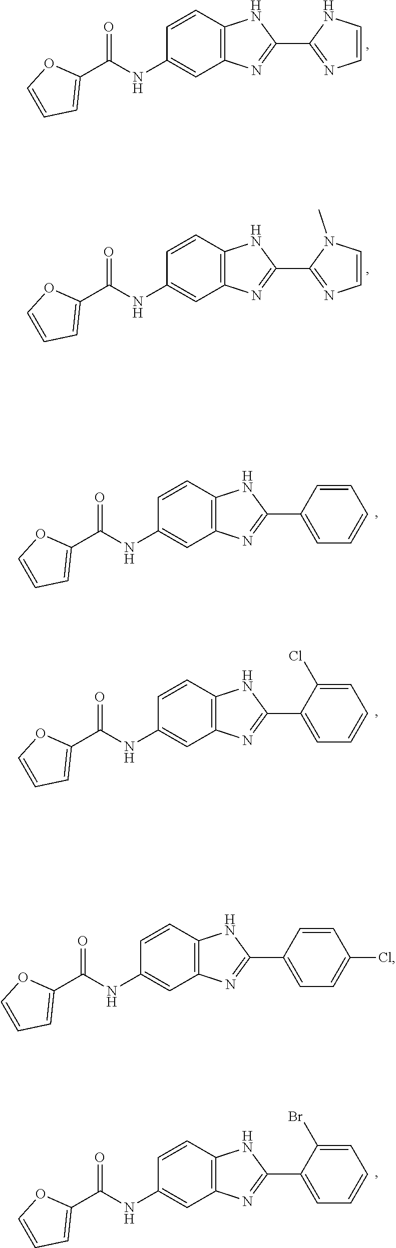 [SMILES: O=C(Nc1ccc2[nH]c(-c3ncc[nH]3)nc2c1)c1ccco1 ; Cn1ccnc1-c1nc2cc(NC(=O)c3ccco3)ccc2[nH]1 ; O=C(Nc1ccc2[nH]c(-c3ccccc3)nc2c1)c1ccco1 ; O=C(Nc1ccc2[nH]c(-c3ccccc3Cl)nc2c1)c1ccco1 ; O=C(Nc1ccc2[nH]c(-c3ccc(Cl)cc3)nc2c1)c1ccco1 ; O=C(Nc1ccc2[nH]c(-c3ccccc3Br)nc2c1)c1ccco1]

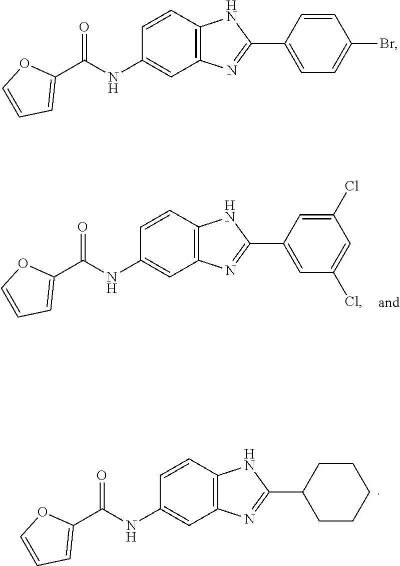 [SMILES: O=C(Nc1ccc2[nH]c(-c3ccc(Br)cc3)nc2c1)c1ccco1 ; O=C(Nc1ccc2[nH]c(-c3cc(Cl)cc(Cl)c3)nc2c1)c1ccco1 ; O=C(Nc1ccc2[nH]c(C3CCCCC3)nc2c1)c1ccco1]

### Pharmaceutical Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### General Synthesis

Compounds of formula (I) or any of its subformulas and compounds of formula (II) or any of its subformulas may be synthesized as shown in the following schemes.

Abbreviations which have been used in the Schemes that follow are:
DCM is dichloromethane;
EDC is 1-ethyl-3-(3-dimethylaminopropyl)carbodiimide;
DMAP is 4-methylaminopyridine;
Pd/C is palladium on carbon;
MeOH is methanol;
CDI is carbonyl diimidazole; and
TEA is triethylamine.

General Scheme 1, below, illustrates a general method for preparing compounds of formula (I), described herein.

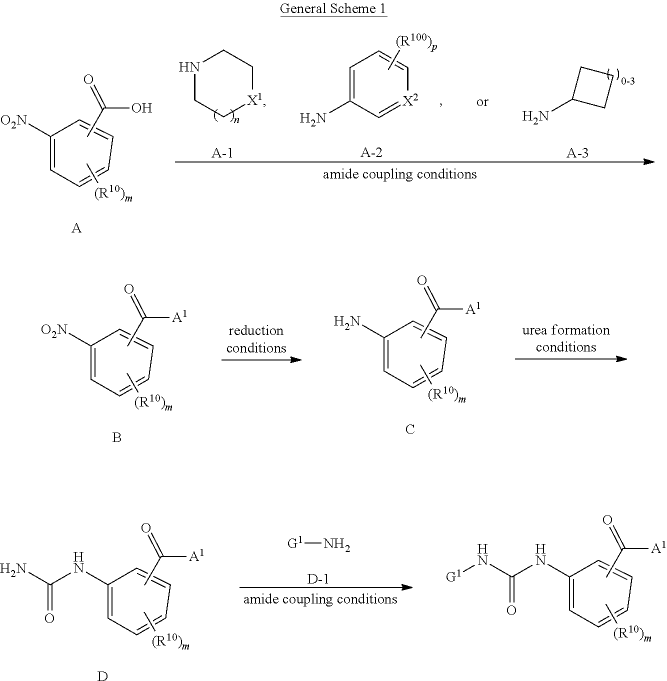

As shown in General Scheme 1 above, compounds of formula (I) may be prepared by coupling a nitrobenzoic acid of formula A with an amine of formula A-1, A-2, or A-3 under suitable amide coupling conditions (e.g., in the presence of EDC and DMAP in DCM at room temperature) to produce intermediates of formula B. Intermediates of formula B may be reduced to intermediates of formula C under suitable reduction conditions (e.g., in the presence of Pd/C and H₂ gas in MeOH). Intermediates of formula C may be reacted under suitable urea formation conditions (e.g., in the presence of isocyanate in DCM) to generate intermediate ureas of formula D. Intermediate ureas of formula D may be reacted with an amine of formula D-1 under suitable amide coupling conditions (e.g. in the presence of CDI and TEA at room temperature) to provide compounds of formula (I).

General Scheme 2, below, illustrates an example method for preparing compounds of formula (II-a), described herein.

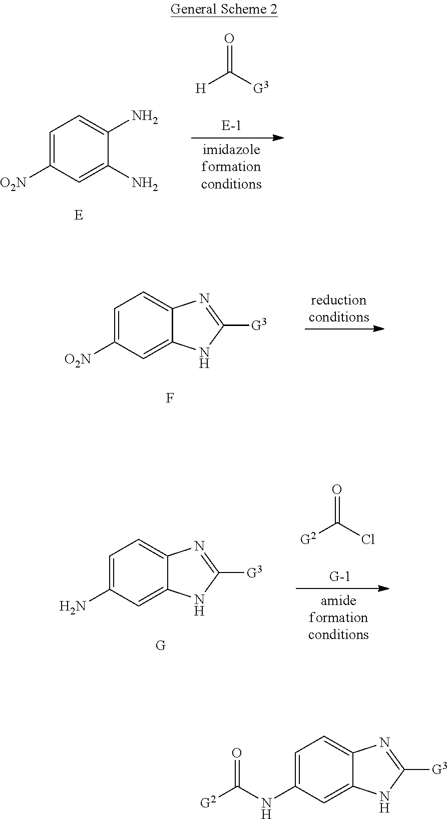

As shown in General Scheme 2 above, in some instances, various compounds of formula (II) may be prepared by reacting 4-nitrobenzene-1,2-diamine (E) with an aldehyde of formula E-1 to form nitro-benzimidazole intermediates of formula F under suitable imidazole formation conditions (e.g., in water at about 50° C.). The nitro-benzimidazole intermediates of formula F may be reduced under suitable conditions (e.g., in the presence of Pd/C and H₂ gas in MeOH) to form amino-benzimidazole intermediates of formula G. Intermediates of formula G may be reacted with an acid chloride of formula G-1 under suitable amide formation conditions (e.g., in the presence of TEA in DCM followed by an acidic work-up) to provide various compounds of formula (II).

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction may include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### Pharmaceutical Compositions

The disclosed compounds may be incorporated into pharmaceutical compositions suitable for administration to a subject (such as a patient, which may be a human or non-human animal, such as a mammal).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount may also be one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

It will be appreciated that appropriate dosages of the compounds, and compositions comprising the compounds, can vary from patient to patient. Determining the optimal dosage may involve the balancing of the level of therapeutic benefit against any risk or deleterious side effects of the treatments of the present invention. The selected dosage level may depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration may ultimately be at the discretion of the physician, although the dosage may be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the compound may be in the range of about 100 μg to about 250 mg per kilogram body weight of the subject per day.

The composition may be administered once, on a continuous basis (e.g. by an intravenous drip), or on a periodic/intermittent basis, including, but not limited to, about once per hour, about once per two hours, about once per four hours, about once per eight hours, about once per twelve hours, about once per day, about once per two days, about once per three days, about twice per week, about once per week, and about once per month. The composition may be administered until a desired reduction of symptoms is achieved.

The present compounds, compositions, and methods may be administered as part of a therapeutic regimen along with other treatments appropriate for the particular injury or disease being treated.

For example, a therapeutically effective amount of a compound of formula (I) or a compound of formula (II), may be, but is not limited to, about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Examples of materials which can serve as pharmaceutically acceptable carriers include, but are not limited to: sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as, but not limited to, propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration, among others. Techniques and formulations may be found in “Remington's Pharmaceutical Sciences”, (Meade Publishing Co., Easton, Pa.). Therapeutic compositions may be sterile and stable under the conditions of manufacture and storage.

The route by which the disclosed compounds are administered, and the form of the composition will dictate the type of carrier to be used. The composition may be in a variety of forms suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration may include at least one of diluents, lubricants, binders, disintegrants, colorants, flavors, sweeteners, antioxidants, preservatives, glidants, solvents, suspending agents, wetting agents, surfactants, combinations thereof, and others. All carriers may be optional in the compositions.

Suitable diluents may include, but are not limited to, sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition may be about 50 to about 90%.

Suitable lubricants may include, but are not limited to, silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition may be about 5 to about 10%.

Suitable binders may include, but are not limited to, polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition may be about 5 to about 50%.

Suitable disintegrants may include, but are not limited to, agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition may be about 0.1 to about 10%.

Suitable colorants may include, but are not limited to, a colorant such as an FD&C dye. When used, the amount of colorant in a systemic or topical composition may be about 0.005 to about 0.1%.

Suitable flavors may include, but are not limited to, menthol, peppermint, and fruit flavors. The amount of flavor(s), when used, in a systemic or topical composition may be about 0.1 to about 1.0%.

Suitable sweeteners include, but are not limited to, aspartame and saccharin. The amount of sweetener(s) in a systemic or topical composition may be about 0.001 to about 1%.

Suitable antioxidants may include, but are not limited to, butylated hydroxyanisole (“BHA”), butylated hydroxytoluene (“BHT”), and vitamin E. The amount of antioxidant(s) in a systemic or topical composition may be about 0.1 to about 5%.

Suitable preservatives may include, but are not limited to, benzalkonium chloride, methyl paraben and sodium benzoate. The amount of preservative(s) in a systemic or topical composition may be about 0.01 to about 5%.

Suitable glidants may include, but are not limited to, silicon dioxide. The amount of glidant(s) in a systemic or topical composition may be about 1 to about 5%.

Suitable solvents may include, but are not limited to, water, isotonic saline, ethyl oleate, glycerine, hydroxylated castor oils, alcohols such as ethanol, and phosphate buffer solutions. The amount of solvent(s) in a systemic or topical composition may be from about 0 to about 100%.

Suitable suspending agents may include, but are not limited to, AVICEL RC-591 (from FMC Corporation of Philadelphia, PA) and sodium alginate. The amount of suspending agent(s) in a systemic or topical composition may be about 1 to about 8%.

Suitable surfactants may include, but are not limited to, lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition may be about 0.1% to about 5%.

Although the amounts of components in the systemic compositions may vary depending on the type of systemic composition prepared, systemic compositions may include 0.01% to 50% of active [e.g., compound of formula (I)] and 50% to 99.99% of one or more carriers. Compositions for parenteral administration may include 0.1% to 10% of actives and 90% to 99.9% of a carrier including a diluent and a solvent.

Compositions for oral administration can have various dosage forms. For example, solid forms may include, but are not limited to, tablets, capsules, granules, and bulk powders. These oral dosage forms may include a safe and effective amount, usually at least about 5%, and more particularly from about 25% to about 50% of actives. The oral dosage compositions may include about 50% to about 95% of carriers, and more particularly, from about 50% to about 75%.

Tablets can be compressed, tablet triturates, enteric-coated, sugar-coated, film-coated, or multiple-compressed. Tablets may include an active component, and a carrier comprising ingredients selected from, but not limited to, diluents, lubricants, binders, disintegrants, colorants, flavors, sweeteners, glidants, and combinations thereof. Specific diluents may include, but are not limited to, calcium carbonate, sodium carbonate, mannitol, lactose and cellulose. Specific binders may include, but are not limited to, starch, gelatin, and sucrose. Specific disintegrants may include, but are not limited to, alginic acid and croscarmelose. Specific lubricants may include, but are not limited to, magnesium stearate, stearic acid, and talc. Specific colorants may be the FD&C dyes, which can be added for appearance. Chewable tablets may contain sweeteners including, but not limited to, aspartame and saccharin, or flavors including, but not limited to, menthol, peppermint, fruit flavors, or a combination thereof.

Capsules (including implants, time release and sustained release formulations) may include an active compound [e.g., a compound of formula (I)], and a carrier including one or more diluents disclosed above in a capsule comprising gelatin. Granules may comprise a disclosed compound, and may include glidants, such as silicon dioxide, to improve flow characteristics. Implants may be of the biodegradable or the non-biodegradable type.

The selection of ingredients in the carrier for oral compositions may depend on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings may include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms may include, but are not limited to, aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions may include a disclosed compound and a carrier, namely, a carrier selected from diluents, colorants, flavors, sweeteners, preservatives, solvents, suspending agents, and surfactants. Peroral liquid compositions may include one or more ingredients selected from colorants, flavors, and sweeteners.

For parenteral administration, the agent can be dissolved or suspended in a physiologically acceptable diluent, such as, e.g., water, buffer, oils with or without solubilizers, surface-active agents, dispersants, or emulsifiers. As oils, for example and without limitation, olive oil, peanut oil, cottonseed oil, soybean oil, castor oil and sesame oil may be used. More generally, for parenteral administration, the agent may be in the form of an aqueous, lipid, oily or other kind of solution or suspension or even may be administered in the form of liposomes or nano-suspensions.

The term “parenterally,” as used herein, refers to modes of administration which may include intravenous, intramuscular, intraperitoneal, intrasternal, subcutaneous and intraarticular injection and infusion.

Other compositions useful for attaining systemic delivery of the subject compounds may include sublingual, buccal and nasal dosage forms. Such compositions may include one or more of soluble filler substances such as diluents including, but not limited to, sucrose, sorbitol and mannitol; and binders including, but not limited to, acacia, microcrystalline cellulose, carboxymethyl cellulose, and hydroxypropyl methylcellulose. Such compositions may further include lubricants, colorants, flavors, sweeteners, antioxidants, and glidants.

The pharmaceutical compositions of the present invention may also be administered by nasal aerosol or inhalation through the use of a nebulizer, a dry powder inhaler or a metered dose inhaler. Such compositions may be prepared according to techniques well-known in the art of pharmaceutical formulation and may be prepared as solutions in saline, employing benzyl alcohol or other suitable preservatives, absorption promoters to enhance bioavailability, hydrofluorocarbons, and/or other conventional solubilizing or dispersing agents.

Aerosol propellants may be required where the pharmaceutical composition is to be delivered as an aerosol under significant pressure. Such propellants may include, e.g., acceptable fluorochlorohydrocarbons including, but not limited to, dichlorodifluoromethane, dichlorotetrafluoroethane, and trichloromonofluoromethane; nitrogen; or a volatile hydrocarbon including, but not limited to, butane, propane, isobutane, or mixtures thereof.

The disclosed compounds may be topically administered. Topical compositions that can be applied locally to the skin may be in any suitable form including, but not limited to, solids, solutions, oils, creams, ointments, gels, lotions, shampoos, leave-on and rinse-out hair conditioners, milks, cleansers, moisturizers, sprays, skin patches, and the like. Topical compositions may include: a disclosed compound (e.g., a compound of formula (I)), and a carrier. The carrier of the topical composition may aid penetration of the compounds into the skin. The carrier may further include one or more optional components.

The amount of the carrier employed in conjunction with a disclosed compound may be sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier may include a topical carrier. Suitable topical carriers may include, but are not limited to, one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof. Carriers for skin applications may include propylene glycol, dimethyl isosorbide, and water. For example, carriers for skin applications may include, but are not limited to, phosphate buffered saline, isotonic water, deionized water, monofunctional alcohols, symmetrical alcohols.

The carrier of a topical composition may further include one or more ingredients selected from emollients, propellants, solvents, humectants, thickeners, powders, fragrances, pigments, and preservatives, all of which may be optional.

Suitable emollients may include, but are not limited to, stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin may include stearyl alcohol and polydimethylsiloxane. The amount of emollient(s) in a skin-based topical composition may be about 5% to about 95%.

Suitable propellants may include, but are not limited to, propane, butane, isobutane, dimethyl ether, carbon dioxide, nitrous oxide, and combinations thereof. The amount of propellant(s) in a topical composition may be about 0% to about 95%.

Suitable solvents may include, but are not limited to, water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents may include ethyl alcohol and homotopic alcohols. The amount of solvent(s) in a topical composition may be about 0% to about 95%.

Suitable humectants may include, but are not limited to, glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition may be about 0% to about 95%.

The amount of thickener(s) in a topical composition may be about 0% to about 95%.

Suitable powders may include, but are not limited to, beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition may be about 0% to about 95%.

The amount of fragrance in a topical composition may be about 0% to about 0.5%, such as about 0.001% to about 0.1%. Suitable pH adjusting additives may include, but are not limited to, HCl or NaOH in amounts sufficient to adjust the pH of a topical pharmaceutical composition.

### Bacterial Diseases and Disorders

A “disease” is a state of health of an animal wherein the animal cannot maintain homeostasis, and wherein if the disease is not ameliorated then the animal's health continues to deteriorate. In contrast, a “disorder” in an animal is a state of health in which the animal can maintain homeostasis, but in which the animal's state of health is less favorable than it would be in the absence of the disorder. Left untreated, a disorder does not necessarily cause a further decrease in the animal's state of health. A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

A “therapeutic” treatment is a treatment administered to a subject who exhibits signs or symptoms of pathology disease or disorder, for the purpose of diminishing or eliminating those signs or symptoms. As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder. The term “treating” or “treatment” also refers to a reduction in the severity of one or more symptoms by about 10%, about 20%, about 30%, about 40%, about 50%, about 60%, about 70%, about 80%, about 90% or about 100%.

As used herein, the term “microbial colonization” refers to the formation of compact population groups of the same type of microorganism (such as bacteria), such as the colonies that develop when a microbial (such as bacterial) cell begins reproducing. The microbial colonization (such as bacterial colonization) may or may not cause disease symptoms. Decolonization refers to a reduction in the number of microbial (such as bacterial) organisms. When the microbial organisms are completely decolonized, the microbial organisms have been eradicated and are non-detectable.

As used herein, the term “bacteria” includes, but is not limited to, references to organisms of the following classes and strains thereof:
1) Gram-positive cocci, such as Staphylococci (e.g. Staph. aureus, Staph. epidermidis, Staph. saprophyticus, Staph. auricularis, Staph. capitiscapitis, Staph. cureolyticus, Staph. caprae, Staph. cohnii, Staph. c. urealyticus, Staph. equorum, Staph. gallinarum, Staph. haemolyticus, Staph. hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus);
2) Streptococci (e.g. beta-haemolytic, pyogenic streptococci (such as Strept. agalactiae, Strept. canis, Strept. dysgalactiae, Strept. dysgalactiae equisimilis, Strept. equi, Strept. equi zooepidemicus, Strept. iniae, Strept. porcinus and Strept. pyogenes), microaerophilic, pyogenic streptococci (Streptococcus “milleri”, such as Strept. anginosus, Strept. constellatus, Strept. constellatus pharyngidis and Strept. intermedius), oral streptococci of the “mitis” (alpha-haemolytic—Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups, Strept. acidominimus, Strept. bovis, Strept. faecalis, Strept. equinus, Strept. pneumoniae and Strept. suis, or Streptococci alternatively classified as Group A, B, C, D, E, G, L, P, U or V Streptococcus);
3) Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri;
4) Bacillaceae, such as Bacillus anthracis, Bacillus subtilis, Bacillus thuringiensis, Bacillus stearothermophilus and Bacillus cereus;
5) Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella morganii, Salmonella (Salmonella enterica and Salmonella typhi), Shigella (e.g. Shigella dysenteriae, Shigella flexneri, Shigella boydli and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis);
6) Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius);
7) Helicobacter (e.g. Helicobacter pylori, Helicobacter cinaedi and Helicobacter fennelliae);
8) Acinetobacter (e.g. A. baumannii, A. calcoaceticus, A. haemolyticus, A. johnsonii, A. junii, A. iwoffi and A. radioresistens);
9) Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri);
10) Bacteroides fragilis;
11) Peptococcus (e.g., Peptococcus niger);
12) Peptostreptococcus
13) Clostridium (e.g. C. perfringens, C. difficile, C. botulinum, C. tetani, C. absonum, C. argentinense, C. baratii, C. bifermentans, C. bejerinckii, C. butyricum, C. cadaveris, C. camis, C. celatum, C. clostridioforme, C. cochlearium, C. cocleatum, C. fallax, C. ghonii, C. glycolicum, C. haenolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium);
14) Mycoplasma (e.g., M. pneumoniae, M hominis, M. genitalium and M. urealyticum);
15) Mycobacteria (e.g., Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Haemophilus (e.g. Haemophilus influenzae, Haemophilus ducreyi, Haemophilus aegyptius, Haemophilus parainfluenzae, Haemophilus haemolyticus and Haemophilus parahaemolyticus);
16) Actinobacillus (e.g., Actinobacillus actinomycetemcomitans, Actinobacillus equuli);
17) Actinobacillus hominis, Actinobacillus lignieresii, Actinobacillus suis and Actinobacillus ureae;
18) Actinomyces (e.g. Actinomyces israelii);
19) Brucella (e.g. Brucella abortus, Brucella canis, Brucella melintensis and Brucella suis);
20) Campylobacter (e.g. Campylobacter jejuni, Campylobacter coli, Campylobacter lari and Campylobacter fetus);
21) Listeria monocytogenes;
22) Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus);
23) Erysipelothrix rhusopathiae;
24) Corynebacteriaceae (e.g. Corynebacterium diphtheriae, Corynebacterium jeikeum and Corynebacterium urealyticum);
25) Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum); Pasteurella (e.g. Pasteurella aerogenes, Pasteurella bettyae, Pasteurella canis, Pasteurella dagmatis, Pasteurella gallinarum, Pasteurella haemolytica, Pasteurella multocida, Pasteurella multocida gallicida, Pasteurella multocida septica, Pasteurella pneumotropica and Pasteurella stomatis);
26) Bordetella (e.g. Bordetella bronchiseptica, Bordetella hinzii, Bordetella holmseii, Bordetella parapertussis, Bordetella pertussis and Bordetella trematum);
27) Nocardiaceae, such as Nocardia (e.g. Nocardia asteroides and Nocardia brasiliensis);
28) Rickettsia (e.g. Ricksettsii or Coxiella burnetii),
29) Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachemii, Legionalla micdadei, Legionalla oakridgensis, Legionalla pneumophila, Legionalla sainthelensi, Legionalla tucsonensis;
30) Legionalla wadsworthii;
31) Moraxella catarrhalis;
32) Cyclospora cayetanensis;
33) Entamoeba histolytica;
34) Giardia lamblia;
35) Trichomonas vaginalis;
36) Toxoplasma gondii;
37) Stenotrophomonas maltophilia;
38) Burkholderia cepacia; Burkholderia mallei and Burkholderia pseudomallei;
39) Francisella tularensis;
40) Gardnerella (e.g. Gardneralla vaginalis and Gardneralla mobiluncus); Streptobacillus moniliformis;
41) Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena);
42) Bartonella (Bartonella bacilliformis, Bartonella clarridgeiae, Bartonella elizabethae, Bartonella henselae, Bartonella quintana and Bartonella vinsonii arupensis);
43) Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii);
44) Spirillium (e.g. Spirillum minus);
45) Baceteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus);
46) Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans); Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae);
47) Fusobacterium (e.g. F. gonadiaformans, F. mortiferum, F. naviforme, F. necrogenes, F. necrophorum, F. necrophorum fundiliforme, F. nucleatum, F. nucleatum fusiforme, F. nucleatum polymorphum, F. nucleatum vincentii, F. periodonticum, F. russii, F. ulcerans and F. varium);
48) Chlamydia (e.g. Chlamydia trachomatis);
49) Cryptosporidium (e.g. C. parvum, C. hominis, C. canis, C. felis, C. meleagridis and C. muris);
50) Chlamydophila (e.g. Chlamydophila abortus (Chlamydia psittaci), Chlamydophila pneumoniae (Chlamydia pneumoniae) and Chlamydophila psittaci (Chlamydia psittaci));
51) Leuconostoc (e.g. Leuconostoc citreum, Leuconostoc cremoris, Leuconostoc dextranicum, Leuconostoc lactis, Leuconostoc mesenteroides and Leuconostoc pseudomesenteroides);
52) Gemella (e.g. Gemella bergeri, Gemella haemolysans, Gemella morbillorum and Gemella sanguinis); and
53) Ureaplasma (e.g. Ureaplasma parvum and Ureaplasma urealyticum).

### Penicillin Binding Protein 4

Penicillin Binding Proteins (PBPs) are enzymes that participate in the synthesis of peptidoglycan. Specifically, PBPs catalyze peptidoglycan glycan peptide side chain crosslinking, thereby conferring cell wall rigidity. There are four sub-types of PBPs and these subtypes include PBP1, PBP2, PBP3, and PBP4. β-lactams are a class of antibiotics that act as substrate analogs and covalently bind to the active- and inactive-site of PBPs. Antibiotic resistant bacterial strains have developed defense against antibiotics by evolving new proteins that do not interact with antibiotics. This defense mechanism has facilitated the emergence of isoforms of the PBP sub-types. One isoform that has evolved is the PBP2a isoform. PBP2a is a transpeptidase that, in comparison to PBP2, displays a structural rearrangement that narrows the protein's substrate binding cleft, which in turn lowers its R-lactam affinity and allows the enzyme to maintain transpeptidase function in the presence of β-lactams.

Studies performed in S. aureus bacterial strains engineered to express the PBP2a protein no longer have identified an alternative mechanism for β-lactam resistance. These studies identified PBP4 as a contributor to resistance to β-lactam resistance. In a separate series of genetic and chemical genomic studies, PBP4, as opposed to PBP2a, was identified as a key factor in methicillin resistant Staphylococcus aureus phenotype of community acquired methicillin-resistant strains. Emerging studies have revealed that in addition to modulating S. aureus 1-lactam resistance, PBP4 may act as a virulence factor that contributes to the organism's propensity to cause reoccurring osteomyelitis. More directly, transmission electron microscopy of animal- and clinical cases—of osteomyelitis revealed that S. aureus has the ability to invade cortical bone osteocyte lancuno-canalicular networks (OLCN). More directly, transmission electron microscopy of animal- and clinical cases—of osteomyelitis revealed that S. aureus has the ability to invade cortical bone osteocyte lancuno-canalicular networks (OLCN). In a genetic screen using Microfluidic-Silicon Membrane-Canalicular Arrays (μSiM-CA) with engineered 500 nm size pores designed to mimic the OLCN orifice, it has been observed that S. aureus PBP4 is required for traversing the membrane pores, thus indicating the protein is required for OLCN bacterial colonization. Generation of a PBP4 variant lacking activity eliminates the ability of a bacterial to invade and colonize OLCN in animals. Thus, putative PBP4 inhibitors may reduce S. aureus OLCN invasion.

### EXAMPLES

### General Materials and Methods

Bacterial Strains and Chemicals. The bacterial strains used in these studies include: USA300, USA300Δpbp4, USA300Δpbp4 pPBP4, COL, COLn, COLnex, CRB, and CRBΔpbp4. S. aureus strain USA300 is a predominant cause of U.S. community-associated MRSA infections, whereas strain COL is a commonly studied hospital-associated MRSA strain. The S. aureus USA300 pbp4-null strain (USA300Δpbp4) and PBP4 complementation strain (USA300Δpbp4 pPBP4) have been previously described. Strain COLn is a laboratory derived tetracycline susceptible derivative of COL, whereas COLnex is a methicillin susceptible COLn derivative that has been cured of SCCmec thereby removing the mecA, which encodes PBP2a, and has been previously described. Strain CRB is a well-studied MRSA COLnex derivative that contains two amino acid substitutions (E183A and F241R) near the PBP4 active site and a 36-bp duplication 290-bp upstream of the pbp4 open reading frame leading to 20-40-fold overexpression of the gene. All strains were grown in Mueller Hinton (MH) broth at 37° C., as indicated. Penicillin, streptomycin, ciprofloxacin, mupirocin, meropenem, and mitomycin C were purchased from Fisher Scientific® (Waltham, MA), whereas ceftobiprole was obtained from MedChemExpress® LLC (Monmouth Junction, NJ). The 30,000-member DIVERSET®-EXP small molecule chemical library (Blocks 3-5) used in these studies was purchased from ChemBridge® Corporation (San Diego, CA).

High Throughput Screen for PBP4 Inhibitors. A 30,000-member ChemBridge® small molecule compound library was screened for agents that potentiated the antimicrobial performance of ciprofloxacin toward S. aureus strain USA300, effectively phenocopying a USA300Δpbp4 strain. As a prerequisite for screening, Z-factor analyses were performed to identify the optimal ciprofloxacin concentration that reproducibly allowed distinction between the growth of USA300 and USA300Δpbp4 strains. To do so, in 96-well format strains, USA300 and the pbp4 deletion strain were used to inoculate (˜1×10⁴ colony forming units; CFUs) wells of alternative rows of a microtiter plate containing 100 μL MH media (final volume) supplemented with either 0, 1, 2, or 4 μg mL⁻¹ ciprofloxacin. Plates were incubated overnight, bacterial growth within individual wells was measured by OD_{600 nm}, and Z-factor was calculated; results revealed that MH media supplemented with 2 μg mL-1 ciprofloxacin reproducibly distinguished between wild type and pbp4 mutant cells (Z-factor of 0.314). To screen for compounds that phenocopied the ciprofloxacin susceptibility of pbp4 mutant cells, a total of 1×10⁴ CFUs of strain USA300 (10 μL) were inoculated into individual wells of a 96-well microtiter plate containing 88 μL of MH media supplemented with 2 μg mL⁻¹ ciprofloxacin, and 2 μL of test compound was added (50 μM final concentration). Plates were incubated at 37° C. for 16 hours and growth was measured by the naked eye. Compounds that prevented growth were considered hits. To counter select for compounds with inherent antimicrobial activity, hits were directly assessed for stand-alone antimicrobial activity toward S. aureus USA300 by repeat testing (50 μM) in the absence of ciprofloxacin.

Eukaryotic Toxicity Testing. Two rounds of eukaryotic toxicity testing were performed. Initially hits of interest were evaluated for antifungal activity in high throughput manner. To do so, 10⁵ CFU of Saccharomyces cerevisiae YSB1001 cells (10 μL) were inoculated into individual wells of a microtiter plate containing yeast peptone dextrose media (88 μL) and 50 μM of each hit (2 μL). Plates were incubated at 37° C. for 16 hours and growth was visually measured by the naked eye; any compound which appeared to prevent growth was considered to exhibit eukaryotic cytotoxicity and triaged. More extensive mammalian cytotoxicity testing was performed on the indicated highest priority hits of interest following International Organization for Standardization guidelines. Briefly, human liver epithelial cells (HEPG2) were cultured in Dulbecco's modified Eagle medium (DMEM; Fisher Scientific®) supplemented with 10% heat inactivated fetal bovine serum (FBS; Corning Life Sciences, Corning, NY) and 1% penicillin and streptomycin. Cells were incubated at 37° C. with 5% CO₂ in Nunc™ tissue culture flasks (Roskilde, Denmark) until reaching 70% confluency. Cells were removed with 0.25% trypsin (Fisher Scientific®), resuspended in fresh medium, and used to seed approximately 2.5×10⁵ mL⁻¹ cells into individual wells of a 96 well tissue culture microplate (Nunc™) containing 200 μL of fresh medium, and incubated for 24 hours. Media was then removed, and cells were washed with 1× phosphate buffered saline (PBS) and fresh media supplemented with 5% per volume of a compound of interest at final concentrations ranging from 0 μM to 400 μM were added; DMSO and 125 μg·mL⁻¹ mitomycin C served as negative and positive toxicity controls. Mixtures were incubated for 20-24 h at 37° C. with 5% CO₂, the media was removed and replaced with 100 μL fresh media and 10 μL of 12 mM (3-(4,5-dimethylthiazol-2-yl)-2,5-diphenyltetrazolium bromide (MTT) reagent (Cyquant™ Cell Viability kit; Invitrogen™, Carlsbad, CA) was added to each well. Cells were incubated at 37° C. with 5% CO₂ for 2-4 hour, 85 μL of media was removed and the remainder was mixed with 50 μL DMSO and incubated for an additional 10 minutes at 37° C. in the dark. Using a SpectraMax® M5 microplate reader, cell viability was recorded as absorbance at OD_{540 nm}. All compounds were tested in triplicate and cell viability was expressed as a percent viability of treated cells in comparison to mock treated cells.

Fractional Inhibitory Concentration Index (FICI). Fractional inhibitory testing was performed in checkerboard format to determine whether putative PBP4 inhibitors potentiated the antimicrobial effects of β-lactams toward S. aureus strains USA300 and CRB, as previously described (Chojacnki, et al. Journal of Antimicrobial Chemotherapy, 2003, 52, 1). To do so, in triplicate 10⁵ CFU of each strain (10 μL) was added to individual wells of a microtiter plate containing 90 μL MH media. Each row of each plate was supplemented with increasing concentrations of the test PBP4 inhibitor (0, 3.125, 6.25, 12.5, 25, 50, 100, 200 and 400 μM), whereas each column was treated with increasing concentrations (0, 0.125, 0.25, 0.5, 1.0, 2, 4, 8, 16, 32, 64 and 128 μg/mL) of antibiotic (ceftobiprole, meropenem, cefepime or oxacillin). Plates were incubated at 37° C. for 16-20 hour and the lowest concentration of each test agent that inhibited bacterial growth alone or in combination was determined by the naked eye. The fractional inhibitory concentration (FIC) index was calculated using the formula: FIC of A (MIC of drug A in combination/MIC of drug A alone)+FIC B (FIC of drug B in combination/MIC of drug B alone). A synergistic interaction was defined as a FICI value of less than 0.5, an additive interaction was defined as a FICI value between 0.5 and 1.0, an indifferent interaction was defined as a FICI value between 1.0 and 4.0, and an antagonistic interaction was defined as a FICI value of more than 4.0.

S. aureus μSiM-CA Transmigration. The effect of PBP4 inhibitors on S. aureus migration through 500 nm nanoporous membranes modeling the orifices of osteocyte lacuno-canalicular networks was investigated using a μSiM-CA device, as previously described (Masters, et al. PLoS Pathogen, 2020, 16, e1008988). Briefly, SIM-CA systems were fabricated by SiMPore® Inc. (West Henrietta, NY, USA) to include a top and a bottom chamber that were separated by 400 nm thick silicon nitride membrane containing an array of 500 nm pores, allowing for quantification of bacteria that migrate from the apical (top) well to the basal (bottom) well. To measure bacterial migration through the device membrane, S. aureus strains USA300 or USA300Δpbp4 were grown overnight at 37° C. with aeration, diluted (1:100) in fresh MH media, and then grown to mid-exponential phase (OD_{600 nm}=0.2) in the presence or absence of putative PBP4 inhibitor for approximately 2 hour at 37° C. shaking with aeration. A total of 100 μL of cell mixture (˜1×10⁷ CFU) was transferred into the top reservoir of the device and incubated for 6 hour at 37° C. A total of 10 μL of media from the apical and basal reservoirs was collected, serial diluted in 0.8% NaCl, and plated on MH agar plates for bacterial enumeration.

Quantitative Reverse Transcriptase PCR. PBP4 transcript titers of bacterial cells exposed to putative PBP4 inhibitors were measured using real time quantitative PCR (RT-qPCR), as previously described (Chojnacki, et al. Antibiotics, 2021, 10, 369). Briefly, overnight cultures of S. aureus strains USA300, USA300Δpbp4, CRB, and CRBΔpbp4 were grown overnight in MH media, diluted (1:100 dilution) in fresh media and sub-cultured to a final OD_{600 nm} of 0.2 at 37° C. with aeration. Cells were treated with 0 or 25 μM of the indicated compound for 3 hour at 37° C., which did not impact bacterial growth at these conditions. Cells were washed with PBS buffer, and collected via centrifugation at 1,250×g for 10 minutes. Total bacterial RNA was isolated from cell pellets using Qiagen® RNeasy kits, following the manufacturer's guidelines for prokaryotic RNA isolation (Qiagen®, Germantown, MD). A total of 2 μg RNA was DNase treated and repurified using RNeasy kits.

To measure pbp4 expression, a total of 400 ng of total bacterial RNA was used as a template for qRT-PCR using pbp4 primers (forward, 5′-GGAATCCAGCGTCTATGACTAAA-3′; reverse, 5′-GTCTCCTGCACCCATGATAAC-3′) and a total of 4 ng of total bacterial RNA was used as a template for primers specific for 16S rRNA (forward, 5′-ACGGTCTTGCTGTCACTTATAG-3′; reverse, 5′-CACTGGTGTTCCTCCATATCTC-3′). Bacterial RNA was amplified and measured using PerfeCTa® SYBR Green FastMix® and qScript® cDNA SuperMix® kits following the manufacturer's recommendations (QuantaBio®, Beverly, MA). All samples were conducted in triplicate and normalized to 16S rRNA, averaged, and compared to untreated exponential phase wild type; RNA isolated from CRBΔpbp4 was used as a negative control.

Triton X-100 Susceptibility Studies. The effect of putative PBP4 inhibitors on bacterial growth in media supplemented with Triton X-100 was measured, as previously described (Ingravale, et al. Molecular Microbiology, 2003, 48, 1451-1466). Briefly, an overnight culture of S. aureus strains USA300 was diluted to an OD_{600 nm} of 0.03 in either fresh MH media, MH supplemented with 0.1% triton X-100 or MH supplemented with 12.5 μM of putative PBP4 inhibitor; DMSO treated cells served as a negative control, whereas strain USA300Δpbp4 served as a positive control for triton susceptibility. Cell suspensions were incubated at 37° C. with aeration and cell density (OD_{600 nm}) was recorded hourly for a total of 7-8 hours.

PBP4 BOCILLIN™ FL Binding Competition Assays. The ability of putative PBP4 inhibitors to impact the active site of recombinant PBP4 was assessed using two previously established BOCILLIN™-FL Penicillin (Penicillin V conjugated to BODIPY fluorescent dye) binding assays. First, fluorescence polarization assays were performed as previously described. Briefly, the indicated amount of the putative PBP4 inhibitor or control antibiotic was incubated with 1 μM recombinant B. subtilis PBP4 or S. aureus PBP2a in potassium phosphate buffer (40 mM K₂HPO₄ and 10 mM KH₂PO₄) for 1 hour in black 96-well microtiter plates (Corning Life Sciences®, Corning, NY). Following incubation, 1 μM (final concentration) BOCILLIN™ FL penicillin was added, and the mixture was incubated for 30 minutes. BOCILLIN™ FL fluorescence polarization was measured at (495ₙₘ excitation and 545ₙₘ emission) in a SpectraMax® M5 multimode plate reader (Molecular Devices®, San Jose, CA). All assays were repeated at least 8 times, averaged, and values falling outside of 2 standard deviations were considered outliers. Resulting data was plotted as percentage BOCILLIN™ FL binding inhibition=(T/U)×100; T=FL polarization of ((PBP4+BOCILLIN™ FL)−(PBP4+BOCILLIN™ FL+inhibitor compound)), U=FL polarization of ((PBP4+BOCILLIN™ FL)−(BOCILLIN™ FL)). Second, gel electrophoresis and fluorescence detection were used to measure BOCILLIN™ FL labeling of PBP4 treated with the known PBP4 inhibitor cefoxitin (positive control), kanamycin (negative control) or 9314848 (test compound). Briefly, 1 μM of recombinant PBP4 was mixed with 0, 50, 500, 2000, or 5000 μM 9314848 or control antibiotic in 50 mM phosphate buffer (pH 7.4) and incubated at 37° C. for 30 minutes. Next 40 μM of BOCILLIN™ FL was added to the reaction mixture and incubated for an additional 30 minutes at 37° C. Reactions were stopped by adding 5×SDS-PAGE gel loading dye, heated at 100° C. for 3 minutes and separated by electrophoresis in 12% SDS-PAGE (2 h, 120 Volts). BOCILLIN™ FL labeled PBP4 was imaged at λ=365 nm using a BioRad© Gel Doc EZ™ Imaging system and the gel was then subsequently stained with Bio-safe Coomassie Brilliant Blue stain (BioRad©, Hercules, CA, USA) to measure the amount of protein present in each reaction condition and analyzed using NIH-ImageJ software. The percent BOCILLIN™ FL binding was calculated by first normalizing the protein present in each reaction condition to that of untreated control and subsequently applying each reaction's protein normalization factor to each reaction's fluorescent signal. The percentage BOCILLIN™ FL binding inhibition was then calculated as the normalized band intensity of antibiotic/compound treated sample as compared to control sample (PBP4+BOCILLIN™ FL in the absence of compound).

Mammalian Toxicity of PBP4 Inhibitors. Cytotoxicity testing was conducted according to the guidelines of the International Organization for Standardization (ISO) 10993-5: 2009. Human liver epithelial cells (HEP G2) were cultured in Dulbecco's modified Eagle medium (DMEM; Fisher Scientific®) supplemented with 10% heat inactivated fetal bovine serum (FBS; Corning Life Sciences, Corning, NY) and 1% penicillin/streptomycin (Fisher Scientific®, Hampton, NH). Cells were incubated at 37° C. with 5% CO₂ in Nunc™ tissue culture flasks (Roskilde, Denmark). Cells were grown in monolayers until exceeding 70% confluency, removed with 0.25% trypsin (Fisher Scientific©), resuspended in fresh medium, and used to seed approximately 2.5×10⁵ cells/mL into each well of a 96 well tissue culture microplate (Nunc™) containing 200 μL of fresh medium. After 24 h of incubation, the cell medium was removed, and adherent cells were washed with 1× phosphate buffered saline (PBS). Cell media was supplemented with 5% per volume of compound at final concentrations between 50 μM to 400 μM and incubated 20-24 h at 37° C. with 5% CO₂. After incubation, the media was removed and replaced with 100 μL fresh media and 10 μL of 12 mM (3-(4,5-dimethylthiazol-2-yl)-2,5-diphenyltetrazolium bromide (MTT) reagent (Cyquant™ MTT kit; Invitrogen™, Carlsbad, CA) was added to each well. Cells were incubated at 37° C. with 5% CO₂ for 2-4 h before all but 25 μL and adding double the volume (50 μL) of DMSO, mixing, and incubating for an additional 10 minutes at 37° C., in the dark. Using a SpectraMax© M5 microplate reader, absorbance was read at 540 nm. Cells were treated with 125 μg/mL mitomycin C (Fisher Scientific®) to serve as a positive control and 1% DMSO as a negative control. Following ISO guidelines, compounds that resulted in cell viability below 70% were considered cytotoxic. All compounds were tested in triplicate and cell viability was expressed as a percent of the negative control.

Broth Microdilution Method for Minimum Inhibitory Concentration Determination with MRSA Strains. Bacteria were cultured for six hours in CAMHB and sub-cultured to 5×105 CFU/mL in CAMHB. Subcultures were aliquoted (1 mL) into culture tubes and then inoculated with compound from 100 mM stock in DMSO to a concentration of 200 PM. Samples were then aliquoted (200 μL) into the first row of wells of a 96-well microtiter plate in which wells 2-11 were prefilled with 100 μL of subculture. From row 1, 100 μL were withdrawn and transferred to row two, mixed 5-8 times. This procedure was used to serially dilute the rest of the rows through 10. Row 12 was filled with 100 μL of CAMHB. The microtiter plate was then covered in Press 'n Seal and incubated at 37° C. After 16 hours, the OD600 was measured, and MIC calculated as 90% inhibition of bacterial growth compared to the untreated control.

Broth Microdilution Method for Antibiotic Potentiation with MRSA Strains. Bacteria were cultured for 6 hours in CAMHB and sub-cultured to 5×105 CFU/mL in CAMHB. Subcultures were aliquoted (4 mL) into culture tubes and then inoculated with compound from 10 mM or 100 mM stock in DMSO to the desired concentration. Compounds were tested at 60% their MIC or at 60 μM if the MIC was above 200 μM. To a secondary culture tube, 1 mL was then aliquoted and dosed with antibiotic at the highest concentration to be tested. Bacteria treated with antibiotic alone served as the control. Samples from the secondary culture tubes were then aliquoted (200 μL) into the first row of wells of a 96-well microtiter plate. Rows 2-11 were filled (100 μL) with the remaining 4 mL of bacterial subculture containing the adjuvant. Row 12 was filled (100 μL) with uninoculated media. From row 1, 100 μL was transferred to row two and mixed 5-8 times before being transferred to row 3. This procedure was used to serial dilute the rest of the rows through 10. The microtiter plate was then covered in Press 'n Seal and incubated at 37° C. After 16 hours, the OD600 was measured, and MIC calculated.

Time-Kill Curves. Strains were cultured overnight in CAMHB and subcultured to 5×105 CFU/mL. The subculture was then transferred to culture tubes in 3 mL aliquots, which were dosed with adjuvant, oxacillin, or adjuvant plus oxacillin. One aliquot was not dosed to serve as a control. All samples were then incubated at 37° C. with shaking. At 2, 4, 6, 8, and 24-hour time points, 100 μL was taken from each sample and ten-fold diluted in CAMHB up 4-7 times. 100 μL of diluted culture was plated on Tryptic soy agar (TSA) and incubated at 37° C. overnight. The total number of bacterial colonies on each plate was determined using a SphereFlash™ colony counter (NEUTEC Group Inc.).

### Example 1: Identification of Small Molecule S. aureus PBP4 Inhibitors by High Throughput Screening (HTS)

### Development of Screening Paradigm Using Existing Antibiotics

Prior to identifying new PBP4 inhibitors, a screening paradigm to identify existing molecules that reverse the antibiotic resistance profile of wild type cells to that of a pbp4 mutant was developed. To define screening conditions three strains were employed: 1) a wild type USA300 MRSA strain, 2) an isogenic pbp4 deletion strain, and 3) a complemented strain harboring a plasmid wildtype pbp4 copy. The antibiotic susceptibility profile of each strain was measured to determine the magnitude with which PBP4 affects resistance to various antibiotics (Table 1). As expected, minimum inhibitory concentration (MIC) testing revealed that wild type USA300 displayed resistance to the β-lactam antibiotics nafcillin (8.0 μg/mL) and meropenem (4.0 μg/mL) and that resistance was reduced 2-fold for an isogenic pbp4-mutant strain, supporting earlier studies indicating that PBP4 impacts MRSA phenotype. While PBP4 seemingly did not affect tobramycin, minocycline, mupirocin, or colistin resistance, it was found that resistance to the fluoroquinolone antibiotics, ciprofloxacin and to a lesser extent levofloxacin, is likely to be impacted by PBP4. More specifically, wild type USA300 cells displayed a ciprofloxacin MIC of 4.0 μg/mL, whereas the MIC of the pbp4 mutant strain was reproducibly determined to be 1.0 μg/ml and complementation with a wild type copy of the pbp4 gene partially restored ciprofloxacin resistance (2.0 μg/mL) during these assay conditions. Taken together, these results indicated that PBP4 provides a 2-fold increase in resistance to nafcillin and meropenem, but a greater ˜4-fold resistance against the antimicrobial effects of ciprofloxacin for the USA300 strain used here. The greater dynamic range of the latter may provide a means to reliably screen for agents that inhibit PBP4 function. Additionally, the compounds that have no impact on PBP4 activity may not affect USA300 growth in the presence of ciprofloxacin, whereas compounds that inhibit PBP4 function may display a loss of growth phenotype in the presence of the antibiotic, phenocopying the pbp4-mutant.

TABLE 1
Minimum inhibitory concentration (μg/mL) of various antibiotics tested
in different methicillin-resistant Staphylococcus aureus (MRSA) strains

| Strain | NAF | MER | BPR | CIP | LEV | TOB | MIN | MUP | COL |
|---|---|---|---|---|---|---|---|---|---|
| USA300 | 8 | 4 | 0.5 | 4 | 1 | 1 | 0.5 | 0.125 | 128 |
| USA300Δpbp4 | 4 | 2 | 0.25 | 1 | 0.5 | 1 | 0.5 | 0.125 | 128 |
| USA300Δpbp4 pPBP4 | 8 | 4 | 0.5 | 2 | 1 | 1 | 0.5 | 0.125 | 128 |
| COLnex | 0.25 | 1 | 0.25 | 0.25 | 0.25 | 0.06 | 0.5 | 0.03 | 128 |
| CRB | >128 | 64 | >128 | 0.5 | 0.5 | 0.06 | 0.5 | 0.03 | 128 |
| CRBΔpbp4 | 0.25 | 1 | 0.25 | 0.25 | 0.25 | 0.06 | 0.5 | 0.03 | 128 |

NAF (nafcillin);
BPR (ceftobiprole);
MER (meropenem);
CIP (ciprofloxacin);
LEV (levofloxacin);
TOB (tobramycin);
MIN (minocycline);
MUP (mupirocin);
COL (colistin)

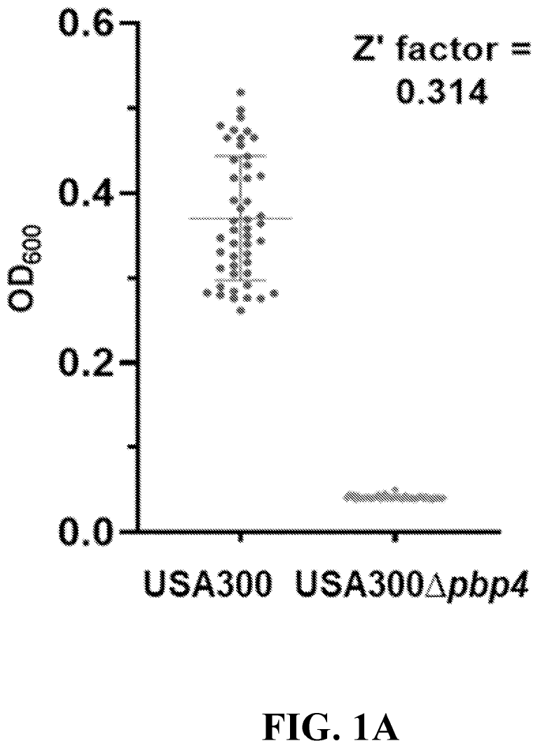

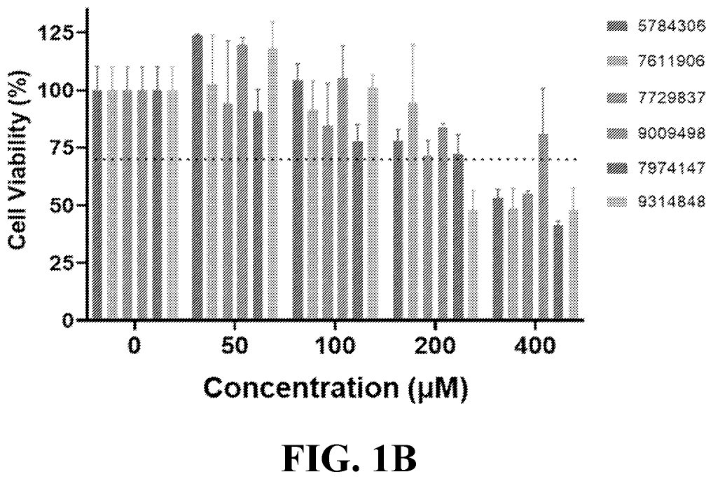

To explore this possibility further, Z′-factor testing was performed to determine whether an appropriate ciprofloxacin concentration could be identified that reproducibly allowed growth of wild type (PBP4+) cells, but inhibited growth of PBP4− cells, in a high throughput setting. Growth measures were performed for wild type and Δpbp4 mutant cells in media alone or media supplemented with 1.0, 2.0, 3.0 or 4.0 μg/mL ciprofloxacin. Media containing 2 μg/mL ciprofloxacin provided the most reliable culture conditions to distinguish between wildtype cells and the Δpbp4 strain, generating a Z′-factor score of 0.31 (FIG. 1A).

Screening for Small Molecule S. aureus PBP4 Inhibitors

Members of a 30,000 compound ChemBridge® small molecule diversity set were individually screened at 50 μM to identify molecules that reduced wild type USA300 growth to that of the Δpbp4 strain in media supplemented with 2.0 μg/mL ciprofloxacin. Results revealed that 29,679 of these molecules (98.9%) did not impact the organism's growth in the presence of ciprofloxacin, whereas 321 compounds (1.1%) eliminated growth. The growth inhibiting properties of the 321 compounds may be due to either: 1) direct inhibition of PBP4 function, or 2) standalone antimicrobial activity. To enrich for PBP4 inhibitors, the standalone antimicrobial activity of each of the 321 compounds was directly evaluated in the absence of antibiotic. A total of 160 compounds (49.8%) inhibited USA300 growth at 50 μM and, because S. aureus PBP4 is non-essential, were removed from further consideration as PBP4-specific inhibitors. Conversely, 161 compounds (50.2%) exhibited no detectible standalone antimicrobial activity, suggesting a subset of these molecules may include PBP4-specific inhibitors.

To distinguish putative PBP4 inhibitors from non-specific antibiotic potentiators the remaining 161 compounds evaluated for potentiation with another antibiotic, mupirocin. As shown in Table 1, there was no detectible difference in mupirocin susceptibility between wild type USA300 cells and pbp4 mutant cells, suggesting that PBP4 inhibitors should have no impact on mupirocin susceptibility, whereas non-specific antibiotic potentiators may alter the mupirocin activity. Standard fractional inhibitory concentration (FIC) testing to measure compound interactions of all 161 compounds revealed that 65 (40.4%) potentiated the antimicrobial effects of mupirocin toward wild type USA300 cells indicating that they are non-specific antibiotic potentiators and were triaged, whereas 96 compounds (59.6%) had no impact on the strain's susceptibility to mupirocin. Next, an early test of potential eukaryotic cell cytotoxicity was performed by measuring the effects of each of the 96 compounds for growth inhibition against yeast cells. Two compounds (˜2%) inhibited Saccharomyces cerevisiae growth, and thus were considered likely to also be toxic toward mammalian cells and triaged, whereas the remaining 94 compounds (˜98%) had no effect. Taken together, these 94 molecules represent a compound set that is likely to be enriched for S. aureus PBP4 inhibitors worthy of follow-on characterization.

### Potentiation of β-Lactams

Prioritized compounds were tested for their ability to reverse PBP4 mediated antibiotic resistance in another S. aureus background using three isogenic strains that became available during the study (generous gift from Dr. S. Chatterjee; University of Maryland). These strains included: 1) COLnex, which is a methicillin susceptible strain, 2) CRB, a methicillin/ceftobiprole resistant strain of COLnex that overproduces PBP4, and 3) CRBΔpbp4, a methicillin susceptible strain of CRB lacking the pbp4 gene. MIC testing (Table 1) confirmed the resistance profiles of each of these strains to ceftobiprole as being 0.25 μg/mL (COLnex), 128 μg/mL (CRB; PBP4-overproducer) and 0.25 μg/mL (CRBΔpbp4), as previously reported. It was reasoned that the observed 512-fold difference in ceftobiprole resistance between the PBP4-overexpressor and corresponding Δpbp4 derivative provided an excellent dynamic range to further evaluate whether the putative inhibitors do (or do not) display the ability to reverse PBP4 associated 1-lactam resistance in a second strain background. Of note, while PBP4 appeared to modulate fluoroquinolone resistance in the USA300 strain used here, the presence or absence of the pbp4 gene did not appear to significantly impact fluoroquinolone resistance in the COLnex strain set (Table 1).

Standard fractional inhibitory concentration testing was performed using S. aureus strain CRB in checkerboard format in which each row contained increasing concentrations of ceftobiprole (0, 1.0, 2.0, 4.0, 8.0, 16, 32, 64, or 128 μg/ml) and each column contained increasing concentrations (0, 3.125, 6.25, 12.5, 25, 50, or 100 μM) of a putative PBP4 inhibitor. Of the 94 compounds evaluated, 88 (93.6%) either did not, or only marginally reduced, CRB ceftobiprole susceptibility (2-fold MIC reduction at 100 μM putative PBP4 inhibitor), suggesting that they are not able to inhibit the strain's PBP4 or are low potency inhibitors (data not shown). However, six compounds exhibited either additive or synergistic antimicrobial activity (FICI≤1) in combination with ceftobiprole, suggesting that they may be effective PBP4 inhibitors (Table 2). More specifically, while CRB growth was not affected by 128 μg mL⁻¹ ceftobiprole (alone) or ≥100 μM each compound (alone), the strain's ceftobiprole MIC decreased from 128 to 4.0-64 μg/mL when combined with 12.5 to 50 μM each putative PBP4 inhibitor. Compound 5784306 reduced ceftobiprole resistance 2-fold, compound 7611906 reduced resistance 8-fold, compounds 9009498, 7974147, and 9314848 reduced resistance 16-fold, and compound 5784306 reduced resistance 32-fold (all compound numbers refer to ChemBridge® chemical identifiers). To establish whether these compound-associated reductions in strain CRB ceftobiprole resistance were PBP4 dependent, FIC testing was repeated using strain CRBΔpbp4 (Table 2). Four compounds had no impact on ceftobiprole activity toward the deletion strain, whereas one compound provided a modest 2-fold decrease in resistance, suggesting their ability to reduce CRB ceftobiprole resistance was primarily due to PBP4 inhibition. Similarly, while compound 9009498 decreased ceftobiprole resistance of the pbp4 mutant strain 4-fold, the compound reduced resistance of wild type USA300 16-fold, indicating that the compound affects PBP4-mediated resistance. Thus, all compounds were carried forward for further characterization.

TABLE 2
Antimicrobial effects of ceftobiprole and putative PBP4 inhibitors.

| | CRB | | | | | CRBΔpbp4 | | | | |
|---|---|---|---|---|---|---|---|---|---|---|
| | Alone | | Combination | | | Alone | | Combination | | |
| Compound | BPR¹ | Cmpd² | BPR¹ | Cmpd² | Fold³ | BPR¹ | Cmpd² | BPR¹ | Cmpd² | Fold³ |
| 5784306 | 128 | 100 | 4 | 12.5 | 32 | 0.25 | 400 | 0.25 | 12.5 | 0 |
| 7611906 | 128 | 400 | 16 | 25 | 8 | 0.25 | 400 | 0.25 | 25 | 0 |
| 7729837 | 128 | 200 | 64 | 50 | 2 | 0.25 | 400 | 0.25 | 50 | 0 |
| 9009498 | 128 | 100 | 8 | 25 | 16 | 0.25 | 100 | 0.06 | 25 | 4 |
| 7974147 | 128 | 400 | 8 | 25 | 16 | 0.25 | 400 | 0.25 | 25 | 0 |
| 9314848 | 128 | 400 | 8 | 25 | 16 | 0.25 | 400 | 0.125 | 25 | 2 |

¹BPR (ceftobiprole) μg/ml;
²Cmpd (Putative PBP4 inhibitor) μM;
³Fold-decrease in ceftobiprole resistance when in combination with the indicated putative PBP4 inhibitor.

### Human Cell Cytotoxicity Measures of Putative PBP4 Inhibitors

Although none of the six putative PBP4 inhibitors affected the growth of yeast cells, further evaluation of the inhibitors was performed. Specifically, the cytotoxicity toward human cells was performed. To do so, standard MTT assays were performed using human HepG2 hepatocellular carcinoma cells in the presence of 0, 50, 100, 200, and 400 μM of each compound. Following International Organization for Standardization guidelines, compounds that resulted in cell viability below 70% were considered to exhibit human cell cytotoxicity. As shown in FIG. 1B, the six compounds were not toxic to HepG2 cells at ≤100 μM of the compound, which is at least 2× concentration required to reduce strain CRB PBP4-associated ceftobiprole resistance.

### Effects of Putative Inhibitors on PBP4 Expression

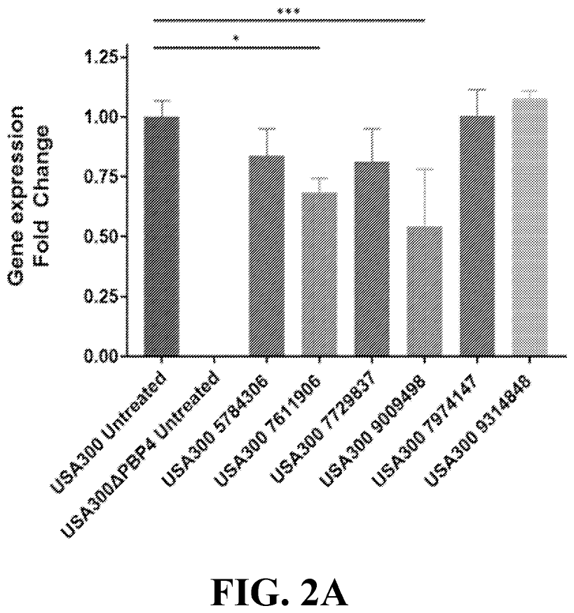

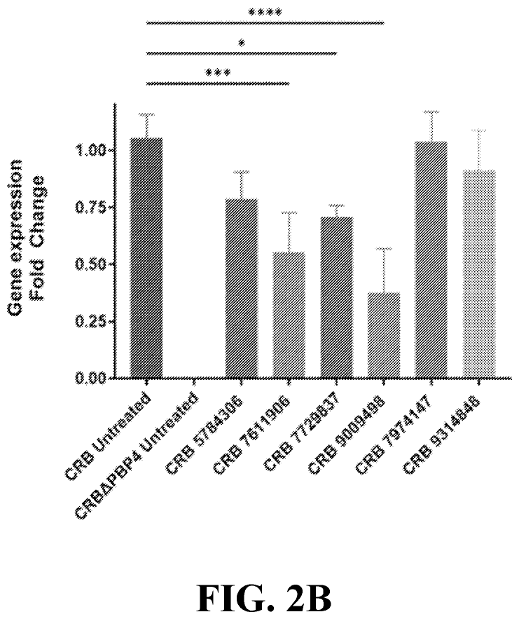

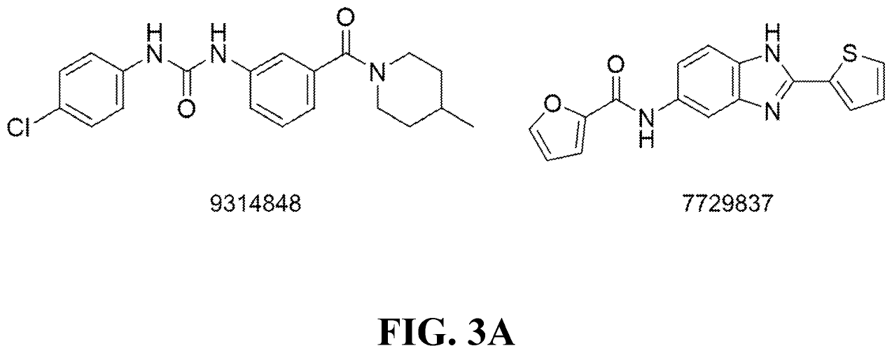

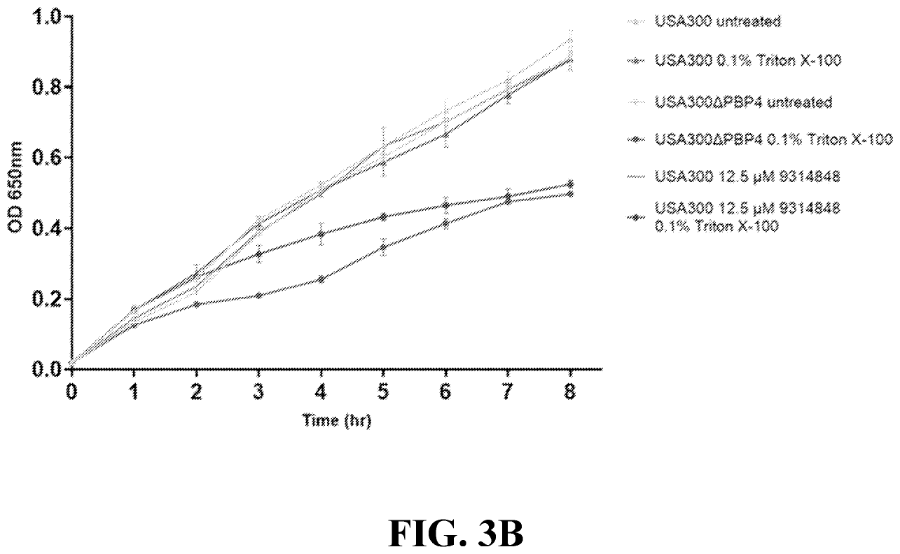

Given that each of the six compounds appeared to affect PBP4-mediated antibiotic resistance, further experiments evaluated whether function was related to inhibition of the protein's function or reduction of pbp4 expression, the latter of which may be readily overcome by regulatory mutations that could result in resistance. Thus, the intent was to prioritize compounds that may affect the protein's function and de-prioritize compounds that affect the protein's expression. To evaluate the effect of the compounds on protein express, quantitative real-time reverse transcriptase PCR (qRT-PCR) was used to measure the pbp4 transcript titers within USA300 and CRB cells following compound treatment (FIG. 2A-B). Three compounds reduced pbp4 expression in both CRB and USA300, whereas one compound (77295377) slightly reduced USA300 pbp4 expression but significantly reduced expression in CRB. In contrast, two compounds, 7974147 and 9314848, did not affect pbp4 expression, suggesting that they may directly interact with the protein. The compounds 9314848 (FIG. 3A) was prioritized because the molecule appeared the more druggable and chemically tractable for downstream structure activity relationship studies.

### Effect of PBP4 Inhibitors on Triton X-100 Susceptibility

It has been established that S. aureus tolerance to Triton X-100 is mediated by PBP4. More directly, wild type cells are tolerant of 0.1% of the detergent, while pbp4 mutant cells are not. Accordingly, to test as whether 9314848 may affect PBP4's cellular function, evaluation of whether compound treated wild type cells phenocopy the growth defect of pbp4 mutant cells in the presence of 0.1% triton X-100 (FIG. 3B). The wild type USA300 displayed a robust growth phenotype in the absence or presence of the detergent, whereas pbp4 mutant cells displayed wild type growth in the absence of the detergent and a growth defect in media supplemented with 0.1% Triton X-100. Similarly, 9314848 treated wildtype cells did not display a growth defect in the absence of detergent but exhibited growth defect in the presence of Triton X-100, recapitulating the growth characteristics of pbp4 mutant cells in the presence of detergent.

### Ability of PBP4 Inhibitors to Bind to PBP4 or PBP2A

To more directly evaluate whether 9314848 is likely to affect PBP4 function, the compound's ability to bind recombinant PBP4 was measured using BOCILLIN™ FL fluorescence polarization displacement assays. The premise for the assay is that the fluorescence polarization of the fluorescently labeled β-lactam, penicillin V (BOCILLIN™ FL), will increase upon binding to its cognate target (i.e., PBP4). Further, compounds that bind PBP4 and inhibit its function would impede BOCILLIN™ FL binding thereby allowing assessment of the protein binding affinity of 9314848 using a simple fluorescence reduction assay.

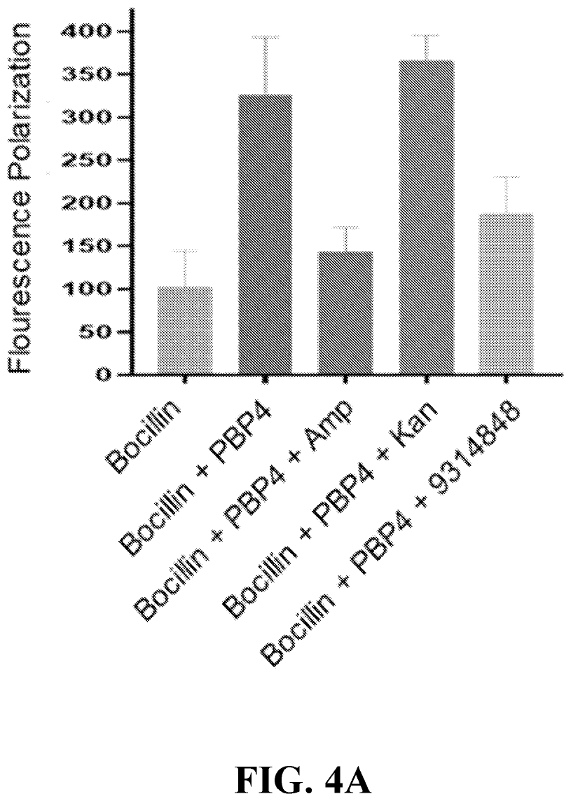

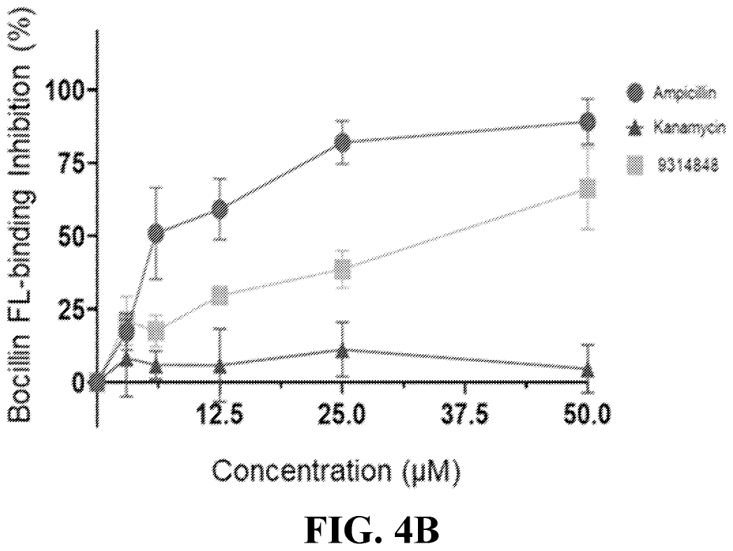

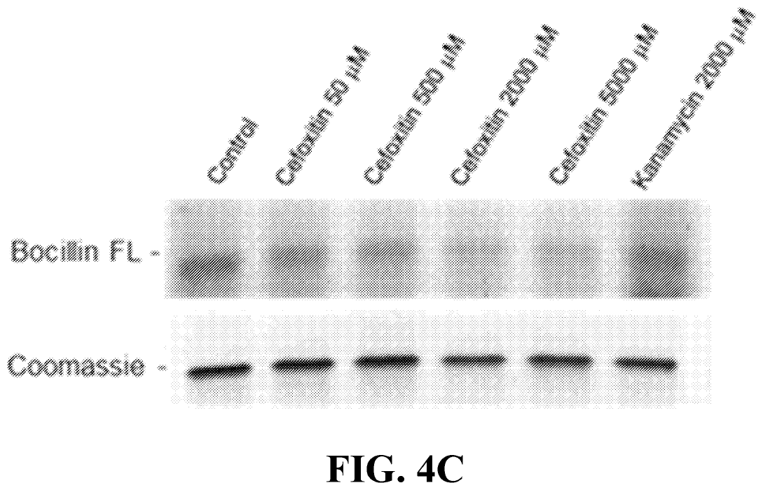

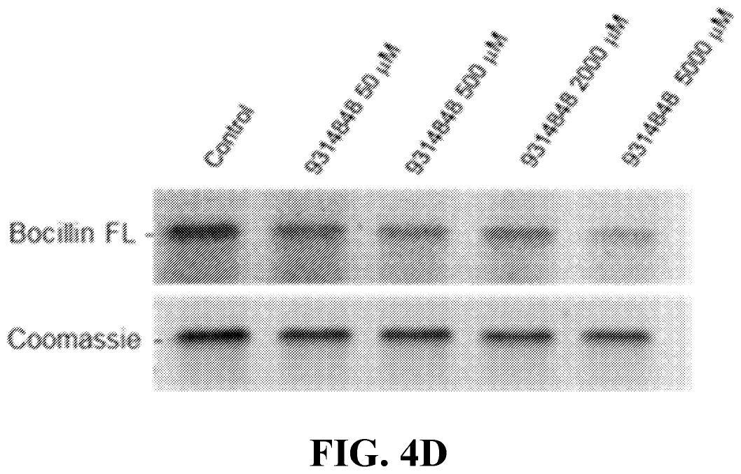

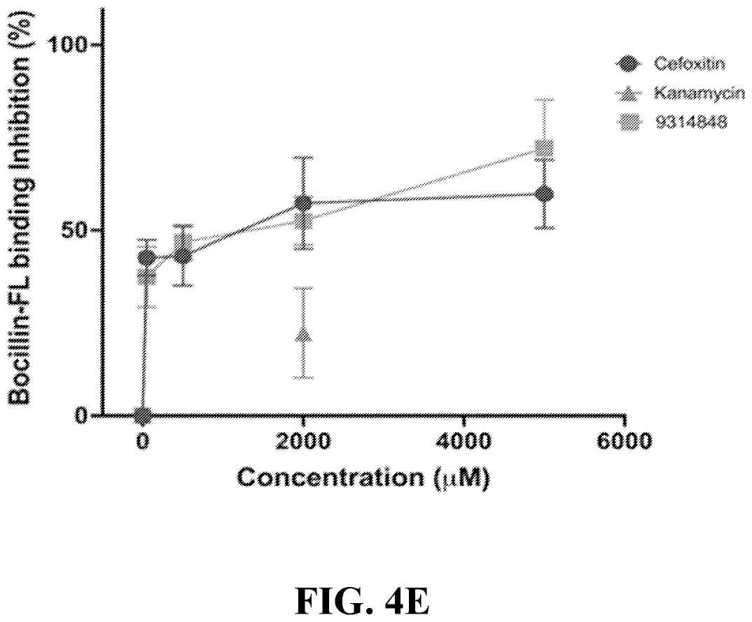

As shown in FIG. 4A, control reactions showed that BOCILLIN™ FL (alone) exhibited relatively low fluorescence polarization (˜100 relative fluorescent units; RFUs) but increased 3-fold following incubation with recombinant Bacillus subtilis PBP4 (310 RFU). To determine whether the fluorescence polarization assay was able to measure the binding properties of a compound known to bind PBP4 (positive-control) vs. a compound not expected to bind the protein (negative-control), PBP4 was preincubated with either the β-lactam ampicillin (positive-control), or the aminoglycoside kanamycin (negative-control). Preincubation with ampicillin, which covalently binds the PBP4 active site, limited BOCILLIN™ FL binding. Conversely, preincubation with kanamycin (negative-control) had no impact on BOCILLIN™ FL binding to PBP4, resulting in a high fluorescent polarization signal. Tests of 9314848 in which the compound was preincubated with PBP4 followed by the addition of BOCILLIN™ FL revealed that the compound limited fluorescence polarization, indicating that 9314848 may be either a competitive inhibitor or and allosteric modulators that binds in an allosteric site and alters PBP4 architecture in a manner that inhibits BOCILLIN™ FL binding.

A subsequent dose-response study was conducted (FIG. 4B) in which PBP4 was preincubated with 0, 3.125, 6.25, 12.5, 25, or 50 μM of ampicillin, kanamycin, or 9314848. Binding experiments with ampicillin exhibited a smooth PBP4 dose-response binding curve reaching maximum BOCILLIN™ FL binding inhibition at 25-50 μM, whereas kanamycin had no inhibitory effect on PBP4 BOCILLIN™ FL binding. Binding of U.S. Pat. No. 9,314,848 to PBP4 was observed at 3.125 μM and 6.25 μM; however increased binding was observed at all concentrations above 6.25 μM. At 50 μM, 9314848 reduced BOCILLIN™ FL binding by ˜66%. Based upon this data, the IC₅₀ value of 9314828 was determined to be 13.4 μM. As a control, the ability of 50 μM 9314848 to bind to PBP2A was evaluated, and it was observed that the compound displayed low and inconsistent inhibition of BOCILLIN™ FL labeling of PBP2A (25.81±11.41%).

A second, approach was used to verify the impact of 9314848 on PBP4 substrate binding in which the protein was preincubated with compound or control antibiotics then labeled with the BOCILLIN™ FL and resulting fluorescent labeled protein was visualized following gel electrophoresis. As expected, in the absence of compound BOCILLIN™ FL readily labeled recombinant PBP4, whereas preincubation of the protein with the β-lactam antibiotics ampicillin (not shown) or cefoxitin reduced the protein's ability to bind the fluorescent substrate in a dose-dependent manner (FIG. 4C and FIG. 4E). Conversely, preincubation of PBP4 with 2000 μM or 5000 μM (not shown) kanamycin did not significantly impact the protein's ability to bind BOCILLIN™ FL. As shown in FIG. 4D and FIG. 4E, dose response testing in which PBP4 was preincubated with 0, 50, 500, 2000, or 5000 μM of 9314848 revealed that the compound limited PBP4 BOCILLIN™ FL binding by 37.50±8.20% (50 μM) to 72.12±13.09% (5000 PM). Of note, treatment of PBP4 with ampicillin, cefoxitin, or 9314848 at concentrations below 50 μM did not reproducibly demonstrate BOCILLIN™ FL binding inhibition.

### PBP4 Inhibitors Prevent Propagation in μSiM-CA Canaliculi Model

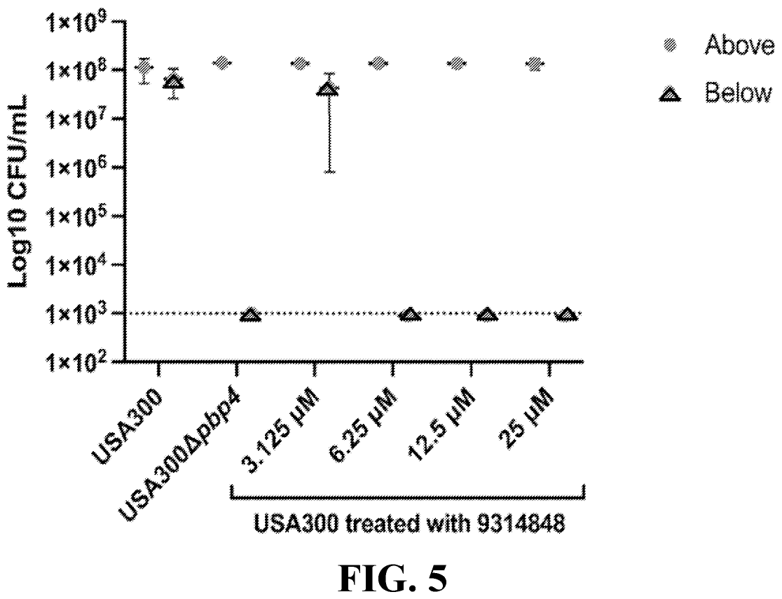

Subsequent studies evaluated whether 9314848 could also inhibit PBP4-mediated S. aureus canalicular bacterial colonization. PBP4 has been shown to be involved in an organism's transmigration through μSiM-CA devices that mimic the canalicular network orifice. Thus, 9314848 was evaluated for its ability to eliminate S. aureus migration through μSiM-CA membrane pores. As shown in FIG. 5, after inoculation of 1×10⁷ CFUs of wild type USA300 to the top well of μSiM-CA devices, the bacteria readily migrated through the 500 nm pores, resulting in virtually an equivalent number of cells in the device bottom chamber. Conversely, the USA300Δpbp4 strain was unable to efficiently transverse the membrane, verifying the requirement of PBP4 for S. aureus membrane transmigration. To evaluate whether 9314848 could eliminate PBP4-associated S. aureus μSiM-CA membrane migration, 1×10⁷ CFU wild type cells were incubated with 0, 3.125, 6.25, 12.5, or 25 μM of the compound for 2 hours and applied to the top chamber of the device. Following incubation, cells in the top and bottom chambers were enumerated. Compound treatment did not affect the viability of cells within the top/treatment chamber. Further, while treatment with ≤3.125 μM of the molecule had no effect on S. aureus membrane transmigration, concentrations greater than 6.25 μM (2.5 μg/ml) completely inhibited the process, suggesting the EC₅₀ is between 3.125 and 6.25 μM.

### Conclusions

While PBP2a has historically been considered the main driver of MRSA, the recent finding that high-level S. aureus-lactam resistance occurs in strains not capable of producing PBP2a has led to investigation of non-canonical pathway(s) of MRSA development, as well as fundamental questions regarding the clinical diagnostic practices that rely on detection of mecA, the gene encoding PBP2a, as a MRSA determinant. Recent studies have revealed that development of PBP2a-independent MRSA is associated with PBP4 production in community-acquired MRSA isolates and/or overproduction of a PBP4 variant within the hospital-acquired strain COL background. With regard to the latter, the COL derivative, CRB, is a MRSA strain that lacks mecA but contains a 36-base pair duplication upstream of the PBP4 open reading frame and two active site substitutions (E183A and F241R) that are thought to play a central role in conferring the strain's MRSA phenotype. The PBP4 missense mutations seem to impair binding of ceftobiprole approximately 150-fold, whereas the upstream mutation is thought to lead to an approximately 145-fold increase in PBP4 production, which in-turn elicits ceftaroline resistance. Thus, the combined activity of both overproduction of a PBP4-derivative produces resistance to both antibiotics as well as other β-lactams within CRB. Agents that limit PBP4 activity and/or the PBP4 variant within strain CRB could reverse the PBP2a-independent pathway of MRSA development. Such agents may have therapeutic value as adjuvants dosed concurrently with 3-lactams that are effective against PBP2a, such as ceftobiprole.

In addition to modulating β-lactam resistance, PBP4 may be a previously overlooked S. aureus virulence factor that plays a role in osteomyelitis pathogenesis, a debilitating musculoskeletal infection with a prevalence of approximately 22 cases per 100,000 person-years. More directly, in a study designed to determine the bacterial source of re-occurring osteomyelitis, it was found that S. aureus has the remarkable ability to invade and colonize cortical bone osteocyte lacuno-canalicular networks (OLCN), which is hypothesized to provide a reservoir for the organism to cause persistent infection. PBP4 was subsequently determined to be essential for OLCN invasion, both in an in vitro (μSiM-CA) model system and in animals, providing a promising target for therapeutic development to combat osteomyelitis. Further, cells lacking PBP4 expression have less cross-linked peptidoglycan and decreased cell wall stiffness, suggesting that cell wall metabolism plays a key role in the organism's ability to invade sub-micron canaliculi. Recently, μSiM-CA and follow-on animal studies have recently revealed that another penicillin binding protein, PBP3, which interfaces with RodA to mediate S. aureus side-wall peptidoglycan synthesis, is also required for OLCN invasion and bacterial colonization. Thus, agents that inhibit PBP4 and/or PBP3 function may represent valuable therapeutics that reduce S. aureus' ability to cause reoccurring osteomyelitis; however, PBP3 is not associated with high-level β-lactam resistance.

In the current study a screening approach was developed to identify small molecule inhibitors of S. aureus PBP4, based on the premise that such agents may have dual therapeutic utility that can reverse β-lactam resistance and/or reduce osteomyelitis pathogenesis. To that end, it has been previously shown that cefoxitin, a β-lactam that binds PBP4 with high affinity, reversed the PBP4 mediated oxacillin resistance phenotype of community acquired MRSA strains. Using a MRSA USA300 strain as a model screening organism it was observed that PBP4 modulates the organism's β-lactam resistance phenotype, but that the protein was associated with greater resistance to the fluoroquinolone, ciprofloxacin, which was an unexpected finding that has not been previously reported. Nonetheless, others have found that PBP4 mediates Erwinia sp. Fluoroquinolone resistance. Presumably, the previously observed decrease in peptidoglycan crosslinking of PBP4 deficient cells facilitates the antibiotic's cellular entry, although this was not formally evaluated and was not a generalizable phenotype for other antibiotic classes, such as mupirocin, that target cytoplasmic enzymes.

A screen of 30,000 compounds identified agents that potentiated the antimicrobial activity of ciprofloxacin toward wild type USA300, and also potentiated the activity of nafcillin toward the strain (data not shown) but had no impact on the antimicrobial activity of mupirocin, effectively phenocopying a USA300Δpbp4 strain. Follow-on studies performed using CRB (PBP4-variant over-expresser) determined that a subset of compounds also reduced the strain's MRSA phenotype. While CRB was resistant to 128 μg/mL of ceftobiprole, resistance decreased to 4-16 μg/mL in the presence of 12.5-25 μM each compound. Three compounds limited S. aureus pbp4 transcription, providing an indication that the screening paradigm effectively identifies agents that modulate PBP4 activity either directly or indirectly; identifying the cellular targets of these three compounds is expected to provide insight regarding the regulatory networks that modulate S. aureus PBP4 expression. Two compounds, 7974147 and 9314848 did not significantly impact pbp4 transcription, suggesting that they may affect the protein's function.

For the current study, attention was focused on characterizing compound 9314848, which reduced strain USA300 ceftobiprole resistance from 1 μg/mL to 0.25 μg/mL and strain CRB ceftobiprole resistance from 128 μg/mL to 8 μg/mL and appeared to be S. aureus specific, as the compound had no effect on Enterococcus faecalis or Pseudomonas aeruginosa ceftobiprole susceptibility (not shown). To further evaluate whether the compound affects PBP4 activity a series of assays were performed. First, Triton X-100 studies indicated that 9314848 increases wild type USA300 susceptibility to the detergent to a level also observed with USA304270Δpbp4 cells. Second, BOCILLIN™ FL fluorescence polarization studies indicated that the compound was capable of binding recombinant B. subtilis PBP4 protein. Third, using μSiM-CA devices, the compound appeared to limit S. aureus membrane transmigration. From these perspectives, 9314848 appears to limit S. aureus PBP4-associated resistance and migration through devices mimicking cortical bone OLCN, suggesting that the molecule may represent a promising chemical scaffold for advancing to medicinal chemistry-based optimization and refinement.

### Example 2: Phenyl-Urea Based Small Molecule Adjuvants Targeting PBP4

### Abbreviations

DCM for dichloromethane;
DMF is N,N-dimethylformamide;
EDC is 1-ethyl-3-(3-dimethylaminopropyl)carbodiimide;
DMAP is 4-dimethylaminopyridine
MeOH is methanol;
Pd/C is palladium on carbon;
N₂S₂O₄ is sodium dithionite;
CDI is carbonyldiimidazole,
TEA is triethylamine;
TLC is thin-layer chromatography;
EtOAc is ethyl acetate;
HPLC is high-performance liquid chromatography;
TFA is trifluoracetic acid;
min or min. is minute(s);
h or hr. is hour(s);
rt, RT, or r.t. is room temperature;
and sat. is saturated

### Synthesis of Compounds

### Synthesis of Example Intermediates

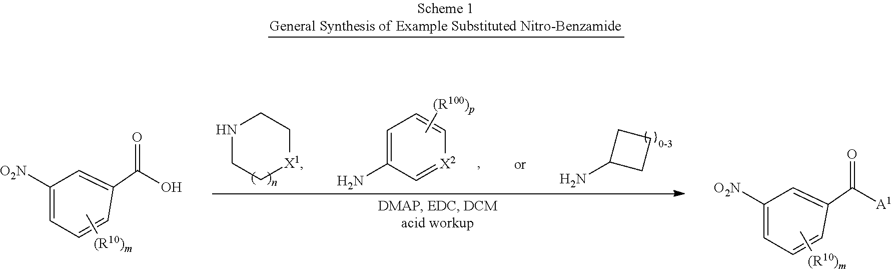

An oven-dried 100-mL round-bottomed flask equipped with a magnetic stirring bar, and an N₂ inlet was charged with the nitrobenzoic acid (5.98 mmol), DMAP (332 mg, 2.72 mmol) and EDC (3.1 g, 16.32 mmol). The flask was put under vacuum and purged with argon. To the reaction mixture, 26 mL anhydrous DCM was added and stirred for 30 minutes. To the reaction mixture, an amine containing compound (5.44 mmol) was then added and allowed to stir overnight. The resulting mixture was diluted with DCM and washed with 2 M HCl (1×8 mL), water (3×10 mL), dried with MgSO₄, and concentrated in vacuo to provide the title compound.

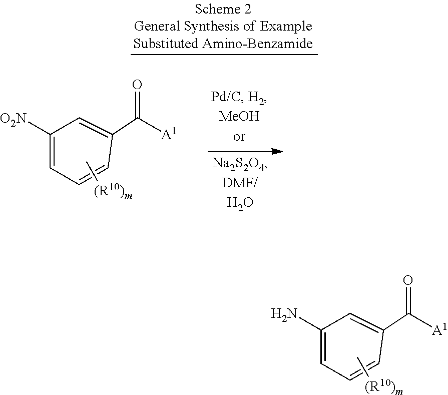

A 100-mL round-bottomed flask was equipped with a magnetic stirring bar and charged with Pd/C 50% wet (10.7 mg, 0.1 mmol). The flask was purged with H₂ and the nitrobenzamide (2.0 mmol) was added dissolved in methanol. The reaction mixture was stirred at room temperature until completion and monitored via TLC. The mixture was filtered and concentrated to isolate the aminobenzamide. In an alternative experimental procedure, a 100-mL round-bottomed flask was equipped with a magnetic stirring bar and charged with sodium dithionite (1.23 g, 7.05 mmol). To the reaction flask, a solution of DMF/H₂O (9:1, 40 mL) and nitrobenzamide (2.0 mmol) was added. The reaction mixture was heated to 50° C. and let stirred until completion (5 h to overnight). The mixture was diluted with water, extracted with EtOAc (3×15 mL), the organic layers combined, dried with MgSO₄, and concentrated. Purification of the residue by chromatography on silica gel with EtOAc/Hex for elution provided the title compound.

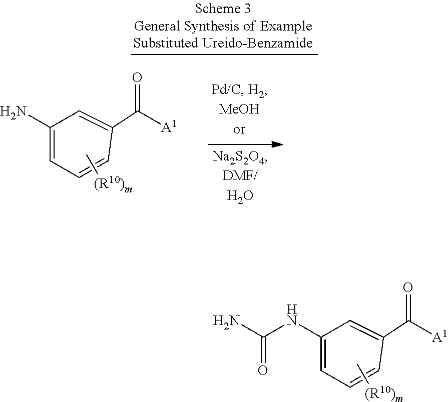

To an oven-dried 50-mL round-bottomed flask, an ureido-benzamide (0.46 mmol) was added. To the reaction flask, 20 mL of anhydrous DCM and the appropriate isocyanate were added (0.46 mmol) and stirred. The reaction was reacted overnight. The resulting mixture was diluted with DCM, washed with water (3×10 mL), and washed with brine (2×10 mL). The organic layers were combined, dried with MgSO₄, and concentrated in vacuo. The resulting residue was purified via column chromatography on silica gel using EtOAc/hexanes for elution or by recrystallization in DCM. If necessary, this was followed by HPLC using the gradient 20-80% acetonitrile and water with 0.1% TFA to provide the title compound.

### Synthesis of Example Final Compounds

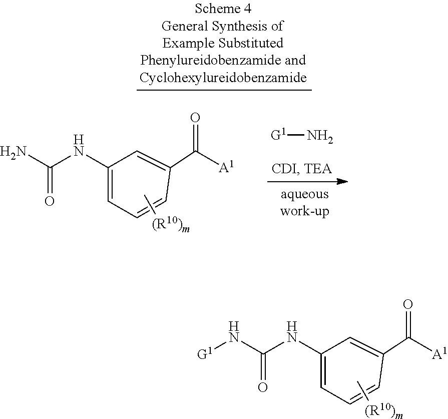

To an oven-dried 50-mL round-bottomed flask, the resulting amine from above (0.46 mmol) was added. To the reaction flask, 20 mL of anhydrous DCM and TEA (92.7 mg, 0.916 mmol) were added. The reaction mixture was stirred at room temperature for 5 minutes. CDI (111 mg, 0.687 mmol) was added to the mixture and stirred until consumption of the starting material was complete. The appropriate amine was then added to the reaction mixture and stirred at room temperature overnight. The mixture was diluted with DCM, washed with water (3×10 mL), dried with MgSO₄, and concentrated in vacuco. The resulting residue was purified via column chromatography on silica gel using EtOAc/hexanes or DCM/MeOH for elution or by recrystallization in DCM. If necessary, this was followed by HPLC using the gradient 20-80% acetonitrile and water with 0.1% TFA to provide the title compound.

### Example Compounds

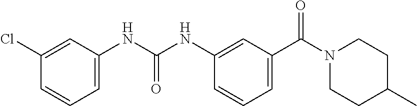 [SMILES: CC1CCN(C(=O)c2cccc(NC(=O)Nc3cccc(Cl)c3)c2)CC1]

1-(4-Chlorophenyl)-3-(3-(4-methylpiperidine-1-carbonyl)phenyl)urea (2). Compound 2 was purchased from Aronis Compounds.

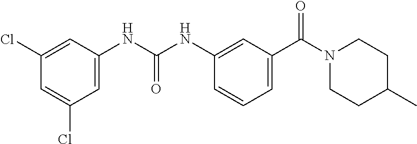 [SMILES: CC1CCN(C(=O)c2cccc(NC(=O)Nc3cc(Cl)cc(Cl)c3)c2)CC1]

1-(3,5-Dichlorophenyl)-3-(3-(4-methylpiperidine-1-carbonyl)phenyl)urea (3). Using the general procedures outlined above, 3 was synthesized as a yellow solid in 60% yield (112 mg, 0.28 mmol). ¹H NMR (400 MHz, CDCl3) δ 8.34 (s, 1H), 8.1 (s, 1H), 7.67 (d, J=8.8 Hz, 1H), 7.50 (s, 1H), 7.35 (t, J=9.8 Hz, 9.6 1H), 6.18 (d, J=9.6 Hz, 1H), 4.73 (d, J=6.1 Hz, 1H), 4.66 (d, J=15 Hz, 1H), 3.73 (d, J=15.7 Hz, 1H), 3.05 (t, J=5.15, 9.9 Hz, 1H), 3.28 (t, J=15.8, 14.5 Hz, 1H), 1.65 (m, 3H), 1.23 (m, 2H), 0.98 (s, 3H).

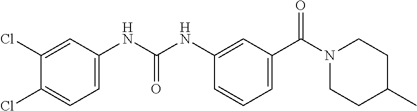 [SMILES: CC1CCN(C(=O)c2cccc(NC(=O)Nc3ccc(Cl)c(Cl)c3)c2)CC1]

1-(3,4-Dichlorophenyl)-3-(3-(4-methylpiperidine-1-carbonyl)phenyl)urea (4). Using the general procedures outlined above, 4 was synthesized as a white solid in 60% yield (112 mg, 0.28 mmol). ¹H NMR (400 MHz, DMSO-d6) δ 7.88 (s, 1H), 7.52 (d, J=8.05 Hz, 1H), 7.43 (d, J=10.20 Hz, 2H), 7.26 (t, J=5.70, 3.45, Hz, 2H), 6.96 (d, J=9.40 Hz, 1H), 4.43 (d, J=12.95 Hz, 1H), 3.56 (m, 1H), 3.00 (m, 1H), 2.67 (t, J=2.25, 2.25 Hz, 1H), 1.63 (m, 3H), 1.06 (m, 2H), 0.92 (s, 3H).

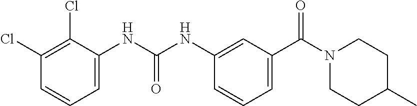 [SMILES: CC1CCN(C(=O)c2cccc(NC(=O)Nc3cccc(Cl)c3Cl)c2)CC1]

1-(2,3-Dichlorophenyl)-3-(3-(4-methylpiperidine-1-carbonyl)phenyl)urea (5). Using the general procedures outlined above, 5 was synthesized as a yellow solid in 53% yield (98 mg, 0.24 mmol). ¹H NMR (400 MHz, Methanol-d4) δ 8.17 (d, J=7.9 Hz, 1H), 7.64 (s, 1H), 7.51 (d, J=9.1 Hz, 2H), 7.41 (t, J=9.6, 10 Hz, 1H), 7.25 (m, 2H), 7.07 (d, J=9.5 Hz, 1H), 4.62 (d, J=15.6 Hz, 1H), 3.77 (d, J=16.3 Hz, 1H), 3.12 (t, J=15, 13.4 Hz, 1H), 2.86 (t, J=15.1, 14.3 Hz, 1H), 1.71 (m, 3H), 1.21 (m, 2H), 1.02 (s, 3H).

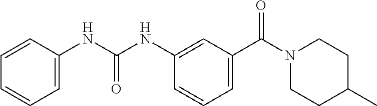 [SMILES: CC1CCN(C(=O)c2cccc(NC(=O)Nc3ccccc3)c2)CC1]

1-(3-(4-Methylpiperidine-1-carbonyl)phenyl)-3-phenylurea (6). Compound 6 was purchased from Aronis Compounds.

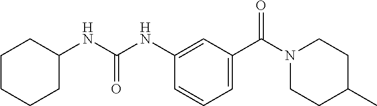 [SMILES: CC1CCN(C(=O)c2cccc(NC(=O)NC3CCCCC3)c2)CC1]

1-Cyclohexyl-3-(3-(4-methylpiperidine-1-carbonyl)phenyl)urea (7). Using the general procedures outlined above, 7 was synthesized as a yellow solid in 35% yield (67 mg, 0.20 mmol). ¹H NMR (400 MHz, Methanol-d4) δ 7.51 (s, 1H), 7.37 (m, 2H), 6.68 (d, J=9.00 Hz, 1H), 4.60 (d, J=14.9 Hz, 1H), 3.75 (d, J=16.05 Hz, 1H), 3.58 (m, 1H), 3.09 (m, 1H), 2.85 (m, 1H), 1.93 (m, 2H), 1.74 (m, 6H), 1.45 (m, 2H), 1.25 (m, 5H), 1.01 (s, 3H).

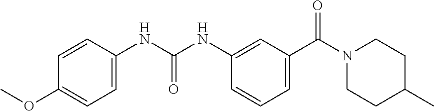

1-(4-Methoxyphenyl)-3-(3-(4-methylpiperidine-1-carbonyl)phenyl)urea (8). Using the general procedures outlined above, 8 was synthesized as a yellow solid in 34% yield (56 mg, 0.16 mmol). ¹H NMR (400 MHz, Methanol-d4) δ 7.61 (dd, J=4.15, 2.10 Hz, 2H), 7.51 (dd, J=9.10, 1.15 Hz, 2H), 7.40 (t, J=9.65, 9.95 Hz, 2H), 7.05 (d, J=9.45 Hz, 2H), 4.62 (d, J=15.00 Hz, 1H), 3.77 (d, J=15.85 Hz, 1H), 3.15 (t, J=15.7, 15.1 Hz, 1H), 2.86 (t, J=15.95, 14.4 Hz, 1H), 1.91 (s, 3H), 1.68 (m, 2H), 1.19 (m, 3H), 1.00 (s, 3H).

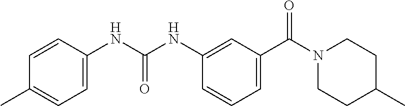

1-(4-Methylphenyl)-3-(3-(4-methylpiperidine-1-carbonyl)phenyl)urea (9). Using the general procedures outlined above, 9 was synthesized as a white solid in 59% yield (96 mg, 0.27 mmol). ¹H NMR (400 MHz, Methanol-d4) δ 7.47 (dd, J=10.25, 1.35 Hz, 1H), 7.38 (t, J=9.75, 9.95 Hz, 1H), 7.30 (dd, J=8.15, 8.1 Hz, 3H), 7.12 (dd, J=7.55, 7.70 Hz, 2H), 7.03 (d, J=9.45 Hz, 1H), 4.61 (d, J=15.00 Hz, 1H), 3.78 (d, J=15.15 Hz, 1H), 3.15 (t, J=17.0, 13.9 Hz, 1H), 2.89 (t, J=15.57, 15.8 Hz, 1H), 2.30 (s, 3H), 1.69 (m, 3H), 1.20 (m, 2H), 1.02 (s, 3H).

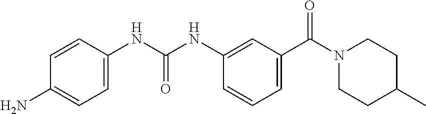 [SMILES: CC1CCN(C(=O)c2cccc(NC(=O)Nc3ccc(N)cc3)c2)CC1]

1-(4-Aminophenyl)-3-(3-(4-methylpiperidine-1-carbonyl)phenyl)urea (10). Using the general procedures outline above, 10 was synthesized as a white solid in 91% yield (47 mg, 0.13 mmol). ¹H NMR (400 MHz, DMSO-d6) δ 7.46 (s, 1H), 7.43 (d, J=10.28 Hz, 2H), 7.35 (d, J=8.24 Hz, 1H), 7.26 (t, J=7.92, 7.68, Hz, 1H), 7.10 (m, Hz, 2H), 6.87 (d, J=7.48 Hz, 1H), 4.27 (d, J=9.12 Hz, 1H), 3.51 (d, J=8.56, Hz, 1H), 2.92 (d, J=9.56, Hz, 1H), 2.68 (d, J=9.12 Hz, 1H), 1.56 (m, 3H), 1.00 (m, 2H), 0.86 (d, J=9.16 Hz, 3H).

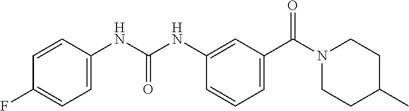 [SMILES: CC1CCN(C(=O)c2cccc(NC(=O)Nc3ccc(F)cc3)c2)CC1]

1-(4-Fluorophenyl)-3-(3-(4-methylpiperidine-1-carbonyl)phenyl)urea (11). Using the general procedures outlined above, 11 was synthesized as a yellow solid in 51% yield (83 mg, 0.23 mmol). ¹H NMR (400 MHz, Methanol-d4) δ 7.44 (m, 4H), 7.05 (m, 4H), 4.63 (m, J=6.2 Hz, 1H), 3.78 (m, 1H), 3.15 (m, 1H), 2.87 (d, J=13.2 Hz, 1H), 1.70 (m, 3H), 1.18 (m, 2H), 1.02 (s, 3H).

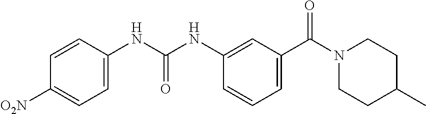 [SMILES: CC1CCN(C(=O)c2cccc(NC(=O)Nc3ccc([N+](=O)[O-])cc3)c2)CC1]

1-(3-(4-Methylpiperidine-1-carbonyl)phenyl)-3-(4-nitrophenyl)urea (12). Using the general procedures outlined above, 12 was synthesized as a yellow solid in 39% yield (68 mg, 0.18 mmol). ¹H NMR (400 MHz, Methanol-d4) δ 8.23 (d, J=9.24 Hz, 2H), 7.70 (d, J=9.24 Hz, 2H), 7.63 (s, 1H), 7.52 (d, J=8.16 Hz, 1H), 7.42 (t, J=7.60, 8.00, Hz, 1H), 7.08 (d, J=7.56 Hz, 1H), 4.62 (d, J=12.64 Hz, 1H), 3.77 (d, J=11.32 Hz, 1H), 3.15 (t, J=10.76, 12.44, Hz, 1H), 2.88 (t, J=11.68, 11.16, Hz, 1H), 1.72 (m, 3H), 1.22 (m, 2H), 1.02 (d, J=6.40 Hz, 3H).

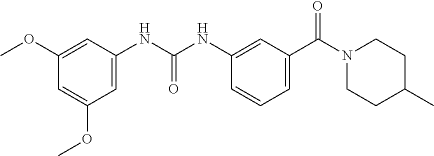

1-(3,5-Dimethoxyphenyl)-3-(3-(4-methylpiperidine-1-carbonyl)phenyl)urea (13). Using the general procedures outlined above, 13 was synthesized as a yellow solid in 36% yield (65 mg, 0.16 mmol). ¹H NMR (400 MHz, Methanol-d4) δ 7.99 (d, J=3.7 Hz, 1H), 7.55 (m, 2H), 7.49 (d, J=9.5 Hz, 1H), 7.34 (m, 2H), 6.60 (d, J=9.4 Hz, 2H), 4.61 (m, 1H), 3.82 (s, 6H), 3.49 (m, 1H), 3.10 (m, 1H), 2.77 (m, 1H), 1.63 (m, 3H), 1.09 (m, 2H), 0.096 (s, 3H).

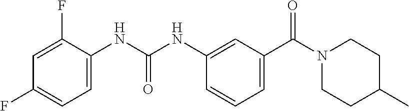 [SMILES: CC1CCN(C(=O)c2cccc(NC(=O)Nc3ccc(F)cc3F)c2)CC1]

1-(2,4-Difluorophenyl)-3-(3-(4-methylpiperidine-1-carbonyl)phenyl)urea (14). Using the general procedures outlined above, 14 was synthesized as a yellow solid in 62% yield (106 mg, 0.28 mmol). ¹H NMR (400 MHz, Methanol-d4) δ 8.12 (m, 2H), 7.89 (s, 1H), 7.70 (d, J=8.5 Hz, 1H), 7.54 (t, J=9.0 Hz, 8.3 1H), 7.46 (m, 1H), 7.19 (d, J=4.3 Hz, 1H), 4.63 (d, J=4.6 Hz, 1H), 3.49 (d, J=5.8 Hz, 1H), 3.16 (m, 1H), 2.87 (m, 1H), 1.80 (m, 3H), 1.22 (m, 2H), 1.02 (s, 3H).

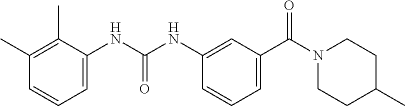

1-(2,3-Dimethylphenyl)-3-(3-(4-methylpiperidine-1-carbonyl)phenyl)urea (15). Using the general procedures outlined above, 15 was synthesized as a yellow solid in 41% yield (69 mg, 0.19 mmol). ¹H NMR (400 MHz, Methanol-d4) δ 7.59 (s, 1H), 7.47 (d, J=10 Hz, 1H), 7.36 (m, 2H), 7.04 (t, J=9.5, 9.3 Hz, 1H), 7.04 (t, J=9.8, 8.4 Hz, 2H), 4.61 (d, J=15.2 Hz, 1H), 3.47 (d, J=16.1 Hz, 1H), 3.14 (t, J=16.5, 15.1 Hz, 1H), 2.85 (t, J=17, 15.7 Hz, 1H), 2.33 (s, 3H), 2.23 (s, 3H), 1.70 (m, 3H), 1.18 (m, 2H), 1.01 (s, 3H).

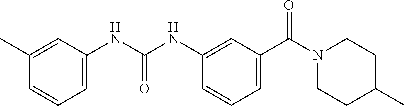

1-(3-(4-Methylpiperidine-1-carbonyl)phenyl)-3-(m-tolyl)urea (16). Using the general procedures outlined above, 16 was synthesized as a white solid in 49% yield (79 mg, 0.22 mmol). ¹H NMR (400 MHz, Methanol-d4) δ 7.85 (s, 1H), 7.48 (d, J=9 Hz, 1H), 7.39 (t, J=9.1 Hz, 9.9 1H), 7.27 (s, 1H), 7.21 (m, 1H), 7.04 (d, J=9.5 Hz, 2H), 4.73 (dd, J=8.3, 8.3 Hz, 1H), 4.62 (d, J=15.6 Hz, 1H), 3.77 (d, J=16.6 Hz, 1H), 3.14 (t, J=14.9, 16.1 Hz, 1H), 2.86 (t, J=14.2, 15 Hz, 1H), 1.73 (m, 3H), 1.21 (m, 2H), 1.01 (s, 3H).

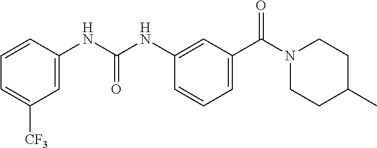 [SMILES: CC1CCN(C(=O)c2cccc(NC(=O)Nc3cccc(C(F)(F)F)c3)c2)CC1]

1-(3-(4-Methylpiperidine-1-carbonyl)phenyl)-3-(3-(trifluoromethyl)phenyl)urea (17). Using the general procedures outlined above, 17 was synthesized as a yellow solid in 64% yield (179 mg, 0.44 mmol). ¹H NMR (400 MHz, CDCl3) δ 7.66 (d, J=8.15 Hz, 2H), 7.57 (t, J=10.35 Hz, 1H), 7.37 (t, J=10.0, 10.5 Hz, 3H), 7.23 (d, J=9.9 Hz, 2H), 7.09 (s, 1H), 6.93 (d, J=9.53 Hz, 1H), 4.73 (d, J=16.6 Hz, 1H), 3.78 (d, J=16.4 Hz, 1H), 3.05 (t, J=15.0, 15.5 Hz, 1H), 2.88 (t, J=13.6, 13.3 Hz, 1H), 1.86 (d, J=16.2 Hz, 2H), 1.69 (m, 1H), 1.19 (m, 2H), 1.00 (s, 3H).

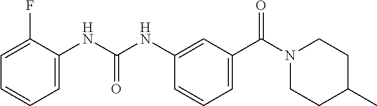 [SMILES: CC1CCN(C(=O)c2cccc(NC(=O)Nc3ccccc3F)c2)CC1]

1-(2-Fluorophenyl)-3-(3-(4-methylpiperidine-1-carbonyl)phenyl)urea (18). Using the general procedures outlined above, 18 was synthesized as a beige solid in 56% yield (91 mg, 0.26 mmol). ¹H NMR (400 MHz, DMSO-d6) δ 8.11 (m, 1H), 7.48 (s, 1H), 7.32 (m, 1H), 7.17 (m, 2H), 7.08 (t, J=9.5, 9.7 Hz, 1H), 6.95 (m, 2H), 4.35 (m, 1H), 2.93 (m, 1H), 2.67 (m, 1H), 1.55 (m, 3H), 0.99 (m, 2H), 0.86 (s, 3H).

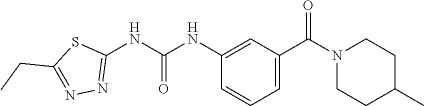 [SMILES: CCc1nnc(NC(=O)Nc2cccc(C(=O)N3CCC(C)CC3)c2)s1]

1-(5-Ethyl-1,3,4-thiadiazol-2-yl)-3-(3-(4-methylpiperidine-1-carbonyl)phenyl)urea (19). Compound 19 was purchased from Aronis.

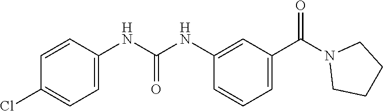 [SMILES: O=C(Nc1ccc(Cl)cc1)Nc1cccc(C(=O)N2CCCC2)c1]

1-(4-Chlorophenyl)-3-(3-(pyrrolidine-1-carbonyl)phenyl)urea (20). Using the general procedures outlined above, 20 was synthesized as a yellow solid in 39% yield (71 mg, 0.21 mmol). ¹H NMR (400 MHz, Methanol-d4) δ 7.44 (m, 4H), 7.39 (t, J=9.6, 10.1 Hz, 1H), 7.30 (d, J=10.9 Hz, 2H), 7.18 (d, J=9.4 Hz, 1H), 3.61 (t, J=8.6, 8.7 Hz, 2H), 3.51 (t, J 8.4, 8 Hz, 2H), 2.01 (q, J=8.4, 8.8 Hz, 2H), 1.93 (q, J=8.1, 8.3 Hz, 2H).

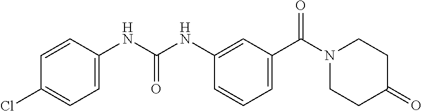 [SMILES: O=C1CCN(C(=O)c2cccc(NC(=O)Nc3ccc(Cl)cc3)c2)CC1]

1-(4-Chlorophenyl)-3-(3-(4-oxopiperidine-1-carbonyl)phenyl)urea (21). Using the general procedures outlined above, 21 was synthesized as a white solid in 12% yield (21 mg, 0.056 mmol). ¹H NMR (400 MHz, Methanol-d4) δ 7.69 (s, 1H), 7.46 (d, J=8.5 Hz, 1H), 7.33 (t, J=8.6, 9.5 Hz, 1H), 7.17 (d, J=9.1 Hz, 3H), 7.10 (dd, J=9.6, 5.7 Hz, 2H), 1.98 (m, 2H), 1.83 (m, 2H), 1.26 (m, 4H).

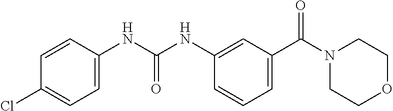 [SMILES: O=C(Nc1ccc(Cl)cc1)Nc1cccc(C(=O)N2CCOCC2)c1]

1-(4-Chlorophenyl)-3-(3-(morpholine-4-carbonyl)phenyl)urea (22). Using the general procedures outlined above, 22 was synthesized as a yellow solid in 13% yield (67 mg, 0.19 mmol). ¹H NMR (400 MHz, Methanol-d4) δ 7.64 (s, 1H), 7.45 (m, 4H), 7.29 (m, 2H), 7.10 (d, J=8.4 Hz, 1H), 3.77 (m, 4H), 3.67 (m, 2H), 3.51 (s, 2H).

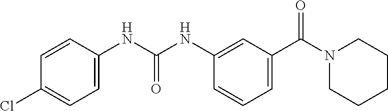 [SMILES: O=C(Nc1ccc(Cl)cc1)Nc1cccc(C(=O)N2CCCCC2)c1]

1-(4-Chlorophenyl)-3-(3-(piperidine-1-carbonyl)phenyl)urea (23). Using the general procedures outlined above, 23 was synthesized as a white solid in 11% yield (19 mg, 0.053 mmol). ¹H NMR (400 MHz, Methanol-d4) δ 7.61 (d, J=8.9 Hz, 1H), 7.46 (d, J=7.6 Hz, 1H), 7.37 (t, J=9.9, 7.3 Hz, 1H), 7.29 (d, J=8.5 Hz, 2H), 7.16 (t, J=9.6, 9.7 Hz, 1H), 7.02 (t, J=9.8, 9.6 Hz, 1H), 6.85 (d, J=8.4 Hz, 1H), 1.99 (m, 2H), 1.69 (m, 2H), 1.42 (m, 2H), 1.25 (m, 4H).

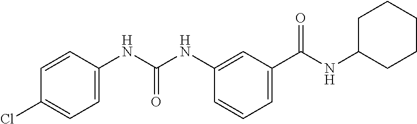 [SMILES: O=C(Nc1ccc(Cl)cc1)Nc1cccc(C(=O)NC2CCCCC2)c1]

3-(3-(4-Chlorophenyl)ureido)-N-cyclohexylbenzamide (24). Using the general procedures outlined above, 24 was synthesized as a white solid in 69% yield (61 mg, 0.16 mmol). ¹H NMR (400 MHz, Methanol-d4) δ 7.84 (s, 1H), 7.60 (d, J=10 Hz, 2H), 7.47 (d, J=8.6 Hz, 2H), 7.39 (t, J=9.9, 9.8 Hz, 1H), 7.30 (d, J=8.5 Hz, 1H), 7.16 (t, J=9.6, 9.7 Hz, 1H), 7.08 (t, J=9.8, 9.6 Hz, 1H), 6.86 (d, J=8.4 Hz, 1H), 3.85 (m, 1H), 1.99 (t, J=15, 14 Hz, 1H), 1.82 (m, 2H), 1.69 (m, 1H), 1.33 (m, 6H).

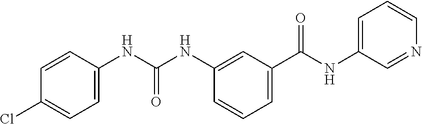 [SMILES: O=C(Nc1ccc(Cl)cc1)Nc1cccc(C(=O)Nc2cccnc2)c1]

3-(3-(4-Chlorophenyl)ureido)-N-(pyridin-3-yl)benzamide (25) Using the general procedures outlined above, 25 was synthesized as a white solid in 21% yield (37 mg, 0.10 mmol). ¹H NMR (400 MHz, Methanol-d4) δ 8.92 (s, 1H), 8.33 (d, J=4.68 Hz, 1H), 8.29 (d, J=2.92 Hz, 1H), 8.05 (s, 1H), 7.64 (m, 2H), 7.45 (m, 4H), 7.30 (t, J=7.00, 6.96 Hz, 1H).

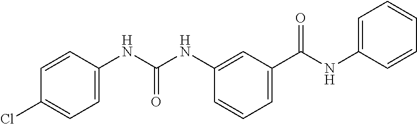 [SMILES: O=C(Nc1ccc(Cl)cc1)Nc1cccc(C(=O)Nc2ccccc2)c1]

3-(3-(4-Chlorophenyl)ureido)-N-phenylbenzamide (26) Using the general procedures outlined above, 26 was synthesized as a white solid in 39% yield (67 mg, 0.18 mmol). ¹H NMR (400 MHz, DMSO-d6) δ 8.93 (d, J=8.1 Hz, 2H), 7.69 (d, J=8.1 Hz, 2H), 7.57 (d, J=9.8 Hz, 1H), 7.51 (m, 2H), 7.44 (t, J=9.9, 9.8 Hz, 1H), 7.35 (m, 4H), 7.10 (t, J=9.3, 9.3 Hz, 1H).

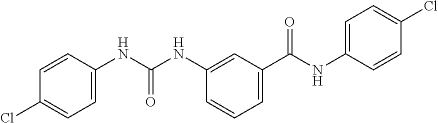 [SMILES: O=C(Nc1ccc(Cl)cc1)Nc1cccc(C(=O)Nc2ccc(Cl)cc2)c1]

N-(4-Chlorophenyl)-3-(3-(4-chlorophenyl)ureido)benzamide (27). Using the general procedures outlined above, 27 was synthesized as a yellow solid in 53% yield (86 mg, 0.21 mmol). H NMR (400 MHz, Methanol-d4) δ 8.85 (d, J=8.3 Hz, 1H), 7.81 (d J=10.5 Hz, 1H), 7.68 (d, J=9.5 Hz, 1H), 7.48 (m 6H), 7.09 (m, 3H).

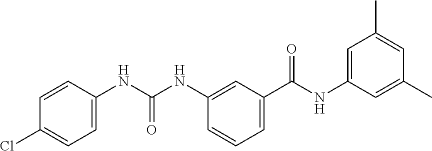 [SMILES: Cc1cc(C)cc(NC(=O)c2cccc(NC(=O)Nc3ccc(Cl)cc3)c2)c1]

3-(3-(4-Chlorophenyl)ureido)-N-(3,5-dimethylphenyl)benzamide (28). Using the general procedures outlined above, 28 was synthesized as a white solid in 41% yield (67 mg, 0.17 mmol). ¹H NMR (400 MHz, DMSO-d6) δ 8.93 (s, 1H), 8.89 (s, 1H), 7.94 (s, 1H), 7.57 (d, J=10 Hz, 1H), 7.52 (m, 4H), 7.34 (m, 2H), 7.08 (s, 1H), 3.74 (s, 6H).

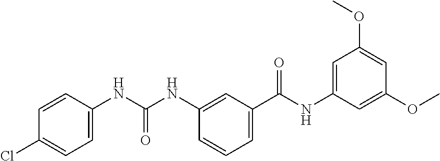 [SMILES: COc1cc(NC(=O)c2cccc(NC(=O)Nc3ccc(Cl)cc3)c2)cc(OC)c1]

3-(3-(4-Chlorophenyl)ureido)-N-(3,5-dimethoxyphenyl)benzamide (29). Using the general procedures outlined above, 29 was synthesized as a white solid in 16% yield (39 mg, 0.099 mmol). ¹H NMR (400 MHz, DMSO-d6) δ 8.93 (s, 1H), 8.89 (s, 1H), 7.66 (d, J=10.05 Hz, 1H), 7.49 (m, 3H), 7.35 (d, J=11.05 Hz, 2H), 7.08 (s, 2H), 6.26 (m, 1H), 3.35 (s, 6H).

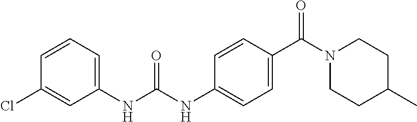 [SMILES: CC1CCN(C(=O)c2ccc(NC(=O)Nc3cccc(Cl)c3)cc2)CC1]

1-(4-(4-Methylpiperidine-1-carbonyl)phenyl)-3-(m-tolyl)urea (30). Using the general procedures outlined above, 30 was synthesized as a white solid in 13% yield (67 mg, 0.18 mmol). ¹H NMR (400 MHz, Methanol-d4) δ 7.55 (d, J=10.9 Hz, 2H), 7.47 (d, J=11.2 Hz, 2H), 7.37 (d, J=10.8 Hz, 2H), 7.30 (d, J=11.15 Hz, 2H), 4.58 (m, 1H), 3.83 (m, 1H), 3.15 (m, 1H), 2.86 (m, 1H), 1.71 (m, 3H), 1.19 (m, 2H), 1.01 (m, 2H).

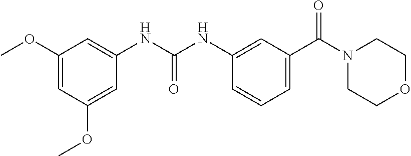 [SMILES: COc1cc(NC(=O)Nc2cccc(C(=O)N3CCOCC3)c2)cc(OC)c1]

1-(3,5-Dimethoxyphenyl)-3-(3-(morpholine-4-carbonyl)phenyl)urea (31). Using the general procedures outlined above, 31 was synthesized as a white solid in 28% yield (67 mg, 0.17 mmol). ¹H NMR (400 MHz, CDCl3) δ 7.56 (m, 2H), 7.22 (m, 1H), 7.12 (s, 1H), 6.90 (d, J=Hz, 9.5 1H), 6.56 (s, 1H), 6.13 (s, 1H), 3.77 (m, 4H), 3.71 (s, 6H), 3.57 (m, 2H), 3.42 (m, 2H).

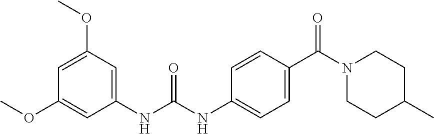

1-(3,5-Dimethoxyphenyl)-3-(4-(4-methylpiperidine-1-carbonyl)phenyl)urea (32). Using the general procedures outlined above, 32 was synthesized as a yellow solid in 54% yield (98 mg, 0.25 mmol). ¹H NMR (400 MHz, CDCl3) δ 7.41 (d, J=10.70 Hz, 2H), 7.23 (d, J=10.65. Hz 2H), 6.61 (m, 3H), 6.08 (t, J=2.70, 2.70 Hz, 1H), 3.65 (s, 6H), 1.56 (m, 4H), 1.13 (m, 2H), 1.00 (m, 3H), 0.86 (s, 3H).

### Biological Evaluation of Compounds

### Compound Overview

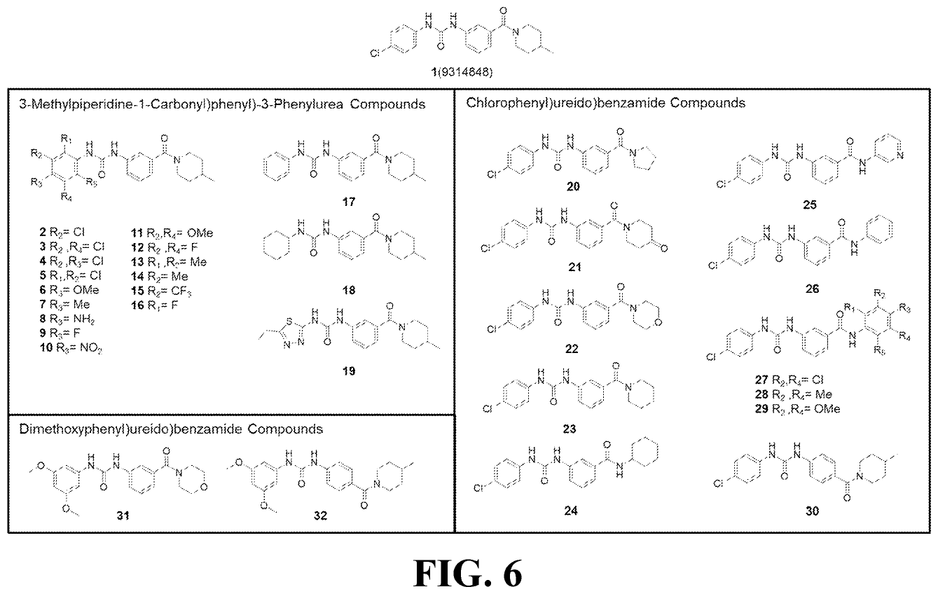

The chemical scaffold of compound 9314848 served as the scaffold for compounds that were synthesized. Using a synthetic approach, compounds 2-19 were assembled to study substitution patterns on the benzene ring of 9314848 and additional aromatic and aliphatic groups attached directly to the urea (FIG. 6). Henceforth, the 9314848 scaffold will be referenced as compound 1 (9314848). Several derivatives were designed from compound 1 (9314848) that possess chloro substituents, including 2, 3, 4, and 5, to determine the effect that the number and placement of chlorine had on activity. Methoxy (compound 6), methyl (compound 7), and amino (compound 8) groups were introduced at the 4-position to compare effects of electron donating groups versus the electron withdrawing chlorine. A methyl group was also selected because it is the steric isostere of chlorine. Additional electron withdrawing groups were also selected for the 4-possition, including a fluoro group (compound 9) and a nitro group (compound 10). Derivatives containing electron donating and electron withdrawing groups at various other positions were also synthesized. For example, compound 11, which possesses methoxy groups at the 3- and 5-positions, and compound 12, which possesses fluoro groups at these same positions. These compounds have been designated as (3-methylpiperidine-1-carbonyl)phenyl)-3-phenylurea compounds

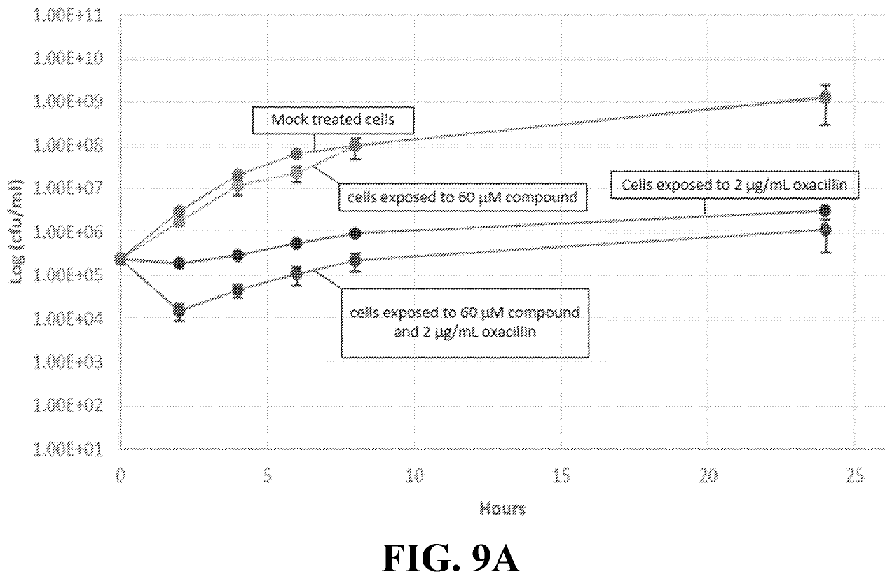

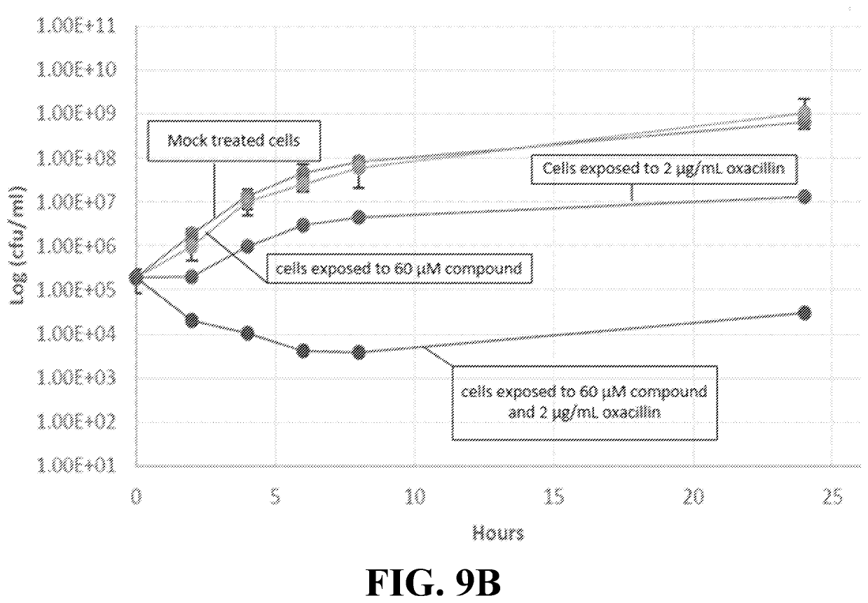

The library was expanded by synthesizing compounds 20-30 (FIG. 6). In this group of compounds, identity of the group attached to the benzoamide was studied. Compound 20 was synthesized with a pyrrolidine group to determine the effect that a smaller ring has on potency. Compounds 21 and 22 incorporate an oxygen to the aliphatic ring through a piperidone and morpholine group, respectively. Compound 23 was synthesized with a piperidine in place of the methyl piperidine and compound 24 contains an aliphatic cyclohexane. Like compound 24, several other derivatives that were synthesized included a secondary amide head instead of the tertiary amide found in compound 1 (9314848). Further synthesis involved synthesizing compound 26 to possess a phenyl group to study the effects of aromaticity on the amide head and to compare with derivatives that contain secondary substituted aryl amides. Additionally, compound 27 was synthesized to contain a 4-chlorophenyl group similar to the para-chlorophenyl substituent attached to the urea. Compounds 28 and 29 were synthesized to study methyl and methoxy substitution at the 3- and 5-positions, respectively. Compound 29 was synthesized to study the effect of the 3,5-dimethoxy phenyl group that also exists in compound 11 (FIG. 6). Compound 30 was also synthesized to determine the impact that moving the amide to the para position had upon potency. Compound 11 and compound 22 were combined to make compound 31. Compound 31 possesses the 3,5-dimethoxy phenyl employed in compound 11 and the morpholine group from compound 22, (FIG. 9). In the text, compounds 20-30 are referred generally as 3-(3-(4-chlorophenyl)ureido)benzoamides and compounds 31-32 are referred generally as 3-(3-(3,5-dimethoxyphenyl)ureido)benzamides.

### Binding Interaction of Substituted (3-Methylpiperidine-1-Carbonyl)phenyl)-3-Phenylurea Compounds to PBP4 and PBP2A

To assess activity, binding to recombinant PBP4 and PBP2a was determined via a Bocillin™ fluorescence assay (Table 3). Bocillin™-FL is a fluorescent penicillin used as a labeling reagent for the detection of PBPs. This assay allows for the measurement of the ability of derivatives to displace Bocillin™-FL and bind to either PBP4 or PBP2A. The data is expressed as percentage inhibition of Bocillin™-FL binding. All compounds were assayed at 50 μM. The binding of 1 (9314848) was previously determined to be 69.2%±14.6 towards PBP4 and 24% 2.1 towards PBP2a.

TABLE 3
Percent bocillin ™ displacement of compounds.
Compounds were used at 50 μM and PBPs at
1 μM in bocillin ™ assays.

Bocillin ™ Displacement (%)

| Compound | PBP4 | PBP2a |
|---|---|---|
| 1 | 69.2 ± 14.6 | 24 ± 2.1 |
| (9314848) | | |
| 2 | 85.8 ± 8.6 | 26 ± 10.7 |
| 3 | <1.0 | <1.0 |
| 4 | 83.9 ± 8.8 | <1.0 |
| 5 | 68.5 ± 5.7 | <1.0 |
| 6 | 22.8 ± 12.8 | <1.0 |
| 7 | 65.1 ± 3.1 | <1.0 |
| 8 | 63.9 ± 7.8 | 21 ± 3.6 |
| 9 | 50 ± 5.6 | 28 ± 6.7 |
| 10 | 81.4 ± 9.2 | 15 ± 2.6 |
| 11 | 74.9 ± 21.1 | 35 ± 13.6 |
| 12 | 60.5 ± 5.1 | 25 ± 3.1 |
| 13 | 66.9 ± 8.3 | 15 ± 10.4 |
| 14 | 63.5 ± 3.4 | <1.0 |
| 15 | 63.5 ± 3.4 | <1.0 |
| 16 | 65.9 ± 9.3 | <1.0 |
| 17 | 43.6 ± 5.9 | <1.0 |
| 18 | 31.8 ± 3.5 | 29 ± 13.6 |
| 19 | 19.4 ± 3.1 | 24.3 ± 26.4 |

From this binding screen, 13 of the 18 compounds showed greater displacement of Bocillin™-FL labeling of PBP4 in comparison to 1 (9314848), while the other five were not as active. Of the analogs that maintained a phenyl-based tail, switching from a para-chloro substituent in compound 1 (9314848) to a 3-,5-dichloro arrangement (compound 3), a para-methoxy (compound 6), or removal of the chlorine (compound 17) displayed greater binding capabilities by the reduction in Bocillin™-FL labeling, while all other derivatives exhibited similar activity to compound 1 (9314848). Replacement of the phenyl tail with a cyclohexyl substituent or an alternative heterocycle (a 2-ethyl-1,3,4-thiadiazole) attenuated activity. As a preliminary gauge of specificity, each compound was evaluated at a concentration of 50 μM for inhibition of Bocillin™-FL labeling of PBP2a (Table 3). For reference, compound 1 (9314848) showed lower affinity for PBP2a, inhibiting 24±2.1% Bocillin™ binding at 50 mM. With the exception of compound 19, all tail analogs followed a similar trend and showed a reduced ability to inhibit Bocillin™-FL labelling of PBP2a. Interestingly, compounds 3, 4, 5, 6, 7, 14, 15, 16 and 17 showed no binding to PBP2a, and compound 3 showed no binding activity towards PBP2a.

### Toxicity of Substituted (3-Methylpiperidine-1-Carbonyl)phenyl)-3-Phenylurea Compounds in HEPG2 Cells

To determine the potential toxicity in mammalian cells, each analog was evaluated at 100 μM in a cell viability assay using HepG2 cells. Compounds resulting in cell viability above 70% were considered non-cytotoxic. Previously, treatment with compound 1 (9314848) was shown to produce 84.2±15.3% cell viability. The majority of compounds exhibited a low level of toxicity similar to compound 1 (9314848). Compounds 12 (58.9±9.2%) and 14 (54.5±4.9%) displayed the greatest degree toxicity (Table 4).

TABLE 4
Cell viability of substituted 3-methylpiperidine-1-carbonyl)phenyl)-
3-phenylurea compounds at 100 μM. HEPG2 cells were used at a
concentration of approximately 2.5 × 10⁵ cells/mL.

| | Cell Viability |
|---|---|
| Compound | (%) |
| 1 | 84.2 ± 15.3 |
| (9314848) | |
| 2 | 80.8 ± 22.2 |
| 3 | 122.2 ± 7.1 |
| 4 | 75.4 ± 3.2 |
| 5 | 93.1 ± 22.1 |
| 6 | 91.2 ± 7.7 |
| 7 | 96.2 ± 13.5 |
| 8 | 85.7 ± 11.1 |
| 9 | 96.1 ± 42.7 |
| 10 | 75.7 ± 3.6 |
| 11 | 82.9 ± 24.9 |
| 12 | 58.9 ± 9.2 |
| 13 | 67.6 ± 15.5 |
| 14 | 54.5 ± 4.9 |
| 15 | 109.8 ± 18.6 |
| 16 | 112.5 ± 18.1 |
| 17 | 74.9 ± 9.4 |
| 18 | 93.9 ± 11.7 |
| 19 | 100.7 ± 5.2 |

Inhibition of S. aureus Migration

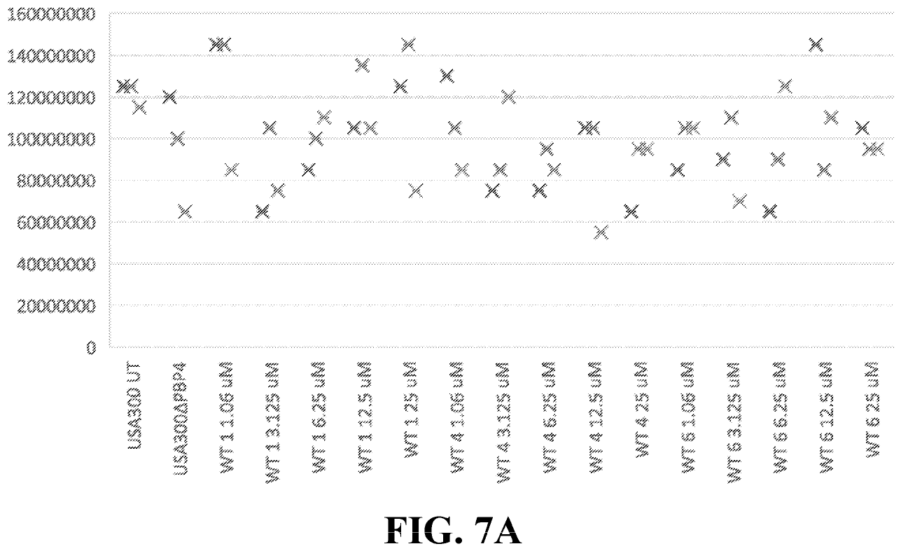

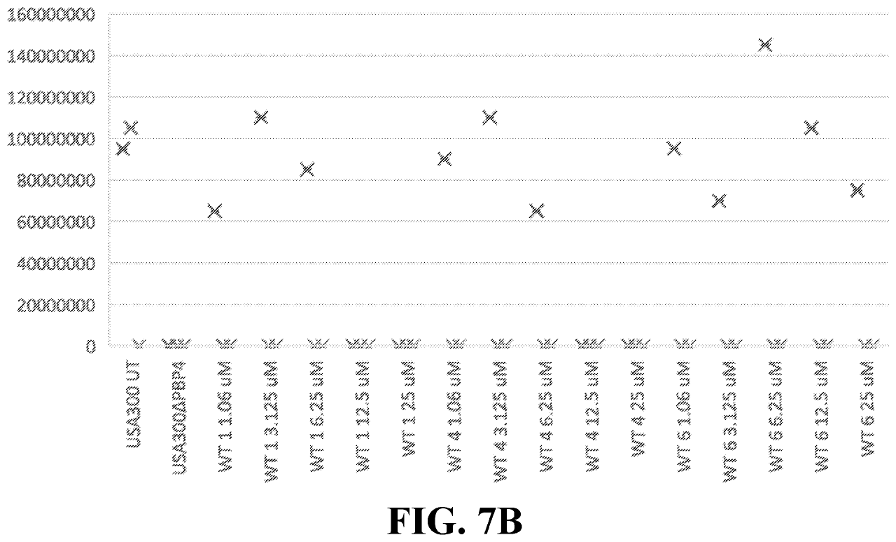

Finally, the ability of select derivatives to inhibit the ability of S. aureus to migrate through nanoporous membranes was evaluated using a μSiM-CA device that mimics the orifices of osteocyte lacuna-canalicular networks (FIGS. 7A-7B). This μSIM-CA system includes a top and a bottom chamber separated by a 400 nm thick silicon nitride membrane (containing 500 nm pores), which allows for quantification of bacteria that migrate from the top (FIG. 7A) well to the bottom (FIG. 7B) well. Since migration in this assay is dependent upon PBP4 function, compounds that inhibit PBP4 activity in culture should inhibit the ability of S. aureus to migrate through these channels. Lead compound 1 (9314848) prevented USA300 from crossing the membrane in a dose-dependent matter, showing full inhibition at 12.5 μM. Compound 4, which displayed equivalent binding activity compared to compound 1 (9314848), also showed dose-dependent inhibition of migration with full inhibition at 12.5 mM. Compound 6, which was less active in binding PBP4 compared to compound 1 (9314848) and compound 4, was shown to have a weaker ability to prevent USA300 from crossing the membrane and showed no difference between treatment at the lowest and highest concentrations tested.

### Potentiation of Oxacillin by Substituted (3-Methylpiperidine-1-Carbonyl)phenyl)-3-Phenylurea Compounds Against MRSA Strains

Compounds were evaluated at 60 mM with oxacillin as the representative β-lactam against three different MRSA strains (AH-1263, AH-2204, and ATCC BAA-1556) (Table 5). Compound 1 (9314848) reduced the oxacillin MIC 16-fold, 32-fold, and 16-fold against test strains AH-1263, AH-2204, and ATCC BAA-1556, respectively. Compound 2, with a chloro in the 3-position, was equipotent to compound 1 (9314848) against strain AH-1263 although less potent towards AH-2204 and ATCC BAA-1556. Compound 3, in which chloro substituents are in the 3- and 5-positions exhibited increased potency, dropping the MIC of oxacillin by 64-fold, 256-fold, and 128-fold, an increase of four-fold, eight-fold, and eight-fold compared to compound 1 (9314848) against AH-1263, AH-2204, and ATCC BAA-1556, respectively. Likewise, compound 4, which possesses chloro substituents in the 3- and 4-positions was more potent than 1 (9314848), reducing the MIC of oxacillin by 32-fold, 128-fold, and 64-fold towards AH-1263 and ATCC BAA-1556, and AH-2204, respectively. It is accepted in the field that when the MIC change is less than a four-fold difference that the compounds are equipotent. The fold difference between compound 1 (9314848) and 4 at AH-1263, ATCC-1556, and AH-2204 are two-fold, four-fold, and four-fold, respectively. Since only two-fold difference between 4 and 1 (9314848) exists for AH-1263, these compounds are equipotent at AH-1263. Compound 3 was also two-fold more active than compound 4 against all three MRSA strains, indicating similar potency. Compound 5, with chloro substituents in the 2- and 3-positions, was less active than 1 (9314848) against all MRSA strains.

Compounds 6, 7, 8, 9 and 10 have a functional group at only the 4-position, similar to parent 1 (9314848), and were less potent or equally as potent compared to compound 1 (9314848). Between compounds containing methoxy (compound 11) and fluoro (compound 12) substitutes in the 3- and 5-positions, it was observed that compound 11 was more potent than compound 12 by four-fold against AH-1263 and AH-2204, and equipotent against ATCC BAA-1556. Compounds 13 and 14 both have methyl groups in the 2-, 3- and 2-positions, respectively. Compound 13 was more potent than compound 14 by at least four-fold against all strains. Compound 13 was also more active than compound 5, which possesses a 2,3-dichloro substitution pattern, showing that the methyl groups increased potency compared to chlorine at these positions (Table 5).

TABLE 5
Potentiation of oxacillin by substituted 3-methylpiperidine-
1-carbonyl)phenyl)-3-phenylurea compounds at 60 mM against
MRSA strains AH-1263, AH-2204, and ATCC BAA-1556.
Oxacillin MIC (μg/mL) (fold reduction)

| Compounds | AH-1263 | AH-2204 | BAA-1556 |
|---|---|---|---|
| — | 16 | 32 | 32 |
| 1 | 1 (16) | 1 (32) | 2 (16) |
| (9314848) | | | |
| 2 | 1 (16) | 8 (4) | 8 (4) |
| 3 | 0.25 (64) | 0.125 (256) | 0.25 (128) |
| 4 | 0.5 (32) | 0.25 (128) | 0.5 (64) |
| 5 | 2 (8) | 1 (32) | 4 (8) |
| 6 | 4 (4) | 1 (32) | 8 (4) |
| 7 | 1 (16) | 0.5 (64) | 4 (8) |
| 8 | 4 (4) | 2 (16) | 8 (4) |
| 9 | 2(8) | 1 (32) | 2 (16) |
| 10 | 0.5 (32) | 0.5 (64) | 2 (16) |
| 11 | 0.125 (128) | 0.125 (256) | 0.25 (128) |
| 12 | 0.5 (32) | 0.5 (64) | 0.25 (128) |
| 13 | 0.25 (64) | 0.25 (128) | 0.25 (128) |
| 14 | 2 (8) | 0.5 (64) | 1 (32) |
| 15 | 1 (16) | 0.5 (64) | 0.5 (64) |
| 16 | 0.25 (64) | 0.25 (128) | 0.25 (128) |
| 17 | 1 (16) | 0.5 (64) | 0.5 (64) |
| 18 | 8 (2) | 2 (16) | 8 (4) |
| 19 | 0.5 (32) | 2 (16) | 4 (8) |

Comparison of compounds 11, 13 and 14, which all possess electron donating groups, indicated that compounds 11 and 13 were equipotent and both more active than 14 against at least two strains. Compounds 14 (—CH₃) and 15 (—CF₃) are both substituted in the 3-position, and no significant difference in potency was observed. Comparing compound 2, which possesses a 3-chloro moiety, to compounds 14 and 15 revealed that both were more potent than compound 2 against strains AH-2204, and ATCC BAA-1556, although less potent or equally as potent against AH-1263.

Compound 17, which contains a phenyl group, was synthesized to compare the impact that the lack of substitution had upon activity. When the chlorine was removed, potency was improved towards strain ATCC BAA-1556 by four-fold compared to 1 (9314848). A cyclohexyl group was also employed instead of an aryl group (compound 18) and was less potent than the parent compound. Compound 19, which possesses a 2-ethyl-1,3,4-thiadiazole moiety, was more potent compared to 1 (9314848) against any strain. Compounds from this first group that decreased the MIC of oxacillin more than compound 1 against all MRSA strains were compounds 3, 4, 11, 12, 13 and 16. The most potent compound was 11, lowering the MIC by 128-fold, 256-fold, and 128-fold against AH-1263, AH-2204, and ATCC BAA-1556, respectively (Table 5). These six compounds all contain a functional group in either the 2 or 3 positions.

### Potentiation of Oxacillin by Substituted 3-(3-(4-Chlorophenyl)ureido)benzamide Compounds Against MRSA Strains

Of the derivatives 20-29, only compound 22 displayed increased potency towards more than one MRSA strain compared with compound 1 (9314848), dropping the MIC of oxacillin by 32-fold, 128-fold, and 64-fold against AH-1263, AH-2204, and ATCC BAA-1556, respectively (Table 6).

TABLE 6
Potentiation of oxacillin by substituted 3-(3-(4-
chlorophenyl)ureido)benzamide compounds at 60 mM against
MRSA strains AH-1263, AH-2204, and ATCC BAA-1556.
Oxacillin MIC (μg/mL) (fold reduction)

| | | | ATCC |
|---|---|---|---|
| Compounds | AH-1263 | AH-2204 | BAA-1556 |
| — | 16 | 32 | 32 |
| 1 | 1 (16) | 1 (32) | 2 (16) |
| (9314848) | | | |
| 20 | 1 (16) | 1 (32) | 0.5 (64) |
| 21 | 32 (—) | 4 (8) | 16 (2) |
| 22 | 0.5 (32) | 0.25 (128) | 0.5 (64) |
| 23 | 16 (—) | 2 (16) | 16 (2) |
| 24 | 8 (2) | 2 (16) | 8 (4) |
| 25 | 64 (—) | 4 (8) | 64 (—) |
| 26 | 2 (8) | 1 (32) | 8 (4) |
| 27 | 1 (16) | 0.5 (64) | 4 (8) |
| 28 | 8 (2) | 2 (16) | 16 (2) |
| 29 | 64 (—) | 64 (—) | 32 (—) |
| 30 | 1 (16) | 0.25 (128) | 0.5 (64) |

This represents an increase in potency compared to compound 1 (9314848) by two-fold, four-fold, and four-fold, respectively. Compound 22 has a morpholine group in place of the methyl piperidine, which presumably increases the polarity and water solubility of the compound. Compound 20, which incorporates a pyrrolidine group at the head end of the compound, decreased the oxacillin MIC by four-fold against ATCC BAA-1556 compared to compound 1 (9314848). Aside from this activity, no other compounds decreased the MIC of oxacillin by ≥4-fold. Compound 30 was four-fold more potent than compound 1 (9314848) against strains AH-2204 and ATCC BAA-1556, and was equipotent towards AH-1263.

### Potentiation of Oxacillin by Substituted 3-(3-(3,5-Dimethoxyphenyl)ureido)benzamide Compounds Against MRSA Strains

Compound 32 was also synthesized as a hybrid of compounds 11 and 30, due to the similar activity that compound 30 displayed compared to compound 1 (9314848) (Table 6). Hybrid compound 31 lowered the oxacillin MIC by four-fold against AH-2204 and 32-fold against ATCC BAA-1556, although did not demonstrate activity towards strain AH-1263 (Table 7).

TABLE 7
Potentiation of oxacillin by substituted 3-(3-(3,5-
dimethoxyphenyl)ureido)benzamide compounds 30, 31, and 32 at
60 mM against MRSA strains AH-1263, AH-2204, ATCC BAA-1556.
Oxacillin MIC (μg/mL) (fold reduction)

| | | | ATCC |
|---|---|---|---|
| Compounds | AH-1263 | AH-2204 | BAA-1556 |
| — | 16 | 32 | 32 |
| 1 | 1 (16) | 1 (32) | 2 (16) |
| (9314848) | | | |
| 11 | 0.125 (128) | 0.125 (256) | 0.25 (128) |
| 22 | 0.5 (320 | 0.25 (128) | 0.5 (64) |
| 30 | 1 (16) | 0.25 (128) | 0.5 (64) |
| 31 | 32 (—) | 8 (4) | 1 (32) |
| 32 | 2 (8) | 0.5 (64) | 0.5 (64) |

Compound 31 was less active than compounds 11 and 22 against all MRSA strains. Compared to compound 1 (9314848), compound 31 was equipotent against ATCC BAA-1556 but was less active against AH-1263 and AH-2204. Compound 32 showed increased activity compared to compound 1 (9314848) against ATCC BAA-1556, lowering the MIC by an additional four-fold (Table 7). Compound 32 did not increase potency compared to compounds 11 and 30.

All compounds, with the exception of 29, lowered the MIC of oxacillin by at least four-fold against one or more strains of MRSA. Of these derivatives, 23 compounds lowered the oxacillin MIC by at least 32-fold, and eight compounds by at least 128-fold against one or more strains. Typically, derivatives in group 1 exhibited greater potency than those from group two. It is also interesting to note that although the most potent derivative, compound 11, possesses a 3,5-dimethoxybenzyl group at the urea tail, when this group was employed at the amide head in compound 29, the potentiation activity decreased dramatically. A similar relationship was observed between compounds 18 and 26 which both possesses phenyl groups in the respective positions. Compared to compound 26, compound 18, lowered the oxacillin MIC by a further 16-fold against ATCC BAA-1556, although was equal in potency towards AH-12163 and AH-2204. This trend was not observed between compounds 15 and 24 which possess cyclohexyl groups and were equally as potent across all MRSA strains.

### Potentiation of β-Lactams by Substituted Phenylureidobenzamide Compounds Against MRSA Strains

Nine derivatives were selected and evaluated for activity with a panel of ß-lactam antibiotics. Compounds 2 and 6 were chosen for their lower activity with regards to potentiating oxacillin to determine if these derivatives were more potent when coupled with different β-lactam antibiotics (Table 5). Compounds 4 and 10 were selected because of their equivalent binding affinity towards PBP4 when compared to 1 (9314848). Compounds 11, 13 and 16 were selected for their high potency when used with oxacillin. Compound 22 was chosen because it showed the greatest activity of the (3-methylpiperidine-1-carbonyl)phenyl)-3-phenylurea compounds (Table 6). Compound 29 was selected due to its complete lack of activity. Select compounds were first evaluated in tandem with three penicillin antibiotics: ampicillin, amoxicillin, and penicillin G (Tables 8A-8C). Ampicillin and amoxicillin were chosen because they are broad spectrum antibiotics, while penicillin G was selected because it is a narrow spectrum antibiotic like oxacillin.

TABLE 8A
Potentiation of amoxicillin by select compounds at
60 mM against AH-1262, AH-2204, and ATCC BAA-1556.

MIC (μg/mL) (fold reduction)
Amoxicillin

| | | | ATCC |
|---|---|---|---|
| Compound | AH-1263 | AH-2204 | BAA-1556 |
| — | 4 | 2 | 8 |
| 1 | 0.25(16) | 1(2) | 1(8) |
| (9314848) | | | |
| 2 | 1(4) | 0.5(4) | 1(8) |
| 4 | 4(—) | 1(2) | 64(0.12) |
| 6 | 8(0.5) | 1(2) | 4(2) |
| 10 | 8(0.5) | 2(—) | 8(—) |
| 11 | 2(2) | 4(0.5) | 8(—) |
| 13 | 1(4) | 0.5(4) | 0.5(16) |
| 16 | 2(2) | 0.5(4) | 2(4) |
| 22 | 8(0.5) | 1(2) | 4(2) |
| 29 | 16 (0.25) | 4(—) | 16(—) |

TABLE 8B
Potentiation of ampicillin by select compounds at
60 mM against AH-1262, AH-2204, and ATCC BAA-1556.

MIC (μg/mL) (fold reduction)
Ampicillin

| | | | ATCC |
|---|---|---|---|
| Compound | AH-1263 | AH-2204 | BAA-1556 |
| — | 16 | 2 | 16 |
| 1 | 1(16) | 1(2) | 1(16) |
| (9314848) | | | |
| 2 | 1(16) | 0.5(4) | 1(16) |
| 4 | 2(8) | 1(2) | 4(4) |
| 6 | 8(2) | 1(2) | 16(—) |
| 10 | 4(4) | 1(2) | 4(4) |
| 11 | 4(4) | 1(2) | 2(8) |
| 13 | 8(2) | 1(2) | 16(—) |
| 16 | 8(2) | 0.5(4) | 8(2) |
| 22 | 8(2) | 8(0.25) | 8(2) |
| 29 | 16(—) | 4(0.5) | 16(—) |

TABLE 8C
Potentiation of penicillin G by select compounds at
60 mM against AH-1262, AH-2204, and ATCC BAA-1556.

MIC (μg/mL) (fold reduction)
Penicillin G

| | | | ATCC |
|---|---|---|---|
| Compound | AH-1263 | AH-2204 | BAA-1556 |
| — | 16 | 32 | 32 |
| 1 | 0.062(256) | 0.125(256) | 0.015(256) |
| (9314848) | | | |
| 2 | 0.125(128) | 0.125 (256) | 0.125 (256) |
| 4 | 2(8) | 0.25(128) | 16(2) |
| 6 | 1(16) | 0.25(128) | 2(16) |
| 10 | 2(8) | 0.25(128) | 4(8) |
| 11 | 8(2) | 16(2) | 8(4) |
| 13 | 8(2) | 1(32) | 8(4) |
| 16 | 0.25(64) | 0.25(128) | 1(32) |
| 22 | 1(16) | 0.125(256) | 2(16) |
| 29 | 8(2) | 1(32) | 8(4) |

Of the eleven compounds evaluated here, five reduced the MIC of amoxicillin by four-fold against at least two MRSA strains (Table 8A). The most potent compounds were compounds 1 (9314848) and 13, which lowered the MIC by 16-fold against strains AH-1263 and ATCC BAA-1556 respectively. In combination with ampicillin, six compounds were able to lower the MIC by four-fold against at least one strain. Compounds 1 (9314848) and 2 were the most potent here, lowering the MIC by 16-fold against both AH-1263 and ATCC BAA-1556. Compounds potentiated the activity of penicillin G to the greatest degree with all compounds lowering the MIC at least fourfold against at least one MRSA strain. Compound 1 (9314848) was the most potent compound, lowering the MIC 2048-fold against ATCC BAA-1556, while compounds 2 and 22 were the most potent analogs, lowering the MIC 256-fold against AH-2204 and ATCC BAA-1556 (Table 8C). In contrast to the results observed with oxacillin, compound 11 was not the most potent derivative when used with any of these three β-lactams and was the least active compound when combined with penicillin G against AH-2204 (Table 8C). As expected, 29 was inactive, except when in combination with penicillin G against AH-2204 and ATCC BAA-1556 where it lowered the MIC by 32-fold, and four-fold, respectively (Table 8C).

Of the eleven compounds evaluated here, five reduced the MIC of amoxicillin by four-fold against at least two MRSA strains (Table 8A). The most potent compounds were compounds 1 (9314848) and 13, which lowered the MIC by 16-fold against strains AH-1263 and ATCC BAA-1556 respectively. In combination with ampicillin, six compounds were able to lower the MIC by four-fold against at least one strain (Table 8B).

### Potentiation of Cephalosporins by Substituted Phenylureidobenzamide Compounds Against MRSA Strains

Compounds were subsequently evaluated in tandem with three broad spectrum cephalosporins: cefoxitin (2nd generation), cefotaxime (3rd generation) and cephalothin (1^{st} generation) (Tables 9A-9C). Five of the ten compounds lowered the cefoxitin MIC by at least four-fold against at least one MRSA strain, while compound 13 was the most potent lowering the MIC by eight-fold against AH-1263. Five of the ten compounds reduced the cefotaxime MIC by at least four-fold, with the most potent derivatives, compounds 11 and 13, lowering the MIC by 16-fold against AH-1263. All compounds, with the exception of 29, reduced the MIC of cephalothin by at least four-fold against at least two MRSA strains. The most potent compound was compound 2, which reduced the MIC of cephalothin by 64-fold against AH-1263 (Table 9C). Compound 29 was inactive when used with cefoxitin, cefotaxime, or cephalothin.

TABLE 9A
Potentiation of cefoxitin by select compounds at 60 mM against
MRSA strains AH-1263, AH-2204, and ATCC BAA-1556.

Cefoxitin MIC (μg/mL) (fold reduction)

| | | | ATCC |
|---|---|---|---|
| Compound | AH-1263 | AH-2204 | BAA-1556 |
| — | 4 | 2 | 8 |
| 1 | 16(2) | 16(—) | 8(4) |
| (9314848) | | | |
| 2 | 8(4) | 4(4) | 8(4) |
| 4 | 16(2) | 8(2) | 64(0.5) |
| 6 | 16(2) | 8(2) | 32(—) |
| 10 | 32(—) | 16(—) | 32(—) |
| 11 | 32(—) | 32(0.5) | 32(—) |
| 13 | 4(8) | 4(4) | 16(2) |
| 16 | 8(4) | 8(2) | 16(2) |
| 22 | 8(4) | 8(2) | 16(2) |
| 29 | 64(0.5) | 32(0.5) | 64(0.5) |

TABLE 9B
Potentiation of cefotaxime by select compounds at 60 mM
against MRSA strains AH-1263, AH-2204, and ATCC BAA-1556.

Ceftoaxime MIC (μg/mL) (fold reduction)

| | | | ATCC |
|---|---|---|---|
| Compound | AH-1263 | AH-2204 | BAA-1556 |
| — | 16 | 2 | 16 |
| 1 | 16 (2) | 8(2) | 16(2) |
| (9314848) | | | |
| 2 | 16(2) | 8(2) | 16(2) |
| 4 | 16(2) | 8(2) | 64(0.5) |
| 6 | 8(4) | 4(4) | 16(2) |
| 10 | >128(—) | 16(—) | 32(—) |
| 11 | 2(16) | 4(4) | 8(4) |
| 13 | 2(16) | 2(8) | 8(4) |
| 16 | 4(8) | 4(4) | 8(4) |
| 22 | 4(8) | 4(4) | 8(4) |
| 29 | >128(—) | 16(—) | >128(—) |

TABLE 9C
Potentiation of cephalothin by select compounds at 60 mM
against MRSA strains AH-1263, AH-2204, and ATCC BAA-1556.

Cephalothin MIC (μg/mL) (fold reduction)

| | | | ATCC |
|---|---|---|---|
| Compound | AH-1263 | AH-2204 | BAA-1556 |
| — | 16 | 32 | 32 |
| 1 | 1(32) | 1(4) | 4(4) |
| (9314848) | | | |
| 2 | 0.5(64) | 0.5(8) | 1(16) |
| 4 | 2(16) | 0.5(8) | 4(4) |
| 6 | 8(4) | 1(4) | 8(2) |
| 10 | 4(8) | 1(4) | 8(2) |
| 11 | 1(32) | 1(4) | 8(2) |
| 13 | 4(8) | 0.5(8) | 8(2) |
| 16 | 4(8) | 1(4) | 16(—) |
| 22 | 8(4) | 1(4) | 16(1) |
| 29 | 64(0.5) | 16(0.25) | 8(2) |

The majority of compounds were more active as adjuvants when used in combination with a penicillin as compared to a cephalosporin (Tables 4 and 5). It was also noteworthy that compound 11, which was the most potent compound with oxacillin, did not lower the MICs of cefoxitin or amoxicillin by at least four-fold against any MRSA strain. These results highlight the importance of evaluation with a panel of antibiotics to determine both potency and spectrum of activity of these adjuvants.

### Potentiation of Carbapenems by Substituted Phenylureidobenzamide Compounds Against MRSA Strains

Imipenem, a carbapenem to which the MRSA strains used here are susceptible, was also evaluated. Six of ten compounds lowered the MIC by at least four-fold against one or more MRSA strains (Table 10). Compounds 1 (9314848), 2 and 4 were the most potent against strain ATCC BAA-1556, lowering the MIC by 32-fold, 16-fold, and 16-fold, respectively. Compounds 1 (9314848), compounds 2 and 4 were again the most potent against AH-2204, lowering the MIC of imipenem by 8-fold, 16-fold, and 8-fold respectively. No compounds displayed potentiation activity against strain AH-1263. Additionally, compound 29 was inactive against all strains.

TABLE 10
Potentiation of imipenem by select substituted
phenylureidobenzamide compounds at 60 mM against
MRSA strains AH-1263, AH-2204, and ATCC BAA-1556.
Imipenem MIC (μg/mL) (fold reduction)

| | | | ATCC |
|---|---|---|---|
| Compounds | AH-1263 | AH-2204 | BAA-1556 |
| — | 16 | 32 | 32 |
| 1 | 0.062 (2) | 0.031 (8) | 0.015 (32) |
| (9314848) | | | |
| 2 | 4 (0.03) | 0.015 (16) | 0.031 (16) |
| 4 | 2 (0.06) | 0.031 (8) | 0.031 (16) |
| 6 | 0.25 (0.5) | 0.25 (—) | 0.25 (2) |
| 10 | 0.062 (2) | 0.125 (2) | 0.5 (—) |
| 11 | 2 (0.06) | 0.125 (2) | 0.125 (4) |
| 13 | 0.125 (—) | 0.125 (2) | 0.25 (2) |
| 16 | 0.062 (2) | 0.125 (2) | 0.125 (4) |
| 22 | 0.125 (—) | 0.125 (2) | 0.125 (4) |
| 29 | 2 (0.06) | 0.25(—) | >2 (—) |

MRSA Strain Panel for Potentiation Activity with Oxacillin

Compounds 11, 13, and the original lead compound 1 (9314848) were then evaluated against a panel of MRSA strains for their efficacy in potentiating oxacillin (Table 11). Compound 11 was chosen due to its high potency when used with oxacillin, while compound 13 was selected because it was the most potent derivative when used with amoxicillin, cefoxitin and cefotaxime. All compounds lowered the MIC of oxacillin against all strains by at least eight-fold, with the exception of compound 13 against strain BAA-44. Compound 1 (9314848) was the most potent, lowing the MIC by 1024-fold against strain BAA-1770. Against the eleven strains evaluated here, compound 1 (9314848) reduced the MIC by at least 16-fold against nine strains and by at least 64-fold against five strains (Table 11). Compound 11 showed the greatest activity against strain 43300, lowering the MIC of oxacillin by 512-fold. Compound 11 lowered the MIC of oxacillin by at least 32-fold against ten out of eleven MRSA strains and by at least 64-fold against eight strains. Compound 13 was most potent against strain BAA-811, lowering the MIC by 512-fold. Compound 13 lowered the oxacillin MIC by at least 64-fold against nine MRSA strains. Both compounds 11 and 13 were more potent than compound 1 (9314848) against eight of the eleven strains evaluated.

TABLE 11
MRSA strain panel for potentiation activity with oxacillin.
Compounds 1 (9314848), 11, and 13 were tested at 60 mM.

| | Oxacillin MIC (μg/mL) (fold reduction) | | | |
|---|---|---|---|---|
| MRSA | Compounds | | | |
| Strains | — | 1 | 11 | 13 |
| AH-1263 | 16 | 1(16) | 0.125(128) | 0.25(64) |
| AH-2204 | 32 | 1(32) | 0.125 (256) | 0.25 (128) |
| ATCC | 32 | 2(16) | 0.25 (128) | 0.25 (128) |
| BAA-1556 | | | | |
| BAA-811 | 256 | 1 (256) | 2 (128) | 0.5 (512) |
| 700789 | 256 | 4 (64) | 2 (128) | 2 (128) |
| BAA-1770 | 512 | 0.5 (1024) | 4 (128) | 2 (256) |
| 43300 | 512 | 4 (128) | 1 (512) | 2 (256) |
| BAA-1685 | 64 | 8 (8) | 1 (64) | 0.5 (128) |
| 33591 | 128 | 2(64) | 4(32) | 0.5 (256) |
| BAA-44 | 16 | 2 (8) | 2 (8) | 0.5 (256) |
| 700699 | 16 | 1 (16) | 0.5 (32) | 1 (16) |

### Additional Evaluation of Compounds

Dose-response activity of compounds 1 (9314848), 11 and 13 with oxacillin was also determined down to a concentration of 10 μM against ATCC BAA-1556 (Table 12). According to the Clinical and Laboratory Standards Institute (CLSI), the break point of oxacillin is ≤2 μg/mL.

TABLE 12
Dose-responses of compounds 1 (9314848), 11, and 13
with oxacillin against MRSA strain ATCC BAA-1556.
Oxacillin MIC (μg/mL) (fold reduction)

| | Compound concentration (μM) | | |
|---|---|---|---|
| | 1 | 11 | 13 |
| 50 | 1 (32) | 1 (32) | 2 (16) |
| 40 | 32 (—) | 4 (8) | 2 (16) |
| 30 | 32 (—) | 32 (—) | 4 (8) |
| 20 | 32 (—) | 32 (—) | 4 (8) |

Against strain ATCC BAA-1556 the oxacillin MIC was 32 μg/mL. Compounds 1 (9314848) and 11 were inactive below 50 μM, and 13 did not lower the MIC to the break point at concentrations lower than 40 μM. The same panel of MRSA strains described earlier was evaluated at these concentrations, although all compounds were inactive against several strains at the concentration tested (Table 13).

TABLE 13
Potentiation of oxacillin with compounds 1,
11 and 13 at break point concentrations.

| MRSA | Oxacillin MIC (μg/mL) (fold reduction) | | | |
|---|---|---|---|---|
| strain | 1 (50 μM) | 11 (50 μM) | 13 (40 μM) | — |
| AH-1263 | 1 (32) | 1 (32) | 2 (16) | 32 |
| AH-2204 | 4 (8) | 4 (8) | 8 (4) | 32 |
| ATCC | 8 (2) | 32 (—) | 16 (—) | 16 |
| BAA-1556 | | | | |
| BAA-811 | 256 (—) | 128 (2) | 64 (4) | 256 |
| 700789 | 128 (2) | 16 (16) | 128 (2) | 256 |
| BAA-1770 | 128 (4) | 4 (128) | 256 (2) | 512 |
| 4330 | 128 (4) | 8 (64) | 256 (2) | 512 |
| BAA-1685 | 32 (2) | 16 (4) | 32 (2) | 64 |
| 33591 | 128 (—) | 64 (2) | 128 (—) | 128 |
| BAA-44 | 32 (—) | 8 (2) | 8 (2) | 16 |
| 700699 | 16 (—) | 8 (2) | 16 (—) | 16 |

To further quantify activity, time-kill curves were constructed for oxacillin with compounds 11 and 13 as representative lead compounds. Compounds were evaluated against strain ATCC BAA-1556 and both compounds displayed little toxicity toward the bacteria at 60 μM. The combination of either compound with 2 μg/mL oxacillin was determined to be bacteriostatic, as both 11 and 13 showed reduction in the number of viable cells over hours 2-8, followed by a gradual rise in viable cells to hour 24. Compounds were also evaluated with oxacillin at MIC values (0.25 μg/mL). Compound 11 showed a decrease in viable cells after 2 hours and an increase from hours 4-24 while 13 showed a decrease through 8 hours.

### Example 3: Benzimidazole-Based Small Molecules Targeting PBP4

### Abbreviations

DCM for dichloromethane;
MeOH is methanol;
Pd/C is palladium on carbon;
TEA is triethylamine;
NaHCO₃ is sodium bicarbonate;
TLC is thin-layer chromatography;
EtOAc is ethyl acetate;
Hex is hexanes;
HPLC is high-performance liquid chromatography;
TFA is trifluoracetic acid;
ACN is acetonitrile;
min or min. is minute(s);
h or hr. is hour(s);
rt, RT, or r.t. is room temperature;
and sat. is saturated;

### Synthesis of Compounds

### Synthesis of Example Intermediates

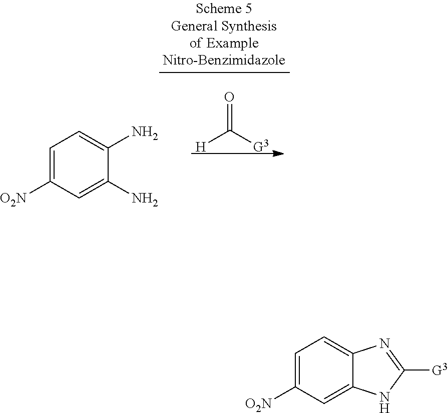

A 250-mL round-bottomed flask equipped with a magnetic stirring bar, and an N₂ inlet was charged with diamine (1.5 g, 9.8 mmol) in 70 mL water. To the mixture, carbaldehyde (1.5 g, 4.9 mmol) was added and the stirring reaction was heated to 50° C. The respective aldehyde (1.1 g, 9.8 mmol) was then added slowly with stirring and the mixture stirred for 12 h at 50° C. The resulting mixture was the cooled to room temperature and extracted several times with ethyl acetate. The organic layers were combined and washed with saturated NaHCO₃ (3×20 mL), with water (3×20 mL) and with brine (3×20 mL). The organic layer was dried with MgSO₄ and concentrated in vacuo. Purification of the residue by chromatography on silica gel (EtOAc/Hex for elution) provided the title compound.

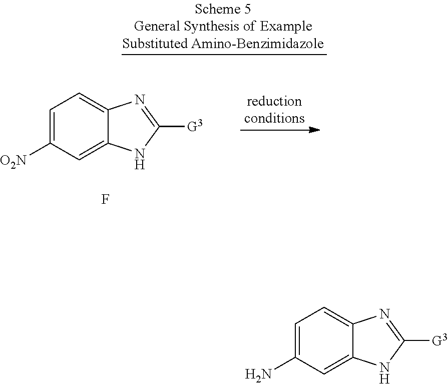

A 100-mL round-bottomed flask was equipped with a magnetic stirring bar and charged with Pd/C 50% wet (43 mg, 0.4 mmol). The flask was then purged with H₂ and nitro-benzimidazole (500 mg, 2.0 mmol) was dissolved and added to the reaction mixture. The reaction mixture was stirred at room temperature until completion. The mixture was filtered and concentrated to afford the compound.

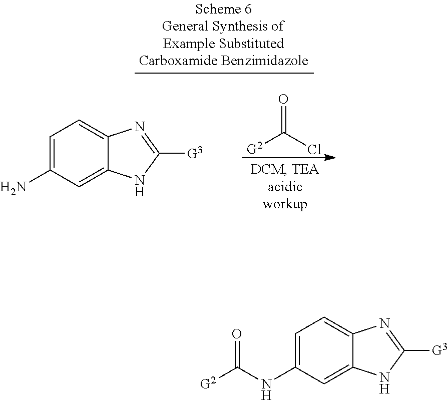

To aflame dried 100-mL round-bottomed flask, the resulting amino-benzimidazole (120 mg, 0.557 mmol) from the above procedure was added dissolved in DCM (8 mL). To the mixture, TEA (124 mg, 1.23 mmol) was added with stirring. The reaction mixture stirred for 5 minutes before the corresponding acid chloride (97.6 mg, 0.557 mmol) was added slowly. The reaction was stirred at room temperature until completion. The reaction mixture was quenched with 2M HCl, diluted with water and extracted with DCM (3×10 mL). The organic layers were combined and dried with MgSO₄ and concentrated in vacuo. Purification was accomplished by chromatography on silica gel (EtOAc/Hex for elution) or via recrystallization in DCM. If necessary, purification by HPLC was also employed after initial purification, using the gradient 15-80% ACN and water with 1% TFA to provide the title compound.

### Example Compounds

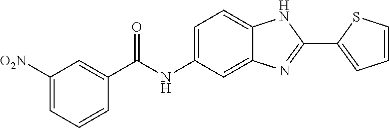 [SMILES: O=C(Nc1ccc2[nH]c(-c3cccs3)nc2c1)c1cccc([N+](=O)[O-])c1]

3-Nitro-N-(2-(thiophen-2-yl)-1H-benzo[d]imidazol-5-yl)benzamide (6). Using the general procedures outlined above, 6 was synthesized as a yellow solid in 74% yield (208 mg, 571 mmol). ¹H NMR (400 MHz, Methanol-d₄) δ=8.84 (d, J=4.25 Hz, 1H), 7.34 (dd, J=2.35, 7.65 Hz, 2H), 8.37 (s, 1H), 8.16 (s, 1H), 8.05 (d, J=5.70 Hz, 1H), 7.88 (t, J=10.00 Hz, 1H), 7.75-7.74 (m, 2H), 7.39 (t, J=5.35 Hz, 1H). LRMS: (ESI) calculated for C₁₈H₁₂N₄O₃S⁺ ([M+H]⁺) 365.0703, found 365.0696 M/Z. HPLC trace: 95.6%. Commercially available through AKos Consulting & Solutions GmbH (ID: 007301286).

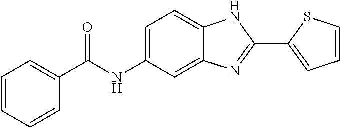 [SMILES: O=C(Nc1ccc2[nH]c(-c3cccs3)nc2c1)c1ccccc1]

N-(2-(Thiophen-2-yl)-1H-benzo[d]imidazol-5-yl)benzamide (19). Using the general procedures outlined above, 19 was synthesized as a white solid in 64% yield (205 mg, 0.54 mmol). ¹H NMR (400 MHz, DMSO-d₆) δ=8.07 (d, J=9.05 Hz, 2H), 7.92 (d, J=8.95 Hz, 2H), 7.65 (t, J=9.40 Hz, 1H), 7.52-7.48 (m, 4H), 7.38 (t, J=10.00 Hz, 2H). LRMS: (ESI) for C₁₈H₁₃N₃OS⁺ ([M+H]⁺), 320.3119 M/Z. HPLC trace: 95.3%. Commercially available through Aurora Fine Chemicals LLC (ID: K02.873.478).

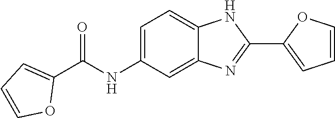 [SMILES: O=C(Nc1ccc2[nH]c(-c3ccco3)nc2c1)c1ccco1]

N-(2-(Furan-2-yl)-1H-benzo[d]imidazol-5-yl)furan-2-carboxamide (24). Using the general procedures outlined above, 24 was synthesized as a yellow solid in 5% yield (8 mg, 0.03 mmol). ¹H NMR (400 MHz, Methanol-d₄) δ=8.22-8.16 (m, 1H), 8.14 (d, J=1.35 Hz, 1H), 7.77 (s, 1H), 7.67 (t, J=4.50 Hz, 1H), 7.35-7.32 (m, 1H), 7.23 (dd, J=2.35 Hz, 1H), 7.08-7.06 (m, 1H), 6.61-6.60 (m, 1H), 6.54-6.45 (m, 1H). LRMS: (ESI) calculated for C₁₆H₁₁N₃O₃⁺ ([M+H]⁺) 294.0873, found 294.0879 M/Z. HPLC trace: 96.9%. Previously published in Chem. Abstr., 1970, vol. 72, #90461q.

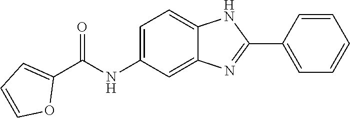 [SMILES: O=C(Nc1ccc2[nH]c(-c3ccccc3)nc2c1)c1ccco1]

N-(2-Phenyl-1H-benzo[d]imidazol-5-yl)furan-2-carboxamide (28). Using the general procedures outlined above, 28 was synthesized as a yellow solid in 9% yield (13 mg, 0.043 mmol). ¹H NMR (400 MHz, CDCl₃) δ=8.33 (d, J=8.80 Hz, 1H), 8.18 (s, 1H), 8.05 (d, J=10.80 Hz, 1H), 7.83 (dd, J=11.20, 11.15 Hz, 1H), 7.67 (t, J=9.55 Hz, 1H), 7.48-7.45 (m, 2H), 7.31-7.29 (m, 2H), 7.05 (d, J=8.5 Hz, 2H). LRMS: (ESI) calculated for C₁₈H₁₃N₃O₂⁺ ([M+H]⁺) 304.1081, found 304.1052 M/Z. HPLC trace: 97.2%. Commercially available through Aurora Fine Chemicals (ID: K00.343.346).

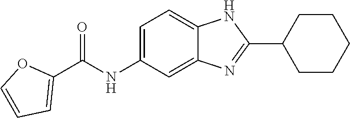 [SMILES: O=C(Nc1ccc2[nH]c(C3CCCCC3)nc2c1)c1ccco1]

N-(2-Cyclohexyl-1H-benzo[d]imidazol-5-yl)furan-2-carboxamide (34). Using the general procedures outlined above, 34 was synthesized as a white solid in 83% yield (109 mg, 352 mmol). ¹H NMR (400 MHz, CDCl₃) δ=8.47 (s, 1H), 7.78-7.74 (m, 3H), 7.34 (d, J=4.35 Hz, 1H), 6.70 (d, J=4.3 Hz, 1H), 3.18-3.14 (m, 1H), 2.25 (d, J=13.20 Hz, 2H), 2.00 (d, J=17.35 Hz, 2H), 1.87 (d, J=16.25 Hz, 1H), 1.74 (t, J=4.25 Hz, 2H), 1.57 (t, J=15.95 Hz, 2H), 7.46-7.44 (m, 1H). LRMS: (ESI) for C₁₈H₁₉N₃O₂⁺ ([M+H]⁺) 310.1550 M/Z. HPLC trace: 95.4%. Commercially available through Aurora Fine Chemicals (ID:191.922.007).

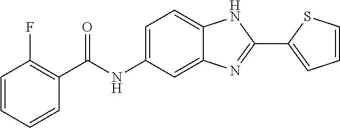 [SMILES: O=C(Nc1ccc2[nH]c(-c3cccs3)nc2c1)c1ccccc1F]

2-Fluoro-N-(2-(thiophen-2-yl)-1H-benzo[d]imidazol-5-yl)benzamide (2). Using the general procedures outlined above, 2 was synthesized as a dark yellow solid in 66% yield (134 mg, 0.397 mmol). ¹H NMR (400 MHz, Methanol-d₄) δ=8.58 (s, 1H), 8.14 (dd, J=4.80, 4.55 Hz, 2H), 7.80-7.76 (m, 2H), 7.71 (d, J=8.85 Hz, 1H), 7.64-7.62 (m, 1H), 7.46 (t, J=4.90 Hz, 1H), 7.39-7.31 (m, 2H). ¹³C NMR (500 MHz, Methanol-d₄) δ=164.40 (C), 137.47 (C), 133.85 (C), 133.07 (C), 132.99 (C), 132.62 (C), 132.01 (C), 129.89 (CH), 129.20 (CH), 128.14 (CH), 124.41 (CH), 123.86 (CH), 119.59 (CH), 116.08 (C), 115.86 (CH), 113.56 (CH), 104.20 (CH). HRMS (ESI) calculated for C₁₈H₁₃FN₃OS⁺ ([M+H]⁺) 338.0758, found 338.0753 M/Z. IRᵥₘₐₓ (cm⁻¹): 2738.25, 1638.10, 1565.38, 716.11. UV (λₘₐₓ nm)=330. HPLC trace: 95.2%.

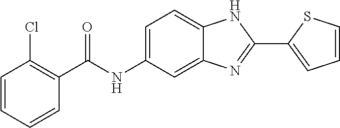 [SMILES: O=C(Nc1ccc2[nH]c(-c3cccs3)nc2c1)c1ccccc1Cl]

2-Chloro-N-(2-(thiophen-2-yl)-1H-benzo[d]imidazol-5-yl)benzamide (3). Using the general procedures outlined above, 3 was synthesized as a white solid in 18% yield (36 mg, 0.100 mmol). ¹H NMR (400 MHz, Methanol-d₄) δ=8.28-8.27 (m, 2H), 7.94 (d, J=4.75 Hz, 1H), 7.59 (d, J=8.80 Hz, 1H), 7.31-7.29 (m, 4H), 7.28-7.20 (m, 2H). ¹³C NMR (500 MHz, DMSO-d₆): δ=168.41 (C), 158.75 (C), 147.24 (C), 146.25 (C), 136.18 (C), 134.42 (C), 134.18 (C), 132.75 (CH), 129.51 (CH), 129.15 (C), 129.11 (CH), 127.57 (CH), 126.24 (CH), 122.74 (CH), 121.30 (CH), 119.03 (CH), 116.68 (CH). HRMS (ESI) calculated for C₁₈H₁₃ClN₃OS⁺ ([M+H]⁺), 354.8240, found, 354.0462 M/Z. IRᵥₘₐₓ (cm⁻¹): 3245, 1659, 1178, 743. UV (λₘₐₓ nm)=336. HPLC trace: 97.7%.

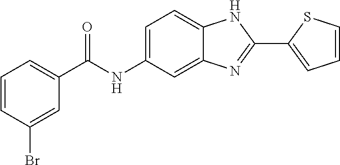 [SMILES: O=C(Nc1ccc2[nH]c(-c3cccs3)nc2c1)c1cccc(Br)c1]

3-Bromo-N-(2-(thiophen-2-yl)-1H-benzo[d]imidazol-5-yl)benzamide (4). Using the general procedures outlined above, 4 was synthesized as a yellow solid in 23% yield (46 mg, 0.12 mmol). ¹H NMR (400 MHz, Methanol-d₄) δ=8.33 (s, 1H), 7.93 (d, J=3.50 Hz, 1H), 7.88-7.85 (m, 3H), 7.69 (d, J=9.95 Hz, 1H), 7.59 (s, 2H), 7.38 (t, J=7.88 Hz, 1H), 7.28 (t, J=3.92 Hz, 1H). ¹³C NMR (500 MHz, DMSO-d₆): δ=165.83 (C), 145.41 (C), 136.82 (C), 136.74 (C), 134.59 (C), 133.23 (C), 132.66 (C), 131.40 (CH), 130.34 (C), 130.17 (C), 129.95 (C), 128.90 (CH), 126.12 (CH), 125.63 (CH), 122.21 (CH), 119.41 (CH), 113.71 (CH), 105.06 (CH). HRMS (ESI) calculated for C₁₈H₁₃BrN₃OS⁺ ([M+H]⁺) 399.9957, 397.9945 M/Z. IRᵥₘₐₓ (cm-1): 3068, 1669, 1010, 717. UV (λₘₐₓ nm)=336. HPLC trace: 95.7%.

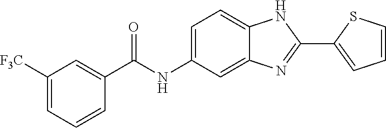 [SMILES: O=C(Nc1ccc2[nH]c(-c3cccs3)nc2c1)c1cccc(C(F)(F)F)c1]

N-(2-(Thiophen-2-yl)-1H-benzo[d]imidazol-5-yl)-3-(trifluoromethyl)benzamide (5). Using the general procedures outlined above, 5 was synthesized as a beige solid in 47% yield (89 mg, 0.23 mmol). ¹H NMR (400 MHz, CDCl3-d₄) δ=8.33 (d, J=10.55 Hz, 1H), 8.24 (d, J=9.80 Hz, 1H), 8.18 (s, 1H), 8.03-8.01 (m, 3H), 7.99-7.84 (m, 2H), 7.70 (s, 1H), 7.36 (t, J=5.75 Hz, 1H). ¹³C NMR (500 MHz, DMSO-d₆) δ=164.40 (C), 145.33 (C), 136.70 (C), 136.09 (C), 133.71 (C), 132.35 (C), 130.60 (C), 130.26 (CH), 129.92 (C), 129.88 (CH), 129.50 (CH), 125.98 (CH), 125.95 (CH), 124.84 (CH), 122.91 (CH), 119.15 (C), 114.78 (CH), 105.23 (CH). HRMS (ESI) calculated for C₁₉H₁₃F₃N₃OS⁺ ([M+H]⁺), 388.0653, found, 388.0876 M/Z. IRᵥₘₐₓ (cm⁻¹): 2546.04, 1637.51, 1483.28, 856.72. UV (λₘₐₓ nm)=328. HPLC trace: 96.5%.

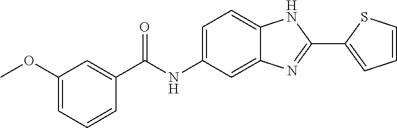 [SMILES: COc1cccc(C(=O)Nc2ccc3[nH]c(-c4cccs4)nc3c2)c1]

3-Methoxy-N-(2-(thiophen-2-yl)-1H-benzo[d]imidazol-5-yl)benzamide (7). Using the general procedures outlined above, 7 was synthesized as a dark yellow solid in 73% yield (51 mg, 0.15 mmol). ¹H NMR (400 MHz, CDCl₃) δ=8.30 (s, 1H), 8.02-7.95 (m, 2H), 7.67-7.59 (m, 2H), 7.58 (d, J=9.50 Hz, 1H), 7.52 (s, 1H), 7.47 (t, J=9.75 Hz, 2H), 7.35-7.34 (m, 1H), 7.19 (dd, J=2.55, 3.00 Hz, 1H), 3.86 (s, 3H). ¹³C NMR (500 MHz, DMSO-d₆) δ=165.91 (C), 159.68 (C), 136.74 (C), 134.73 (C), 133.42 (C), 133.08 (C), 130.93 (C), 130.09 (C), 129.76 (CH), 129.38 (C), 128.66 (CH), 128.23 (CH), 120.38 (CH), 118.66 (CH), 117.87 (CH), 113.76 (CH), 113.43 (CH), 55.85 (CH₃). HRMS (ESI) calculated for C₁₉H₁₆N₃O₂S⁺ ([M+H]⁺), 350.0885, found, 350.0958 M/Z. IRᵥₘₐₓ (cm⁻¹): 2636.12, 2543.16, 1566.28, 807.78. UV (λₘₐₓ nm)=322. HPLC trace: 97.7%.

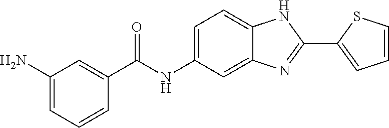 [SMILES: Nc1cccc(C(=O)Nc2ccc3[nH]c(-c4cccs4)nc3c2)c1]

3-Amino-N-(2-(thiophen-2-yl)-1H-benzo[d]imidazol-5-yl)benzamide (8). Using the general procedures outlined above, 8 was synthesized as a beige solid in 96% yield (75 mg, 0.22 mmol). ¹H NMR (400 MHz, DMSO-d₆) δ=8.90 (s, 1H), 8.59 (d, J=2.35 Hz, 2H), 8.46 (s, 1H), 8.26-8.17 (m, 1H), 8.04-8.03 (m, 1H), 7.89 (t, J=5.20 Hz, 1H), 7.77-7.74 (m, 2H), 7.39 (t, J=9.00 Hz, 1H). ¹³C NMR (500 MHz, Methanol-d₄) δ=159.98 (C), 155.69 (C), 149.22 (C), 146.73 (C), 144.44 (C), 137.00 (C), 134.52 (C), 133.70 (CH), 132.02 (CH), 129.26 (C), 128.11 (CH), 127.19 (CH), 126.85 (CH), 124.80 (CH), 123.97 (CH), 119.15 (CH), 114.23 (CH), 111.11 (CH). HRMS (ESI) calculated for C₁₈H₁₅N₄OS⁺ ([M+H]⁺) 335.0961, 335.0951 M/Z. IRᵥₘₐₓ (cm⁻¹): 2583.58, 1613.74, 1232.80, 809.50. UV (λₘₐₓ nm)=342. HPLC trace: 99.2%.

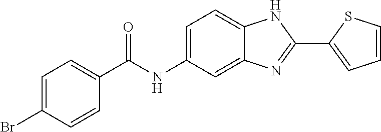 [SMILES: O=C(Nc1ccc2[nH]c(-c3cccs3)nc2c1)c1ccc(Br)cc1]

4-Bromo-N-(2-(thiophen-2-yl)-1H-benzo[d]imidazol-5-yl)benzamide (9). Using the general procedures outlined above, 9 was synthesized as a beige solid in 82% yield (88 mg, 0.22 mmol). ¹H NMR (400 MHz, DMSO-d₆) δ=8.43 (s, 1H), 8.30 (s, 1H), 8.13-8.10 (m, 3H), 8.02 (s, 1H), 7.73 (d, J=8.88 Hz, 2H), 7.36 (s, 1H), 6.78-6.77 (m, 1H). ¹³C NMR (500 MHz, DMSO-d₆) δ=165.29 (C), 148.24 (C), 146.39 (C), 144.88 (C), 143.22 (C), 137.13 (C), 131.44 (CH), 130.46 (CH), 128.48 (CH), 126.50 (CH), 123.78 (CH), 122.80 (C), 118.63 (CH), 113.22 (CH), 111.92 (CH). HRMS (ESI) calculated for C₂₀H₁₈N₃OS⁺ ([M+H]⁺), 348.1092, found, 348.9986 M/Z. IRᵥₘₐₓ (cm⁻¹): 3129.52, 2659.23, 1654.27, 1354.33. UV (λₘₐₓ nm)=348. HPLC trace: 98.7%.

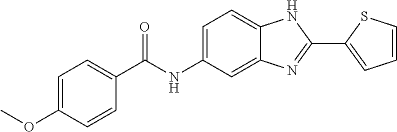 [SMILES: COc1ccc(C(=O)Nc2ccc3[nH]c(-c4cccs4)nc3c2)cc1]

4-Methoxy-N-(2-(thiophen-2-yl)-1H-benzo[d]imidazol-5-yl)benzamide (10). Using the general procedures outlined above, 10 was synthesized as a yellow solid in 12% yield (21 mg, 0.060 mmol). H NMR (400 MHz, Methanol-d₄) δ=8.21 (s, 1H), 7.89 (d, J=3.70 Hz, 2H), 7.85 (d, J=5.80 Hz, 1H), 7.56 (d, J=8.84 Hz, 1H), 7.39-7.32 (m, 4H), 7.26-7.20 (m, 2H), 3.84 (s, 3H). ¹³C NMR (500 MHz, CDCl₃): δ=166.77 (C), 160.84 (C), 154.67 (C), 149.23 (C), 138.36 (C), 136.38 (C), 133.67 (C), 131.19 (CH), 128.97 (CH), 126.25 (CH), 123.28 (CH), 119.09 (CH), 116.12 (CH), 114.89 (CH), 112.91 (CH), 109.94 (CH), 57.57 (CH₃). HRMS: (ESI) calculated for C₁₉H₁₆N₃O₂S⁺ ([M+H]⁺) 350.0958, found 350.0933 M/Z. IRᵥₘₐₓ (cm⁻¹): 2826.74, 2552.01, 1615.09, 778.52. UV (λₘₐₓ nm)=326. HPLC trace: 96.3%.

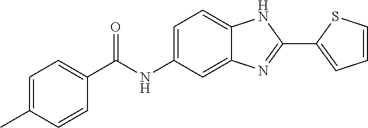 [SMILES: Cc1ccc(C(=O)Nc2ccc3[nH]c(-c4cccs4)nc3c2)cc1]

4-Methyl-N-(2-(thiophen-2-yl)-1H-benzo[d]imidazol-5-yl)benzamide (11). Using the general procedures outlined above, 11 was synthesized as a white solid in 56% yield (431 mg, 1.29 mmol). ¹H NMR (400 MHz, Methanol-d₄) δ=8.22 (s, 1H), 7.88 (dd, J=3.70, 6.15 Hz, 1H), 7.54 (d, J=11.00 Hz, 1H), 7.37 (dd, J=2.25, 2.30 Hz, 1H), 7.26 (t, J=4.95 Hz, 1H), 7.20 (d, J=10.75 Hz, 1H), 7.08 (d, J=10.75 Hz, 2H), 6.81-6.75 (m, 2H), 3.68 (d, J=5.00 Hz, 3H). ¹³C NMR (500 MHz, Methanol-d₄): δ=165.91 (C), 140.79 (C), 139.57 (C), 136.99 (C), 136.10 (C), 132.31 (C), 130.86 (C), 129.12 (CH), 128.87 (C), 127.53 (CH), 127.33 (C), 126.56 (CH), 126.30 (CH), 113.89 (CH), 108.52 (CH), 31.86 (CH₃). HRMS (ESI) calculated for C₁₉H₁₆N₃OS⁺ ([M+H]⁺) 334.1009, 334.1002 M/Z. IRᵥₘₐₓ (cm⁻¹): 3075, 1664, 1184, 718. UV (λₘₐₓ nm)=334. HPLC trace: 98.1%.

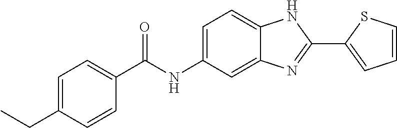 [SMILES: CCc1ccc(C(=O)Nc2ccc3[nH]c(-c4cccs4)nc3c2)cc1]

4-Ethyl-N-(2-(thiophen-2-yl)-1H-benzo[d]imidazol-5-yl)benzamide (12). Using the general procedures outlined above, 12 was synthesized as a white solid in 20% yield (26 mg, 0.075 mmol). ¹H NMR (400 MHz, Methanol-d₄) δ=8.45 (s, 1H), 8.05 (d, J=4.05 Hz, 2H), 8.00 (d, J=6.05 Hz, 2H), 7.92 (d, J=10.30 Hz, 2H), 7.71 (s, 1H), 7.42-7.39 (d, J=4.75 Hz, 2H), 2.78 (q, J=9.75, 9.40 Hz, 2H), 1.33 (t, J=9.50 Hz, 3H). ¹³C NMR (500 MHz, Methanol-d₄): δ=165.75 (C), 149.98 (C), 148.65 (C), 146.35 (C), 138.33 (C), 136.74 (C), 133.58 (CH), 132.08 (CH), 129.51 (CH), 127.62 (CH), 127.05 (CH), 118.74 (CH), 116.24 (CH), 114.02 (CH), 28.15 (CH₂), 15.82 (CH₃). HRMS: (ESI) calculated for C₂₀H₁₈N₃OS⁺ ([M+H]⁺) 348.1165, found 348.1168 M/Z. IRᵥₘₐₓ (cm⁻¹): 2869, 1725, 1109, 718. UV (λₘₐₓ nm)=338. HPLC trace: 97.8%.

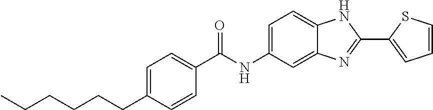 [SMILES: CCCCCCc1ccc(C(=O)Nc2ccc3[nH]c(-c4cccs4)nc3c2)cc1]

4-Hexyl-N-(2-(thiophen-2-yl)-1H-benzo[d]imidazol-5-yl)benzamide (13). Using the general procedures outlined above, 13 was synthesized as a white solid in 43% yield (21 mg, 0.052 mmol). ¹H NMR (400 MHz, CDCl₃) δ=8.65 (s, 1H), 8.16-8.13 (m, 1H), 7.92-7.90 (m, 1H), 7.84 (d, J=11.05 Hz, 1H), 7.63 (dd, J=5.20, 2.00 Hz, 2H), 7.45-7.30 (m, 1H), 7.11 (dd, J=4.40, 4.40 Hz, 2H), 6.93-6.39 (m, 1H), 2.61-2.59 (m, 2H), 1.51-1.46 (m, 6H), 1.25-1.21 (m, 3H), 0.83-0.81 (m, 2H). ¹³C NMR (500 MHz, Methanol-d₄): δ=150.19 (C), 146.41 (C), 137.14 (C), 135.83 (C), 133.15 (C), 132.42 (C), 130.20 (C), 128.27 (CH), 127.23 (CH), 126.51 (CH), 125.66 (CH), 125.60 (CH), 124.64 (CH), 118.54 (CH), 113.44 (CH), 113.19 (CH), 106.28 (CH), 42.11 (CH₂), 39.68 (CH₂), 29.38 (CH₂), 25.38 (CH₂), 21.69 (CH), 13.03 (CH₃). HRMS (ESI) calculated for C₂₄H₂₅N₃OS⁺ ([M+H]˜) 404.1791, found 404.1789 M/Z. IRᵥₘₐₓ (cm⁻¹): 3026, 1710, 1220, 712. UV (λₘₐₓ nm)=346. HPLC trace: 97.5%.

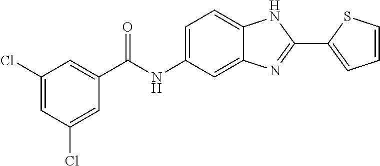 [SMILES: O=C(Nc1ccc2[nH]c(-c3cccs3)nc2c1)c1cc(Cl)cc(Cl)c1]

3,5-Dichloro-N-(2-(thiophen-2-yl)-1H-benzo[d]imidazol-5-yl)benzamide (14). Using the general procedures outlined above, 14 was synthesized as a white solid in 31% yield (96 mg, 0.250 mmol). ¹H NMR (400 MHz, CDCl₃) δ=8.23 (s, 1H), 8.10 (d, J=7.8 Hz, 1H), 7.89 (dd, J=4.52 Hz, 2H), 7.84 (d, J=8.08 Hz, 1H), 7.79-7.74 (m, 2H), 7.70 (d, J=4.12 Hz, 1H), 7.21 (t, J=4.36 Hz, 1H), 7.03 (t, J=4.88 Hz, 1H). ¹³C NMR (500 MHz, DMSO-d₆) δ=163.08 (C), 154.60 (C), 147.77 (C), 140.07 (C), 139.60 (C), 136.88 (C), 136.10 (C), 132.28 (C), 131.58 (CH), 130.80 (CH), 129.94 (CH), 129.55 (CH), 127.21 (CH), 126.41 (CH), 119.15 (CH), 117.48 (CH). HRMS (ESI) calculated for C₁₈H₁₂Cl₂N₃OS⁺ ([M+H]⁺) 388.0073, found 388.0059 M/Z. IRᵥₘₐₓ (cm⁻¹): 2922, 1667, 1442, 857. UV (λₘₐₓ nm)=282. HPLC trace: 96.8%.

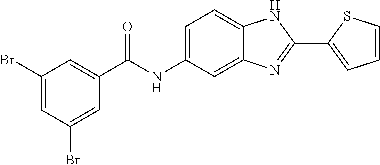 [SMILES: O=C(Nc1ccc2[nH]c(-c3cccs3)nc2c1)c1cc(Br)cc(Br)c1]

3,5-Dibromo-N-(2-(thiophen-2-yl)-1H-benzo[d]imidazol-5-yl)benzamide (15). Using the general procedures outlined above, 15 was synthesized as a white solid in 56% yield (431 mg, 1.29 mmol). ¹H NMR (400 MHz, Methanol-d₄) δ=8.40 (s, 1H), 8.08-7.86 (m, 4H), 7.68 (d, J=10.75 Hz, 1H), 7.49 (s, 1H), 7.41 (t, J=6.16 Hz, 1H), 7.28 (d, J=3.85 Hz, 1H). ¹³C NMR (500 MHz, DMSO-d₆): δ=166.40 (C), 145.02 (C), 139.28 (C), 137.84 (C), 136.80 (C), 132.02 (C), 129.26 (C), 127.19 (CH), 126.85 (C), 125.62 (C), 124.59 (C), 122.93 (CH), 121.90 (C), 119.15 (CH), 114.23 (CH), 111.11 (CH). HRMS (ESI) calculated for C₁₈H₁₂Br₂N₃OS⁺ ([M+H]⁺), 475.8990, found, 475.9957 M/Z. IRᵥₘₐₓ (cm⁻1): 2998, 1696, 1210, 783. UV (λₘₐₓ nm)=324. HPLC trace: 96.8%.

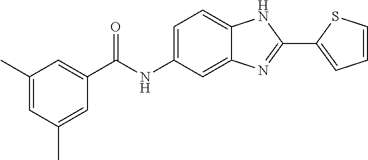 [SMILES: Cc1cc(C)cc(C(=O)Nc2ccc3[nH]c(-c4cccs4)nc3c2)c1]

3,5-Dimethyl-N-(2-(thiophen-2-yl)-1H-benzo[d]imidazol-5-yl)benzamide (16). Using the general procedures outlined above, 16 was synthesized as a white solid in 68% yield (68 mg, 0.20 mmol). ¹H NMR (400 MHz, DMSO-d₆) δ=8.33 (s, 1H), 8.07-7.99 (m, 1H), 7.70-7.66 (m, 1H), 7.60 (s, 1H), 7.56 (s, 1H), 7.36 (d, J=5.10 Hz, 1H), 7.25 (s, 1H), 2.38 (s, 1H), 2.32 (s, 1H). ¹³C NMR (500 MHz, DMSO-d₆): δ=164.78 (C), 152.82 (C), 143.26 (C), 138.65 (C), 137.25 (C), 136.95 (C), 134.26 (C), 133.66 (C), 132.92 (CH), 128.81 (C), 127.85 (CH), 127.56 (CH), 127.09 (CH), 119.52 (CH), 116.32 (CH), 115.64 (CH), 22.89 (CH6). HRMS: (ESI) calculated for C₂₀H₁₈N₃OS⁺ ([M+H]⁺), 348.4360, found, 348.0239 M/Z. IRᵥₘₐₓ (cm⁻¹): 2988, 1665, 1112, 756. UV (λₘₐₓ nm)=318. HPLC trace: 95.8%.

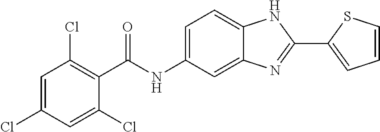 [SMILES: O=C(Nc1ccc2[nH]c(-c3cccs3)nc2c1)c1c(Cl)cc(Cl)cc1Cl]

2,4,6-Trichloro-N-(2-(thiophen-2-yl)-1H-benzo[d]imidazol-5-yl)benzamide (17). Using the general procedures outlined above, 17 was synthesized as a white solid in 39% yield (208 mg, 0.492 mmol). ¹H NMR (400 MHz, Methanol-d₄) δ=8.01 (s, 1H), 7.75 (d, J=3.52 Hz, 1H), 7.72 (d, J=4.96 Hz, 1H), 7.61 (dd, J=8.84 Hz, 1H), 7.56 (s, 2H), 7.54 (dd, J=3.08 Hz, 1H), 7.42 (t, J=4.96 Hz, 1H). ¹³C NMR (500 MHz, DMSO-d₆): δ=161.73 (C), 158.82 (C), 155.17 (C), 146.83 (C), 135.84 (C), 135.57 (C), 135.29 (C), 135.06 (C), 132.66 (CH), 131.18 (C), 129.15 (CH), 128.87 (CH), 128.59 (CH), 118.45 (CH), 117.68 (CH), 116.70 (CH), 115.97 (CH). HRMS (ESI) calculated for C₁₈H₁₁Cl₃N₃OS⁺ ([M+H]⁺) 421.9683, 421.9674 M/Z. IRᵥₘₐₓ (cm¹): 3077, 1659, 1183, 717. UV (λₘₐₓ nm)=332. HPLC trace: 95.0%.

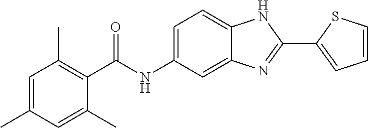 [SMILES: Cc1cc(C)c(C(=O)Nc2ccc3[nH]c(-c4cccs4)nc3c2)c(C)c1]

2,4,6-Trimethyl-N-(2-(thiophen-2-yl)-1H-benzo[d]imidazol-5-yl)benzamide (18). Using the general procedures outlined above, 18 was synthesized as a white solid in 56% yield (431 mg, 1.29 mmol). ¹H NMR (400 MHz, Methanol-d₄) δ=8.45 (s, 1H), 7.97 (dd, J=3.5, 6.2 Hz, 2H), 7.61 (d, J=11.00 Hz, 1H), 7.46 (d, J=2.35 Hz, 1H), 7.30 (t, J=6.10 Hz, 1H), 6.86 (s, 1H), 2.24 (s, 6H), 2.21 (s, 3H). ¹³C NMR (500 MHz, DMSO-d₆): δ=163.10 (C), 155.87 (C), 138.85 (C), 137.05 (C), 134.85 (C), 133.90 (C), 132.97 (C), 131.70 (C), 128.97 (CH), 128.50 (CH), 127.88 (C), 125.23 (C), 118.72 (CH), 114.93 (C), 113.81 (CH), 105.38 (CH), 104.08 (CH), 19.80 (CH₆), 17.78 (CH₃). HRMS (ESI) calculated for C₂₁H₂₀N₃OS⁺ ([M+H]⁺) 362.1391, found 362.1326 M/Z. IRᵥₘₐₓ (cm⁻¹): 3071, 1651, 1075, 710. UV (λₘₐₓ nm)=348. HPLC trace: 98.6%.

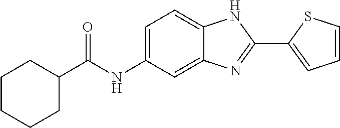 [SMILES: O=C(Nc1ccc2[nH]c(-c3cccs3)nc2c1)C1CCCCC1]

N-(2-(Thiophen-2-yl)-1H-benzo[d]imidazol-5-yl)cyclohexanecarboxamide (20). Using the general procedures outlined above, 20 was synthesized as a white solid in 18% yield (21 mg, 0.065 mmol). ¹H NMR (400 MHz, Methanol-d₄) δ=8.24 (dd, J=3.6, 6.2 Hz, 2H), 7.90 (d, J=11.00 Hz, 2H), 7.53 (d, J=11.15 Hz, 1H), 7.35 (d, J=6.92 Hz, 1H), 7.27 (t, J=5.00 Hz, 1H), 2.31-2.28 (m, 2H), 1.81-1.77 (m, 2H), 1.72-1.65 (m, 2H), 1.46-1.42 (m, 2H), 1.29-1.20 (m, 2H), 0.81-0.77 (m, 1H). ¹³C NMR (500 MHz, Methanol-d₄): δ=176.57 (C), 145.15 (C), 137.21 (C), 133.23 (C), 132.49 (C), 131.22 (C), 128.86 (C), 118.49 (CH), 113.72 (CH), 103.92 (CH), 45.84 (CH), 29.30 (CH₂), 25.47 (CH₂), 25.37 (CH₂). HRMS (ESI) calculated for C₁₈H₂₀N₃OS⁺ ([M+H]⁺) 326.1322, found 326.1315 M/Z. IRᵥₘₐₓ (cm⁻¹): 3083.38, 1660.62, 1181.31, 718.09. UV (λₘₐₓ nm)=312. HPLC trace: 98.0%.

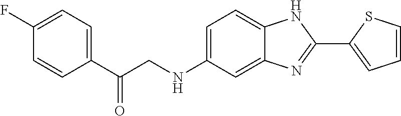 [SMILES: O=C(CNc1ccc2[nH]c(-c3cccs3)nc2c1)c1ccc(F)cc1]

1-(4-Fluorophenyl)-2-((2-(thiophen-2-yl)-1H-benzo[d]imidazol-5-yl)amino)ethan-1-one (21). Using the general procedures outlined above, 21 was synthesized as a yellow solid in 55% yield (88 mg, 0.250 mmol). ¹H NMR (400 MHz, CDCl₃) δ=8.13 (s, 1H), 7.90-7.86 (m, 2H), 7.57-7.56 (m, 1H), 7.39-7.28 (m, 4H), 7.17 (t, J=11.15 Hz, 2H), 3.68 (s, 2H). ¹³C NMR (500 MHz, CDCl₃) δ=183.58 (C), 167.02 (C), 149.73 (C), 148.61 (C), 147.42 (C), 146.08 (C), 145.03 (CH), 141.32 (C), 140.67 (C), 137.40 (CH), 136.97 (CH), 136.27 (CH), 136.04 (CH), 135.20 (CH), 125.06 (CH), 120.34 (CH), 54.11 (CH₂). HRMS (ESI) calculated for C₁₉H₁₅FN₃OS⁺ ([M+H]⁺) 352.0914, 352.0912 M/Z. IRᵥₘₐₓ (cm⁻¹): 2638.85, 1631.86, 1507.98, 1223.08. UV (λₘₐₓ nm)=328. HPLC trace: 99.4%.

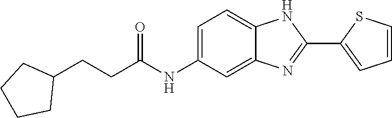 [SMILES: O=C(CCC1CCCC1)Nc1ccc2[nH]c(-c3cccs3)nc2c1]

3-Cyclopentyl-N-(2-(thiophen-2-yl)-1H-benzo[d]imidazol-5-yl)propenamide (22). Using the general procedures outlined above, 22 was synthesized as a yellow solid in 74% yield (208 mg, 571 mmol). ¹H NMR (400 MHz, CDCl₃) δ=7.92 (d, J=4.20 Hz, 1H), 7.79 (d, J=11.00 Hz, 1H), 7.70 (d, J=6.20 Hz, 1H), 7.25 (t, J=4.95 Hz, 1H), 7.08 (dd, J=2.20, 2.25 Hz, 1H), 6.95-6.90 (m, 1H), 2.32 (t, J=9.65 Hz, 2H), 1.72-1.65 (m, 5H), 1.56-1.46 (m, 4H), 1.06-1.04 (m, 2H). ¹³C NMR (500 MHz, CDCl₃): δ=172.72 (C), 135.46 (C), 133.89 (C), 133.40 (C), 132.60 (C), 129.16 (C), 127.55 (CH), 127.32 (CH), 126.74 (CH), 118.85 (CH), 116.69 (CH), 101.61 (CH), 44.60 (CH), 40.70 (CH2), 39.70 (CH2), 36.87 (CH2), 32.46 (CH2), 31.61 (CH2), 25.16 (CH2). HRMS: (ESI) calculated for C₁₉H₂₂N₃OS⁺ ([M+H]⁺) 340.1478, found 340.1452 M/Z. IRᵥₘₐₓ (cm⁻¹): 2975.62, 1672.22, 1203.33, 721.12. UV (λₘₐₓ nm)=332. HPLC trace: 97.4%.

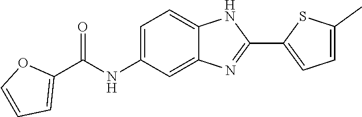 [SMILES: Cc1ccc(-c2nc3cc(NC(=O)c4ccco4)ccc3[nH]2)s1]

N-(2-(5-Methylthiophen-2-yl)-1H-benzo[d]imidazol-5-yl)furan-2-carboxamide (23). Using the general procedures outlined above, 23 was synthesized as a yellow solid in 54% yield (112 mg, 0.346 mmol). ¹H NMR (400 MHz, CDCl₃) δ=8.29 (d, J=2.20 Hz, 1H), 7.88 (d, J=8.96 Hz, 1H), 7.66-7.54 (m, 2H), 7.33 (d, J=4.00 Hz, 1H), 7.03 (t, J=3.52 Hz, 1H), 6.96 (s, 1H), 2.20 (s, 3H). ¹³C NMR (500 MHz, DMSO-d₆): δ=164.54 (C), 156.79 (C), 147.91 (C), 146.30 (C), 143.72 (C), 136.70 (C), 135.49 (C), 130.93 (C), 129.90 (CH), 128.46 (CH), 125.92 (CH), 118.78 (CH), 115.38 (CH), 112.70 (CH), 101.45 (CH), 18.35 (CH₃). HRMS (ESI) calculated for C₁₇H₁₄N₃O₂S⁺ ([M+H]⁺) 324.0801, found 324.0728 M/Z. IRᵥₘₐₓ (cm⁻¹): 2863.25, 2532.10, 1547.44, 709.69. UV (λₘₐₓ nm)=346. HPLC trace: 98.9%.

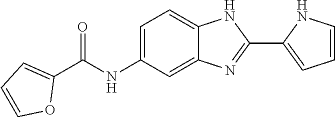 [SMILES: O=C(Nc1ccc2[nH]c(-c3ccc[nH]3)nc2c1)c1ccco1]

N-(2-(1H-Pyrrol-2-yl)-1H-benzo[d]imidazol-5-yl)furan-2-carboxamide (25). Using the general procedures outlined above, 25 was synthesized as a light purple solid in 23% yield (15 mg, 0.051 mmol). ¹H NMR (400 MHz, Methanol-d₄) δ=7.78 (s, 1H), 7.67 (d, J=9.00 Hz, 1H), 7.44 (s, 1H), 7.38 (dd, J=3.75 Hz, 1H), 7.16 (d, J=8.63 Hz, 1H), 6.69 (d, J=3.56 Hz, 1H), 6.78-6.69 (m, 1H), 6.49-6.47 (m, 1H), 6.37-6.36 (m, 1H). ¹³C NMR (500 MHz, DMSO-d₆): δ=164.39 (C), 158.97 (C), 151.32 (C), 148.25 (CH), 143.48 (C), 142.66 (C), 136.61 (C), 134.73 (C), 130.75 (CH), 129.50 (CH), 126.72 (CH), 122.69 (CH), 115.40 (CH), 112.51 (CH), 111.48 (CH), 109.41 (CH). HRMS (ESI) calculated for C₁₆H₁₃N₄O₂⁻ ([M+H]⁺) 293.1033, 293.1010 M/Z. IRᵥₘₐₓ (cm⁻¹): 3080.02, 1660.56, 1182.31, 717.45. UV (λₘₐₓ nm)=328. HPLC trace: 96.6%.

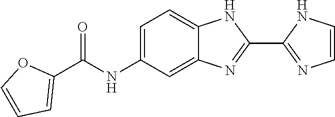 [SMILES: O=C(Nc1ccc2[nH]c(-c3ncc[nH]3)nc2c1)c1ccco1]

N-(2-(1H-Imidazol-2-yl)-1H-benzo[d]imidazol-5-yl)furan-2-carboxamidecarboxamide (26). Using the general procedures outlined above, 26 was synthesized as a beige solid in 6% yield (13 mg, 0.044 mmol). ¹H NMR (400 MHz, Methanol-d₄) δ=8.60 (s, 1H), 8.14 (d, J=2.20 Hz, 1H), 7.93 (t, J=2.20 Hz, 1H), 7.84-7.82 (m, 3H), 7.36 (d, J=4.40 Hz, 1H), 6.71 (q, J=2.10, 2.20 Hz, 1H). ¹³C NMR (500 MHz, DMSO-d₆): δ=168.50 (C), 161.34 (C), 147.88 (C), 146.34 (CH), 139.10 (C), 137.13 (C), 135.51 (C), 126.01 (C), 117.36 (CH), 115.63 (CH), 112.71 (CH), 105.25 (CH). HRMS (ESI) calculated for C₁₅H₂N₅O₂⁺ ([M+H]⁺) 294.0986, found 294.0972 MIZ. IRᵥₘₐₓ (cm⁻¹): 2536.56, 1569.72, 1514.72, 1307.53. UV (λₘₐₓ nm)=338. HPLC trace: 98.1%.

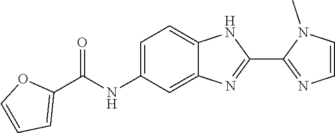 [SMILES: Cn1ccnc1-c1nc2cc(NC(=O)c3ccco3)ccc2[nH]1]

N-(2-(1-Methyl-1H-imidazol-2-yl)-1H-benzo[d]imidazol-5-yl)furan-2-carboxamide (27). Using the general procedures outlined above, 27 was synthesized as a white solid in 16% yield (36 mg, 0.120 mmol). ¹H NMR (400 MHz, Methanol-d₄) δ=7.67 (s, 1H), 7.62 (s, 1H), 7.45 (d, J=1.25 Hz, 1H), 7.21 (d, J=1.75 Hz, 1H), 7.11 (d, J=3.56 Hz, 1H), 6.58 (d, J=4.45 Hz, 1H), 6.50 (d, J=4.20 Hz, 2H), 4.11 (s, 1H). ¹³C NMR (500 MHz, DMSO-d₆): δ=161.39 (C), 156.82 (C), 147.88 (C), 146.34 (C), 144.55 (CH), 142.26 (C), 138.10 (C), 135.51 (C), 126.45 (CH), 122.71 (CH), 121.46 (CH), 115.84 (CH), 112.95 (CH), 110.23 (CH), 36.26 (CH₃). HRMS (ESI) calculated for C₁₆H₁₄N₅O₂⁺ ([M+H]⁺) 309.1142, found 309.1122 M/Z. IRᵥₘₐₓ (cm⁻¹): 2626.77, 1653.16, 1470.28, 759.60. UV (λₘₐₓ nm)=332. HPLC trace: 97.5%.

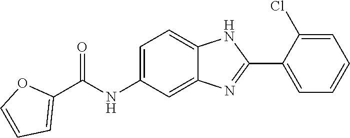 [SMILES: O=C(Nc1ccc2[nH]c(-c3ccccc3Cl)nc2c1)c1ccco1]

N-(2-(2-Chlorophenyl)-1H-benzo[d]imidazol-5-yl)furan-2-carboxamide (29). Using the general procedures outlined above, 29 was synthesized as a white solid in 64% yield (205 mg, 0.54 mmol). ¹H NMR (400 MHz, DMSO-d₆) δ=8.36 (s, 1H), 8.22-8.21 (m, 2H), 8.11 (d, J=4.50 Hz, 1H), 7.91 (d, J=6.20 Hz, 1H), 7.77-7.74 (m, 4H), 7.26 (t, J=4.80 Hz, 1H). ¹³C NMR (500 MHz, DMSO-d₆): δ=160.35 (C), 158.99 (C), 150.11 (C), 139.87 (C), 137.33 (C), 135.207 (C), 133.67 (C), 133.79 (C), 132.98 (C), 132.79 (C), 130.1033 (CH), 129.03 (CH), 128.71 (CH), 128.33 (CH), 119.28 (CH), 115.60 (CH), 108.00 (CH). HRMS (ESI) calculated for C₁₈H₁₃BrN₃O₂⁺ ([M+H]⁺), 382.0113, found, 382.0691 M/Z. IRᵥₘₐₓ (cm⁻¹): 2557.21, 1775.56, 1583.12, 1208.72. UV (λₘₐₓ nm)=332. HPLC trace: 95.3%.

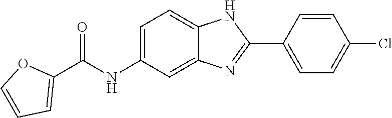 [SMILES: O=C(Nc1ccc2[nH]c(-c3ccc(Cl)cc3)nc2c1)c1ccco1]

N-(2-(4-Chlorophenyl)-1H-benzo[d]imidazol-5-yl)furan-2-carboxamide (30). Using the general procedures outlined above, 30 was synthesized as a white solid in 45% yield (37 mg, 0.097 mmol). ¹H NMR (400 MHz, Methanol-d₄) δ=8.57 (s, 1H), 8.13 (d, J=8.65 Hz, 2H), 7.99 (d, J=4.70 Hz, 1H), 7.81-7.77 (m, 5H), 7.25 (t, J=6.10 Hz, 1H). ¹³C NMR (500 MHz, DMSO-d₆): δ=167.62 (C), 161.73 (C), 133.71 (C), 133.05 (C), 131.83 (C), 131.02 (C), 129.68 (C), 129.29 (CH), 128.50 (CH), 128.00 (CH), 127.78 (CH), 127.53 (CH), 127.24 (CH), 126.50 (CH), 119.29 (CH), 112.60 (CH), 103.76 (CH). FIRMS (ESI) calculated for C₁₈H₁₃BrN₃O₂⁺ ([M+H]⁺), 381.0113, found, 382.0667 M/Z. IRᵥₘₐₓ (cm⁻¹): 2637.94, 1628.27, 1470.53, 1198.46. UV (λₘₐₓ nm)=338. HPLC trace: 97.7%.

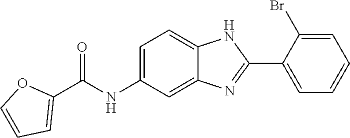 [SMILES: O=C(Nc1ccc2[nH]c(-c3ccccc3Br)nc2c1)c1ccco1]

N-(2-(2-Bromophenyl)-1H-benzo[d]imidazol-5-yl)furan-2-carboxamide (31). Using the general procedures outlined above, 31 was synthesized as a white solid in 64% yield (205 mg, 0.54 mmol). ¹H NMR (400 MHz, DMSO-d₆) δ=8.44 (s, 1H), 8.29-8.28 (m, 1H), 8.23 (dd, J=1.5, 4.7 Hz, 1H), 8.17 (d, J=3.7 Hz, 1H), 8.11 (dd, J=1.5, 3.3 Hz, 1H), 7.92-7.83 (m, 2H), 7.73-7.72 (m, 1H), 7.357 (t, J=4.90 Hz, 1H), 7.26 (t, J=4.80 Hz, 1H), 7.19 (t, J=4.65 Hz, 1H). ¹³C NMR (500 MHz, DMSO-d₆): δ=166.50 (C), 162.04 (C), 142.99 (C), 134.95 (C), 134.07 (C), 132.65 (C), 132.27 (CH), 130.90 (CH), 129.85 (C), 129.50 (CH), 128.21 (CH), 128.01 (CH), 127.57 (CH), 127.13 (CH), 125.26 (CH), 120.58 (CH), 114.00 (CH), 109.88 (CH). HRMS (ESI) calculated for C₁₈H₁₃BrN₃O₂⁺ ([M+H]⁺), 382.0113, found, 382.0186 M/Z. IRᵥₘₐₓ (cm⁻¹): 3318.86, 2655.38, 1675.23, 1259.26. UV (λₘₐₓ nm)=332. HPLC trace: 96.4%.

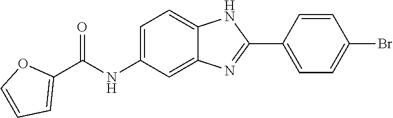 [SMILES: O=C(Nc1ccc2[nH]c(-c3ccc(Br)cc3)nc2c1)c1ccco1]

N-(2-(4-Bromophenyl)-1H-benzo[d]imidazol-5-yl)furan-2-carboxamide (32). Using the general procedures outlined above, 32 was synthesized as a white solid in 45% yield (37 mg, 0.097 mmol). ¹H NMR (400 MHz, DMSO-d₆) δ=8.13 (d, J=8.6 Hz, 1H), 7.81 (s, 1H) 7.79 (m, 5H), 7.36 (d, J=4.35 Hz, 1H), 6.71 (dd, J=2.15, 2.20 Hz, 1H). ¹³C NMR (500 MHz, DMSO-d₆): δ=162.76 (C), 160.77 (C), 150.55 (C), 140.16 (C), 137.01 (C), 135.20 (C), 134.02 (C), 133.31 (C), 133.00 (CH), 132.97 (C), 130.00 (CH), 128.66 (CH), 128.52 (CH), 128.10 (CH), 119.36 (CH), 115.00 (CH). HRMS (ESI) calculated for C₁₈H₁₃BrN₃O₂⁺ ([M+H]⁺), 382.0113, found, 382.0216 M/Z. IRᵥₘₐₓ (cm⁻¹): 3256.76, 2645.28, 1627, 1246.66. UV (λₘₐₓ nm)=336. HPLC trace: 95.7%.

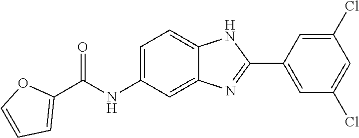 [SMILES: O=C(Nc1ccc2[nH]c(-c3cc(Cl)cc(Cl)c3)nc2c1)c1ccco1]

N-(2-(3,5-Dichlorophenyl)-1H-benzo[d]imidazol-5-yl)furan-2-carboxamide (33). Using the general procedures outlined above, 33 was synthesized as a beige solid in 33% yield (20 mg, 0.053 mmol). ¹H NMR (400 MHz, Methanol-d₄) δ=8.28 (s, 1H), 8.00 (s, 2H), 7.68-7.57 (m, 4H), 7.22 (d, J=4.35 Hz, 1H), 6.58 (q, J=2.20, 2.10 Hz, 1H). ¹³C NMR (500 MHz, DMSO-d₆): δ=162.01 (C), 153.36 (C), 147.81 (C), 143.01 (C), 140.28 (C), 138.69 (C), 137.65 (C), 135.59 (CH), 128.16 (C), 127.50 (CH), 126.75 (CH), 126.09 (CH), 125.62 (CH), 119.13 (CH), 118.32 (CH), 114.90 (CH). HRMS (ESI) calculated for ([M+H]⁺), C₁₈H₁₂Cl₂N₃O₂, 372.0228, found, 372.1550 M/Z. IRᵥₘₐₓ (cm⁻¹): 2645.88, 1627.40, 1517.07, 1255.48. UV (λₘₐₓ nm)=328. HPLC trace: 99.1%.

### Biological Evaluation

### Compound Overview

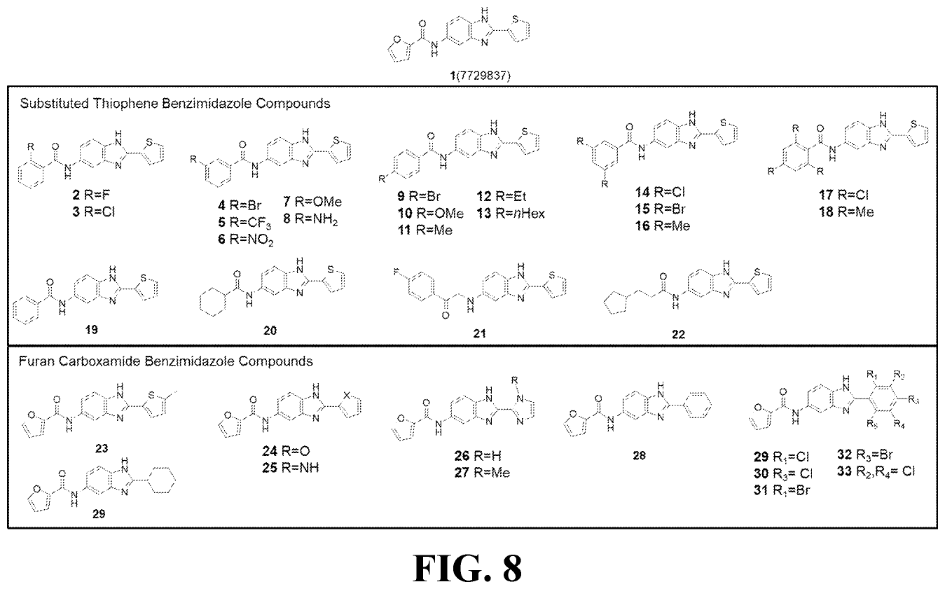

Previously, the 7729837 compounds scaffold was identified. Derivatives were synthesized based upon the 7729837-compound scaffold. Henceforth, compound 7729837 will be referred to as compound 1 (7729837). The synthesized compounds included a variety of derivatives with mono (2-13), di (14-16) or tri (17-18) substituted phenyl groups were evaluated as well as phenyl (19), cyclohexyl (20), 4-fluorophenyl (21) and ethylcyclopentyl (22) (FIG. 8). Several R groups were employed on substituted phenyl derivatives including fluoro (2), chloro (3, 15 and 17), bromo (4, 9, and 15), trichloromethyl (5), nitro (6), methoxy (7, and 10), amino (8), methyl (11, 16 and 18), ethyl (12) and hexyl (13). Next, the impact that varying the identity of the thiophene head group had on activity was studied by synthesizing compounds 23-34 (FIG. 8). Several alterations were made here such as the addition of a two-methyl group on the thiophene (23), the sulfur was replaced with an oxygen (24) or nitrogen (25), the employment of imidazole (26) or methyl imidazole (27) as well as several benzyl derivatives (28-33) and a cyclohexyl group (34). On benzyl derivatives, either chlorine (29, 30, 33) or bromine (31 and 32) was employed (FIG. 8).

### PBP4 Binding of Substituted Thiophene Benzimidazoles

To assess activity, binding affinity for recombinant PBP4 and PBP2a was determined via a Bocillin™ fluorescence assay (Table 14).

TABLE 14
PBP4 binding of compounds 1 (7729837)-22 at 50 μM.

| | Percentage Bocillin ™ |
|---|---|
| Compound | Displacement (%) |
| 1 | 25.66 ± 18.09 |
| (7729837) | |
| 2 | 69.1 ± 28.3 |
| 3 | 129 ± 23.1 |
| 4 | 150.8 ± 11.6 |
| 5 | 87.2 ± 35.6 |
| 6 | 82.1 ± 18.9 |
| 7 | 87.4 ± 27.8 |
| 8 | 134 ± 53.3 |
| 9 | 116.2 ± 5.4 |
| 10 | 103 ± 44.6 |
| 11 | 93.4 ± 33.8 |
| 12 | 108 ± 38.2 |
| 13 | 30.2 ± 15.7 |
| 14 | 107.2 ± 33.8 |
| 15 | <1.0 |
| 16 | 136 ± 10.7 |
| 17 | 61.74 ± 25. |
| 18 | <1.0 |
| 19 | 56.8 ± 37.3 |
| 20 | 127 ± 31.6 |
| 21 | 156 ± 17.1 |
| 22 | 47.1 ± 26.1 |

Bocillin™-FL is a fluorescent penicillin used as a labeling reagent for the detection of PBPs, use of this reagent allows for the measurement derivatives ability to bind to PBP4, thus preventing the binding of Bocillin™-FL which is expressed as percentage inhibition of Bocillin™-FL binding. All compounds were tested at 50 μM. Data in Table 14, exhibited that 1 (7729837) displayed weak Bocillin™-FL inhibition, 25.66%±18.09 towards PBP4. Of the thiophene benzimidazoles, thirteen of the eighteen derivatives showed increased binding affinity for recombinant PBP4.

Derivatives substituted in the 2-position contained either a fluoro (2) or chloro (3) substituent, with compound 3 exhibiting greater PBP4 binding compared to 1 (7729837) (Table 14). Substituents placed in the meta-position included bromo (4), trifluoromethyl (5), nitro (6), methoxy (7) and amino (8). All compounds with substitution in the meta-position were more potent than parent 1 (7729837), with compound 4 (150.8±11.6) being the most active.

Compounds substituted in the para-position possessed a bromo (9), methoxy (10), methyl (11), ethyl (12), or hexyl (13) group. Of these five derivatives, all but compound 13 (30.2±15.7) were more active than 1 (7729837), suggesting that a long, alkyl tail is detrimental for binding affinity. Di-substituted compounds with substituents in the 3-, 5-meta positions included chloro (14), bromo (15) and methyl (16) groups. Compounds 14 (107.2±33.8) and 16 (136±10.7) were more potent compared to 1 (7729837). Two 2-, 4-, 6-substituted derivatives were also evaluated. These derivatives contained either chloro (17) or methyl (18) groups. Neither of these compounds were more active than 1(7729837) in binding PBP4. Compound 19 lacked substitution around the benzyl amide ring and compound 20 possessed a cyclohexyl group while compounds 21 and 22 include additional carbon(s) in the amide link through groups 4-fluorophenyl and ethylcyclopentyl groups, respectively. Compounds 20 (127±31.6) and 21 (156±17.1) were more active than compound 1 (7729837); however, compounds 19 and 22 were not (Table 14). There was not a strong correlation between functional groups and activity; although, derivatives substituted in the para-position (compounds 9-13) were more likely to demonstrate an increase in PBP4 binding.

Potentiation of Substituted Thiophene Benzimidazoles with Oxacillin

To adjuvant activity, each compound was assessed for standalone toxicity with a standard MIC screen. All compounds which registered MICs ≥200 μM were screened as adjuvants at 60 μM. Compounds with an MIC of <200 μM were evaluated at 30% their MIC. Compound 18 was evaluated at 30 μM against all strains and compound 19 was evaluated at 30 μM against AH-1263 and ATCC BAA-1556 and at 15 μM against AH-2204. Compounds were then evaluated for their activity in potentiating oxacillin. Activity is defined here by the ability to lower the MIC by four-fold or greater. Compound 1 (7729837) reduced the MIC of oxacillin by 16-fold, 16-fold, and 64-fold against strains AH-1263, ATCC BAA-1556 and AH-2204, respectively (Table 15).

TABLE 15
Potentiation activity of oxacillin with thiophene
benzimidazole compounds against MRSA strains AH-1263,
ATCC BAA-1556, and AH-2204. All compounds were used
at 60 μM except for compounds 18 and 19. Both
compounds 18 and 19 were evaluated at 30 μM.
Oxacillin MIC (μg/mL) (fold reduction)

| | | ATCC | |
|---|---|---|---|
| Compounds | AH-1263 | BAA-1556 | AH-2204 |
| — | 16 | 32 | 32 |
| 1 | 1 (16) | 2 (16) | 0.5 (64) |
| (7729837) | | | |
| 2 | 32 (—) | 32 (—) | 16 (2) |
| 3 | 8 (2) | 8 (4) | 4 (8) |
| 4 | 0.5 (32) | 0.25 (128) | 0.125 (256) |
| 5 | 16 (—) | 16 (2) | 4 (8) |
| 6 | 8 (2) | 4 (8) | 2 (16) |
| 7 | 16 (—) | 16 (2) | 8 (4) |
| 8 | 4 (4) | 0.5 (64) | 0.5 (64) |
| 9 | 16 (—) | 16 (2) | 16 (2) |
| 10 | 2 (8) | 2 (16) | 2 (16) |
| 11 | 8 (2) | 32 (—) | 2 (16) |
| 12 | 32 (—) | 32 (—) | 2 (16) |
| 13 | 4 (4) | 8 (4) | 4 (8) |
| 14 | 4 (4) | 1 (32) | 1 (32) |
| 15 | 4 (4) | 2 (16) | 4 (8) |
| 16 | 2 (8) | 1 (32) | 0.5 (64) |
| 17 | 4 (4) | 4 (8) | 1 (32) |
| 18 | 0.5 (32) | 1 (32) | 2 (16) |
| 19 | 8 (2) | 4 (8) | 4 (8) |
| 20 | 8 (2) | 8 (2) | 2 (16) |
| 21 | 8 (2) | 16 (2) | 8 (4) |
| 22 | 16 (—) | 16 (2) | 4 (8) |
| 23 | 16 (—) | 32 (—) | 32 (—) |

Derivatives substituted in the ortho-position contained either a fluoro (compound 2) or chloro (compound 3) substituent. Compound 2 was inactive while compound 3 reduced the MIC of oxacillin by four-fold and 8-fold against ATCC BAA-1556 and AH-2204, respectively. Substituents that were placed in the meta-position include bromo (compound 4), trifluoromethyl (compound 5), nitro (compound 6), methoxy (compound 7), and amino (compound 8). All meta-substituted derivatives reduced the MIC of oxacillin by at least four-fold towards at least one MRSA strain with compound 4 being the most active meta-substituted derivative, showing a 32-fold, 128-fold and 256-fold decrease in MIC against AH-1263, ATCC BAA-1556 and AH-2204, respectively (Table 15). Compounds substituted in the para-position possessed a bromo (compound 9), methoxy (compound 10), methyl (compound 11), ethyl (compound 12), or hexyl (compound 13) group. Compound 9 was inactive while compounds 11 and 12 decreased the MIC of oxacillin by 16-fold against AH-2204. Compounds 13 and 14 dropped the MIC by at least four-fold against all strains, with 13 being the most potent dropping the MIC by 32-fold against ATCC BAA-1556 and AH-2204.

Di-substituted compounds with substituents in the 3-, 5-positions included chloro (compound 14), bromo (compound 15), and methyl (compound 16) groups. These compounds all demonstrated a decrease in MIC by four-fold or greater against all strains with compound 16 being the most potent, lowering the MIC 64-fold against AH-2204. Two 2-, 4-, 6-substituted derivatives were also evaluated, containing either chloro (compound 17) or methyl (compound 18) groups. Compound 18 reduced the MIC of oxacillin by 32-fold, 32-fold, and 16-fold against AH-1263, ATCC BAA-1556 and AH-2204, respectively whereas compound 19 reduced the MIC by 8-fold against ATCC BAA-1556 and AH-2204 and was of negligible activity against AH-1264 (Table 15). Compound 19 lacked substitution around the benzyl amide ring and was able to drop the MIC 16-fold against AH-2204 but was otherwise inactive. Additionally, compound 20, which contained a cyclohexyl group, decreased the MIC by four-fold towards AH-2204, but exhibited negligible activity towards other strains. Compounds 21 and 22, which include additional carbon(s) in the amide link through groups 4-fluorophenyl and ethylcyclopentyl groups, respectively, with compound 22 lowering the MIC 8-fold against AH-2204 and compound 21 showing no activity.

While several compounds lowered the MIC by at least four-fold, the only derivatives with greater activity than 1 (7729837) were compounds 4 and 8 (Table 15). Compound 4 decreased the MIC 8-fold compared to compound 1 (7729837) against ATCC BAA-1556 and AH-2204 and decreased the MIC two-fold against AH-1263, which is considered to be equipotent. Compound 8 demonstrated a four-fold decrease in MIC compared to compound 1 (7729837) against ATCC BAA-1556, was equipotent against AH-2204 and less potent against AH-2204. Additional compounds that were equipotent to compound 1 (7729837) against at least one strain including compounds 14, 16, 17 and 18.

Potentiation of Substituted Thiophene Benzimidazoles with β-Lactams

Following the initial screening, all compounds were evaluated in tandem with two additional β-lactam antibiotics, ampicillin and cefoxitin (Table 16). This was done to determine if additional compounds would exhibit increased activity over compound 1 (7729837) with different β-lactams. Compound 1 (7729837) was inactive when used with ampicillin towards all strains and active only against AH-1263 when combined with cefoxitin, dropping the MIC by four-fold. Of the 21 derivatives in thiophene benzimidazole compounds, 16 were more active than compound 1 (7729837) against at least one strain when used in tandem with ampicillin and 8 were more potent in tandem with cefoxitin.

Compounds 4, 6, 8, 16, 18 and 19, were more potent compared to compound 1 (7729837) against all strains (Table 16). Compound 4, which was the most potent derivative against AH-2204, increased potency compared to compound 1 (7729837) by 8-fold, 16-fold, and 32-fold against AH-1263, ATCC BAA-1556 and AH-2204, respectively. Compound 6 was the most potent derivative towards AH-1263, decreasing the MIC by 64-fold, 32-fold and 4-fold compared to 1 (7729837) against AH-1263, ATCC BAA-1556 and AH-2204, respectively. Compound 8 dropped the ampicillin MIC by 8-fold, 16-fold and 8-fold compared to 1 (7729837). Compound 16 increased potency by 8-fold against all strains whereas compound 18 increased potency by 8-fold, 4-fold, and 8-fold, respectively. Lastly, compound 19 increased potency by 8-fold, 8-fold, and 4-fold against AH-1263, ATCC BAA-1556 and AH-2204, respectively.

TABLE 16
Potentiation of β-lactams with substituted thiophene benzimidazoles against MRSA
strains AH-1263, ATCC BAA-1556, and AH-2204. All compounds were used at 60 μM except
for compounds 18 and 19. Both compounds 18 and 19 were evaluated at 30 μM.

| | MIC (μg/mL) (fold reduction) | | | | | | | | | | | |
|---|---|---|---|---|---|---|---|---|---|---|---|---|
| | Ampicillin | | | | | | Cefotaxime | | | | | |
| | AH- | | ATCC | | AH- | | AH- | | ATCC | | AH- | |
| Compound | 1263 | | BAA-1556 | | 2204 | | 1263 | | BAA-1556 | | 2204 | |
| — | 16 | | 16 | | 2 | | 32 | | 32 | | 16 | |
| 1 (7729837) | 8 | (2) | 8 | (2) | 2 | (—) | 8 | (4) | 16 | (2) | 8 | (2) |
| 2 | 4 | (4) | 8 | (2) | 4 | (—) | 32 | (—) | 32 | (—) | 16 | (—) |
| 3 | 1 | (16) | 16 | (—) | 2 | (—) | 4 | (8) | 8 | (4) | 4 | (4) |
| 4 | 1 | (16) | 0.5 | (32) | 0.0623 | (32) | 1 | (32) | 0.5 | (64) | 0.5 | (32) |
| 5 | 2 | (8) | 8 | (2) | 2 | (—) | 16 | (2) | 16 | (2) | 8 | (2) |
| 6 | 0.25 | (64) | 1 | (16) | 0.5 | (4) | 2 | (16) | 1 | (32) | 4 | (4) |
| 7 | 4 | (4) | 8 | (2) | 2 | (—) | 16 | (2) | 32 | (—) | 16 | (—) |
| 8 | 1 | (16) | 0.5 | (32) | 0.25 | (8) | 8 | (4) | 4 | (8) | 4 | (4) |
| 9 | 8 | (2) | 4 | (4) | 4 | (—) | 16 | (2) | 16 | (2) | 16 | (—) |
| 10 | 2 | (8) | 2 | (8) | 2 | (—) | 2 | (16) | 1 | 932) | 2 | (8) |
| 11 | 2 | (8) | 16 | (—) | 2 | (—) | 32 | (—) | 32 | (—) | 16 | (—) |
| 12 | 0.5 | (32) | 8 | (2) | 0.125 | (16) | 8 | (4) | 16 | (2) | 4 | (4) |
| 13 | 4 | (4) | 32 | (—) | 1 | (2) | 8 | (4) | 16 | (2) | 8 | (2) |
| 14 | 0.5 | (32) | 8 | (2) | 2 | (—) | 32 | (—) | 32 | (—) | 16 | (—) |
| 15 | 2 | (8) | 8 | (2) | 1 | (2) | 8 | (4) | 4 | (8) | 8 | (2) |
| 16 | 2 | (8) | 2 | (8) | 0.25 | (8) | 16 | (2) | 16 | (2) | 16 | (—) |
| 17 | 1 | (16) | 2 | (8) | 1 | (2) | 4 | (8) | 4 | (8) | 2 | (8) |
| 18 | 1 | (16) | 2 | (8) | 0.25 | (8) | 0.5 | (64) | 1 | (32) | 0.5 | (32) |
| 19 | 1 | (16) | 1 | (16) | 0.5 | (4) | 4 | (8) | 4 | (8) | 8 | (2) |
| 20 | 4 | (—) | 4 | (—) | 2 | (—) | 8 | (4) | 16 | (2) | 8 | (2) |
| 21 | 2 | (8) | 16 | (—) | 2 | (—) | 32 | (—) | 16 | (2) | 8 | (2) |
| 22 | 2 | (8) | 16 | (—) | 2 | (—) | 8 | (4) | 16 | (2) | 8 | (2) |
| 23 | 0.5 | (32) | 8 | (2) | 1 | (2) | 8 | (4) | 16 | (2) | 8 | (2) |

When used in tandem with cefoxitin, compounds 4 and 18 dropped the MIC at least four-fold towards all strains compared to compound 1 (7729837). Compound 4 dropped the MIC by 8-fold, 32-fold and 16-fold compared to compound 1 (7729837) against AH-1263, ATCC BAA-1556 and AH-2204, respectively. Compound 18 lowered the MIC by 16-fold against all strains compared to compound 1 (7729837). Compounds 6, 8, 15, 17, 19 also exhibited greater potency than compound 1 (7729837) against one or two strains. The unsubstituted phenyl (compound 20) was again less active than the majority of substituted derivatives, active only with cefoxitin against AH-1263, with a four-fold MIC reduction.

### PBP4 Binding of Substituted Furan Carboxamide Benzimidazoles

In in the carboxamide benzimidazole compounds, five of the twelve derivatives exhibited binding to PBP4 to a greater degree than compound 1 (7729837) (Table 17). Compounds 24 (108±25.13), 25 (110±41.9), 26 (105±14.1), 29 (111.2±26.1) and 30 (115±26.6) demonstrated an increase in binding. Compounds 24, 25 and 26 contain either oxygen or nitrogen in place of sulfur and exhibit binding to PBP4. Compound 27, which has an added methyl group showed <1.0% inhibition of Bocillin™. Therefore, compounds with a five membered ring were typically more active than those with a six membered ring.

TABLE 17
PBP4 binding of substituted furan carboxamide
benzimidazoles at 50 μM.

| | Bocillin ™ |
|---|---|
| Compound | Displacement (%) |
| 1 | 25.66 ± 18.09 |
| (7729837) | |
| 23 | 46.8 ± 32.8 |
| 24 | 108 ± 25.13 |
| 25 | 110 ± 41.9 |
| 26 | 105 ± 14.1 |
| 27 | <1.0 |
| 28 | <1.0 |
| 29 | 111.2 ± 26.1 |
| 30 | 115 ± 26.6 |
| 31 | 34.2 ± 38.2 |
| 32 | 45.7 ± 18.7 |
| 33 | 56.5 ± 35.6 |
| 34 | 60.3 ± 43.8 |

Potentiation of Furan Carboxamide Benzimidazoles with Oxacillin and β-Lactams

Furan benzimidazole derivatives (compounds 23-34) were evaluated for adjuvant activity in tandem with oxacillin, ampicillin and cefoxitin. Compounds 23, 24, 25, 26, 27, 32, 33 and 34 reduced the oxacillin MIC by four-fold against at least one MRSA strain (Table 18). Compound 28 was the most potent derivative in the towards ATCC BAA-1556 and AH-2204, lowering oxacillin's MIC by four-fold and 64-fold respectively. Compound 25 was the most potent derivative against AH-1263 with oxacillin, lower the MIC by 8-fold. There were no compounds able to lower the oxacillin MIC to a greater degree than 1 (7729837), although compound 28 was equipotent against AH-2204. The furan carboxamide benzimidazole compounds were also then evaluated in tandem with ampicillin and cefoxitin.

TABLE 18
Potentiation of oxacillin and β-Lactams with furan carboxamide compounds. All compounds were evaluated at 60 μM.

| | MIC (μg/mL) (fold reduction) | | | | | | | | | | | | | | | | | |
|---|---|---|---|---|---|---|---|---|---|---|---|---|---|---|---|---|---|---|
| | Oxacillin | | | | | | Ampicillin | | | | | | Cefotaxime | | | | | |
| | AH- | | ATCC | | AH- | | AH- | | ATCC | | AH- | | AH- | | ATCC | | AH- | |
| Compound | 1263 | | BAA-1556 | | 2204 | | 1263 | | BAA-1556 | | 2204 | | 1263 | | BAA-1556 | | 2204 | |
| — | 16 | | 32 | | 32 | | 16 | | 16 | | 2 | | 32 | | 32 | | 16 | |
| 1 (7729837) | 1 | (16) | 2 | (16) | 0.6 | (64) | 8 | (2) | 8 | (2) | 2 | (—) | 8 | (4) | 16 | (2) | 8 | (2) |
| 23 | 16 | (—) | 32 | (—) | 8 | (4) | 2 | (8) | 2 | (8) | 4 | (—) | 32 | (—) | 16 | (2) | 16 | (—) |
| 24 | 2 | (8) | 16 | (2) | 8 | (4) | 8 | (2) | 16 | (—) | 4 | (—) | 16 | (2) | 32 | (—) | 16 | (—) |
| 25 | 4 | (4) | 8 | (4) | 1 | (32) | 16 | (—) | 8 | (2) | 0.5 | (4) | 32 | (—) | 32 | (—) | 8 | (2) |
| 26 | 16 | (—) | 32 | (—) | 8 | (4) | 2 | (8) | 8 | (2) | 2 | (—) | 32 | (—) | 16 | (2) | 16 | (—) |
| 27 | 8 | (2) | 8 | (4) | 0.5 | (64) | 2 | (8) | 0.5 | (32) | 0.25 | (4) | 32 | (—) | 32 | (—) | 16 | (—) |
| 28 | 8 | (2) | 16 | (2) | 16 | (2) | 2 | (8) | 16 | (—) | 2 | (—) | 32 | (—) | 16 | (2) | 16 | (—) |
| 29 | 16 | (—) | 16 | (—) | 16 | (—) | 16 | (—) | 16 | (—) | 2 | (—) | 16 | (2) | 16 | (2) | 16 | (—) |
| 30 | 16 | (—) | 16 | (—) | 16 | (2) | 16 | (—) | 16 | (—) | 4 | (—) | 16 | (2) | 16 | (2) | 16 | (—) |
| 31 | 16 | (—) | 32 | (—) | 16 | (2) | 8 | (2) | 4 | (4) | 4 | (—) | 32 | (—) | 32 | (—) | 16 | (—) |
| 32 | 8 | (2) | 16 | (2) | 4 | (8) | 4 | (4) | 4 | (4) | 4 | (—) | 16 | (2) | 16 | (2) | 8 | (2) |
| 33 | 16 | (—) | 16 | (2) | 4 | (8) | 1 | (16) | 16 | (—) | 2 | (—) | 32 | (—) | 16 | (2) | 16 | (—) |
| 34 | 16 | (—) | 32 | (—) | 8 | (4) | 2 | (8) | 16 | (—) | 2 | (—) | 32 | (—) | 32 | (—) | 8 | (2) |

When employed with ampicillin, seven of the twelve furan carboxamide benzimidazole derivatives were able to lower the MIC by four-fold or greater. Additionally, compounds 23, 25, 26, 27, 28, 33, and 34 were more potent than 1 (7729837) against at least one MRSA strain (Table 18). Compound 27 was the only compound more potent than 1 (7729837) against all strains, dropping the MIC by four-fold, 16-fold, and fourfold greater against AH-1263, ATCC BAA-1556 and AH-2204, respectively. When used in tandem with cefoxitin, the furan carboxamide benzimidazoles were unable to lower the MIC by four-fold or greater.

Potentiation of Top Compounds with Penicillin G, Piperacillin, Amoxicillin, and Cephalothin

Five derivatives (4, 6, 8, 18 and 28) were selected and evaluated for activity with additional β-lactam antibiotics, including penicillin G, piperacillin, amoxicillin and cephalothin. Compounds 4 and 8 was selected because they were able to potentiate oxacillin, ampicillin and cefoxitin to a greater degree than the parent compound, 1 (7729837), against multiple strains (Table 18). Compounds 6 and 18 were selected for their potency when used in combination with ampicillin and cefoxitin (Table 16). Compound 28 was selected because it was the most potent derivative in furan benzimidazoles (Table 18). Derivatives 4, 8 and 6 also showed a greater binding affinity towards PBP4 compared to compound 1 (7729837) (Table 14). Tables 19A-19D, below, show potentiation of penicillin G, piperacillin, amoxicillin, and cephalothin with select PBP4 compounds against MRSA strains AH-1263, ATCC BAA-1556, and AH-2204.

TABLE 19A
Potentiation of penicillin G with select PBP4 compounds
against MRSA strains AH-1263, ATCC BAA-1556, and AH-2204.

MIC (μg/mL) (fold reduction)
Penicillin G

| | | ATCC | |
|---|---|---|---|
| Compound | AH-1263 | BAA-1556 | AH-2204 |
| — | 8 | 2 | 1 |
| 1 | 4(2) | 2(—) | 1(—) |
| (7729837) | | | |
| 4 | 1(8) | 2(—) | 0.5(2) |
| 6 | 1(8) | 2(—) | 1(—) |
| 8 | 0.25 (32) | 0.5(4) | 0.125 (8) |
| 18 | 1(8) | 2(—) | 1(—) |
| 28 | 4(2) | 2(—) | 1(—) |

TABLE 19B
Potentiation of piperacillin with select PBP4 compounds
against MRSA strains AH-1263, ATCC BAA-1556, and AH-2204.

MIC (μg/mL) (fold reduction)
Piperacillin

| | | ATCC | |
|---|---|---|---|
| Compound | AH-1263 | BAA-1556 | AH-2204 |
| — | 16 | 16 | 4 |
| 1 | 8(2) | 8(2) | 4(—) |
| (7729837) | | | |
| 4 | 8(2) | 8(2) | 1(4) |
| 6 | 16(—) | 16(—) | 2(2) |
| 8 | 16(—) | 8(2) | 0.25 (16) |
| 18 | 8(2) | 16(—) | 2(2) |
| 28 | 16(—) | 16(—) | 1(4) |

TABLE 19C
Potentiation of amoxicillin with select PBP4 compounds against
MRSA strains AH-1263, ATCC BAA-1556, and AH-2204.

MIC (μg/mL) (fold reduction)
Amoxicillin

| | | ATCC | |
|---|---|---|---|
| Compound | AH-1263 | BAA-1556 | AH-2204 |
| — | 8 | 8 | 2 |
| 1 | 8(—) | 8(—) | 2(—) |
| (7729837) | | | |
| 4 | 2(4) | 1(8) | 1(2) |
| 6 | 4(2) | 8(—) | 1(2) |
| 8 | 4(2) | 0.125(64) | 0.125(16) |
| 18 | 2(4) | 2(4) | 2(2) |
| 28 | 8(—) | 8(—) | 1(2) |

TABLE 19D
Potentiation of cephalothin with select PBP4 compounds against
MRSA strains AH-1263, ATCC BAA-1556, and AH-2204.

MIC (μg/mL) (fold reduction)
Cephalothin

| | | ATCC | |
|---|---|---|---|
| Compound | AH-1263 | BAA-1556 | AH-2204 |
| — | 32 | 16 | 4 |
| 1 | 1(32) | 1(16) | 0.125(32) |
| (7729837) | | | |
| 4 | 1(32) | 0.5 (32) | 0.062(64) |
| 6 | 2(16) | 2(8) | 0.25 (16) |
| 8 | 2(16) | 0.5(32) | 0.25(16) |
| 18 | 1(32) | 1(16) | 0.125(32) |
| 28 | 16(2) | 8(2) | 0.5(16) |

Compound 1 (7729837) was unable to lower the MIC of penicillin G, piperacillin, or amoxicillin by four-fold or greater (Tables 19A-19C). However, compound 1 (7729837) lowered the MIC of cephalothin by 32-fold, 16-fold, and 32-fold against AH-1263, ATCC BAA-1556 and AH-2204, respectively (Table 19D). Several derivatives more active than compound 1 (7729837) were identified from these studies. When used in tandem with penicillin, four of the five derivatives (compounds 4, 6, 8 and 18) were able to lower the MIC by four-fold or greater against at least one MRSA strain. Compound 8 was most potent, lowering the MIC by 32-fold, 4-fold, and 8-fold against AH-1263, ATCC BAA-1556 and AH-2204, respectively. In tandem with piperacillin, compounds 4 and 8 were able to lower the MIC by four-fold or greater, with compound 4 lowering the MIC four-fold and 8 16-fold against AH-2204. With amoxicillin, three of the five derivatives (compounds 4, 8, and 18) were able to lower the MIC by four-fold or greater. Compound 4 lowered the MIC by four-fold and 8-fold against AH-1263 and ATCC BAA-1556, while 8 lowered the MIC by 64-fold and 16-fold against ATCC BAA-1556 and AH-2204 and compound 18 lowered the MIC by four-fold against AH-1263 and ATCC BAA-1556.

With cephalothin, four of the five derivatives (compounds 4, 6, 8 and 18) lowered the MIC 8-fold or greater towards all strains. Compound 1 (7729837) was more active with cephalothin compared to penicillin G, piperacillin, and amoxicillin so it was expected that derivatives would be as well. Compound 4 was the most potent derivative, dropping the MIC 32-fold against AH-1263 and ATCC BAA-1556 and 64-fold against AH-2204. These values were within two-fold of the MIC of compound 1 (7729837).

Potentiation of Compound 1 (7729837), 4, and 8 with Oxacillin Against MRSA Strains

To further evaluate the potentiation activity of select compounds, derivatives 4 and 8 were evaluated against a panel of MRSA strains for their efficacy in potentiating oxacillin. Compounds 4 and 8 were selected because they were more active than 1 (7729837) in combination with oxacillin and were also able to drop the MICs of each β-lactam evaluated here by four-fold or greater against at least one MRSA strain.

When comparing all 11 strains, compounds 4 and 8 were most active against AH-2204, with 1 (7729837) lowering the MIC 64-fold, 4 by 256-fold and 8 by 64-fold (Table 20). The compounds were most potent towards strain BAA-1770, with 1 (7729837) and 4 dropping the MIC by 32-fold, and 8 lowering the MIC by 64-fold. Against strains BAA-1685, BAA-44 and 700699 all compounds were inactive.

TABLE 20
Minimum inhibitory concentration (MIC) of oxacillin with compounds
1 (7729837), 4, and 8 at 60 μM again various MRSA strains.

| | |
|---|---|
| | Oxacillin MIC (μg/mL) (fold reduction) |
| MRSA | Compounds |

| Strains | 1 | 4 | 8 |
|---|---|---|---|
| AH-1263 | 1(16) | 0.5(32) | 4(4) |
| ATCC | 2(16) | 0.25(128) | 0.5(64) |
| BAA-1556 | | | |
| AH-2204 | 0.5(64) | 0.125(256) | 0.5(64) |
| BAA-811 | 64(4) | 256(—) | 128 (2) |
| 700789 | 464(4) | 128(2) | 64(4) |
| BAA-1770 | 16(32) | 16(32) | 8(64) |
| 43300 | 16(32) | 256(—) | 128(4) |
| BAA-1685 | 64(—) | 64(—) | 64(—) |
| 33591 | 32(4) | 128(—) | 64(2) |
| BAA-44 | 16(—) | 16(—) | 16(—) |
| 700699 | 16(—) | 17(—) | 18(—) |

### Human Cell Cytotoxicity Measures of PBP4 Inhibitors

To determine the toxicity of derivatives towards mammalian cell, a cell viability assay was performed using human liver epithelial cells (HEPG2) cells. Compounds resulting in cell viability above 70% were considered non-cytotoxic. Treatment with compound 1 (7729837) resulted in 95±16.8% cell viability (Table 21).

TABLE 21
Cell viability of compounds 1 (7729837)-22 at 100 μM in
HEPG2 cells. The concentration of HEPG2 cells was approximately
2.5 × 10⁵ cells/mL

| | Toxicity with |
|---|---|
| Compound | HEPG2 Cells |
| 1 | 95 ± 16.8 |
| (7729837) | |
| 2 | 99 ± 7.9 |
| 3 | 93.9 ± 10.5 |
| 4 | 113 ± 1.8 |
| 5 | 91.7 ± 12.6 |
| 6 | 106 ± 6.6 |
| 7 | 72 ± 3.9 |
| 8 | 87.7 ± 10.7 |
| 9 | 102 ± 31 |
| 10 | 77.7 ± 5.7 |
| 11 | 98.9 ± 16.2 |
| 12 | 82.2 ± 20.7 |
| 13 | 112 ± 21.8 |
| 14 | 97.8 ± 16.3 |
| 15 | 71.54 ± 3.6 |
| 16 | 91.7 ± 3.6 |
| 17 | 77.9 ± 7.8 |
| 18 | 92 ± 9.9 |
| 19 | 86.6 ± 6.7 |
| 20 | 75 ± 1.4 |
| 21 | 84.3 ± 3.6 |
| 22 | 132 ± 6.4 |

Of the thiophene benzimidazole compounds, only compounds 7, 12, and 15 demonstrated cytotoxicity. Of the furan benzimidazoles, derivatives demonstrated low to no levels of cytotoxicity, similar to the thiophene benzimidazoles. All compounds with the exception of compounds 28 and 31, showed cell viability of 70% or higher (Table 22).

TABLE 22
Cell viability of compounds 23-34 at 100 μM
in HEPG2 cells. The concentration of HEPG2 cells was
approximately 2.5 × 10⁵ cells/mL.

| | Toxicity with |
|---|---|
| Compound | HEPG2 Cells |
| 1 | 95 ± 16.8 |
| (7729837) | |
| 23 | 103 ± 12.8 |
| 24 | 102 ± 3.8 |
| 25 | 94.9 ± 5.7 |
| 26 | 103 ± 7.9 |
| 27 | 101 ± 23.4 |
| 28 | 73 ± 5.2 |
| 29 | 94 ± 24 |
| 30 | 90.2 ± 10.3 |
| 31 | 81.4 ± 17.6 |
| 32 | 99 ± 20.4 |
| 33 | 151 ± 11.3 |
| 34 | 97 ± 5.8 |

### Additional Evaluation of Compounds

Dose-response activity of compounds 1 (7729837), 4, and 8 with oxacillin was also determined down to a concentration of 10 μM against ATCC BAA-1556.

According to the Clinical and Laboratory Standards Institute, the break point of oxacillin is ≤2 μg/mL. Against strain ATCC BAA-1556, the MIC for oxacillin was determined to be 32 μg/mL. All compounds were inactive under 50 μM, thus compounds were evaluated at 60 μM.

To further quantify potentiation activity, time-kill curves were created for compounds 4 (FIG. 9A) and 8 (FIG. 9B) as representative lead compounds (FIG. 9A-B). Compounds were evaluated in tandem with oxacillin against strain ATCC BAA-1556. Compound with 2 μg/mL oxacillin and 60 μM compound 4 (FIG. 9A) was able to reduce the number of viable cells after 2 hours, although from 4-24 hours the number of viable cells increases. Compound 8 (FIG. 9B) showed decreasing viable cells from hours 2-6, although from hours 6-24 there was an increase in viable cells.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the disclosure are set out in the following numbered clauses:

Clause 1. A pharmaceutical composition comprising a compound of formula (I), or a pharmaceutically acceptable salt thereof, and a pharmaceutically acceptable carrier,

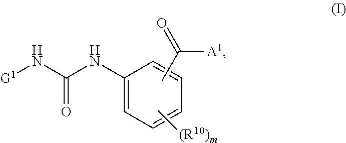

wherein:
A¹ is

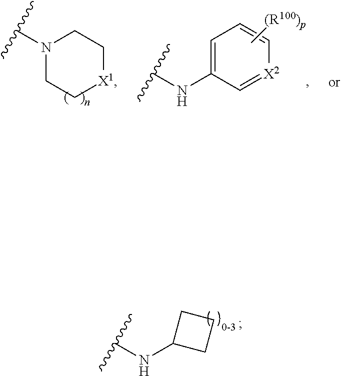

G¹ is a 6- to 12-membered aryl, C₃₋₁₀carbocyclyl, a 5- to 12-membered heteroaryl, or a 4- to 12-membered heterocyclyl, wherein G¹ is optionally substituted with 1-3 R¹;
X¹ is CHR^{X}, C═O, or O;
X² is CH or N;
R^{X}, at each occurrence, is C₁₋₆alkyl, hydrogen, C₁₋₄haloalkyl, halogen, or cyano;
R¹, at each occurrence, is independently halogen, cyano, C₁₋₆alkyl, C₁₋₆haloalkyl, —OR^{1a}, —SR^{1a}, —CO₂R^{1a}, —C(O)R^{1a}, —SO₂R^{1b}, —N(R^{1b})₂, —CO₂N(R^{1b})₂, —NO₂, G^{1a}, —OG^{1a}, —SG^{1a}, —N(R^{1b})-G^{1b}, -L¹-Y¹, —O-L¹-Y¹, —S-L¹-Y¹, or —N(R^{1b})-L¹-Y¹;
L¹, at each occurrence, is independently a C₁₋₆alkylene, wherein optionally 1 or 2 methylene groups in the alkylene of L¹ are independently replaced with —O—, —S—, —SO₂—, —C(O)—, or —N(R^{1b})—, wherein 2 methylene groups replaced with —O—, —S—, —SO₂—, or N(R^{1b})— are separated by two or more carbon atoms in the alkylene;
Y¹, at each occurrence, is independently hydrogen, halogen, cyano, C₁₋₆alkyl, C₁₋₆haloalkyl, —OR^{1a}, —SR^{1a}, —CO₂R^{1a}, —C(O)R^{1a}, —SO₂R^{1b}, —N(R^{1b})₂, —CO₂N(R^{1b})₂, G^{1a}, or —OG^{1a};
R^{1a}, at each occurrence, is independently hydrogen, C₁₋₆alkyl, or C₁₋₂haloalkyl;
R^{1b}, at each occurrence, is independently hydrogen or C₁₋₆alkyl;
G^{1a}, at each occurrence, is independently C₃₋₆carbocyclyl, phenyl, a 4- to 6-membered heterocyclyl, or a 5- to 6-membered heteroaryl, wherein G^{1a} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
G^{1b}, at each occurrence, is independently C₃₋₆carbocyclyl or phenyl, wherein G^{1b} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
R¹⁰, at each occurrence, is independently halogen, C₁₋₄alkyl, —OC₁₋₄alkyl, C₁₋₂haloalkyl, cyano, or —OC₁₋₂haloalkyl;
R¹⁰⁰, at each occurrence, is independently halogen, C₁₋₄alkyl, —OC₁₋₄alkyl, C₁₋₂haloalkyl, cyano, or —OC₁₋₂haloalkyl;
m is 0, 1, 2, 3, or 4;
n is 0, 1, or 2; and
p is 0, 1, 2, or 3.

Clause 2. The pharmaceutical composition of clause 1, wherein the compound of formula (I) is a compound of formula (I-a):

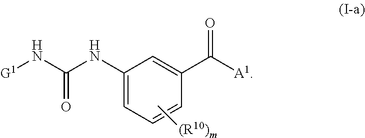

Clause 3. The pharmaceutical composition of clauses 1 or 2, wherein G¹ is the 6- to 12-membered aryl.

Clause 4. The pharmaceutical composition of any one of clauses 1-3, wherein the 6- to 12-membered aryl at G¹ is phenyl.

Clause 5. The pharmaceutical composition of any one of clauses 1-4, wherein G¹ is substituted with 1-2 substituents selected from the group consisting of halogen, C₁₋₆alkyl, —OC₁₋₄alkyl, —NH₂, —OC₁₋₂fluoroalkyl, C₁₋₂fluoroalkyl, or —NO₂.

Clause 6. The pharmaceutical composition of any one of clauses 1-5, wherein G¹ is

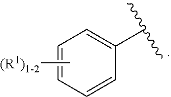

Clause 7. The pharmaceutical composition of any one of clauses 1-6, wherein m is 0.

Clause 8. The pharmaceutical composition of any one of clauses 1-7, wherein A¹ is

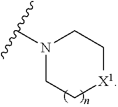

Clause 9. The pharmaceutical composition of any one of clauses 1-8, wherein n is 1.

Clause 10. The pharmaceutical composition of any one of clauses 1-9, wherein X¹ is CHR^{X}.

Clause 11. The pharmaceutical composition of claim 10, wherein R^{X} is C₁₋₆alkyl.

Clause 12. The pharmaceutical composition of claim 11, wherein R^{X} is methyl.

Clause 13. The pharmaceutical composition of any one of clauses 1-7, wherein X¹ is C═O.

Clause 14. The pharmaceutical composition of any one of clauses 1-7, wherein X¹ is O.

Clause 15. The pharmaceutical composition of any one of clauses 1-14, wherein A¹ is

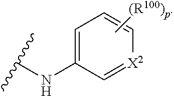

Clause 16. The pharmaceutical composition of any one of clauses 1-15, wherein X² is CH.

Clause 17. The pharmaceutical composition of any one of clauses 1-16, wherein p is 1 or 2.

Clause 18. The pharmaceutical composition of any one of clauses 1-17, wherein R¹⁰⁰ is F, Cl, —CH₃, or —OCH₃.

Clause 19. The pharmaceutical composition of claim 15, wherein X² is N.

Clause 20. The pharmaceutical composition of clause 1, wherein the compound of formula (I) is selected from the group consisting of:

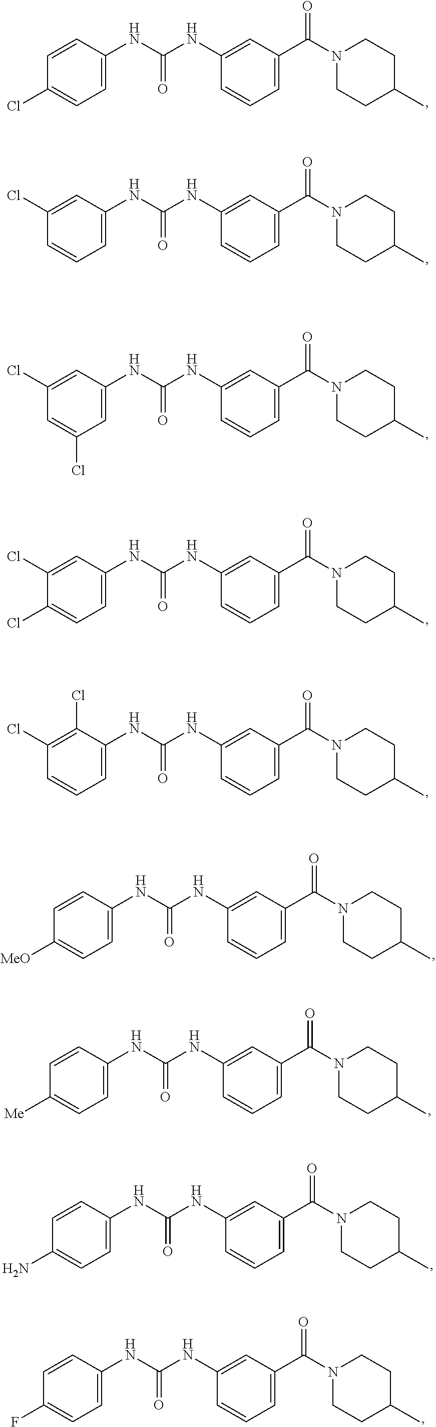

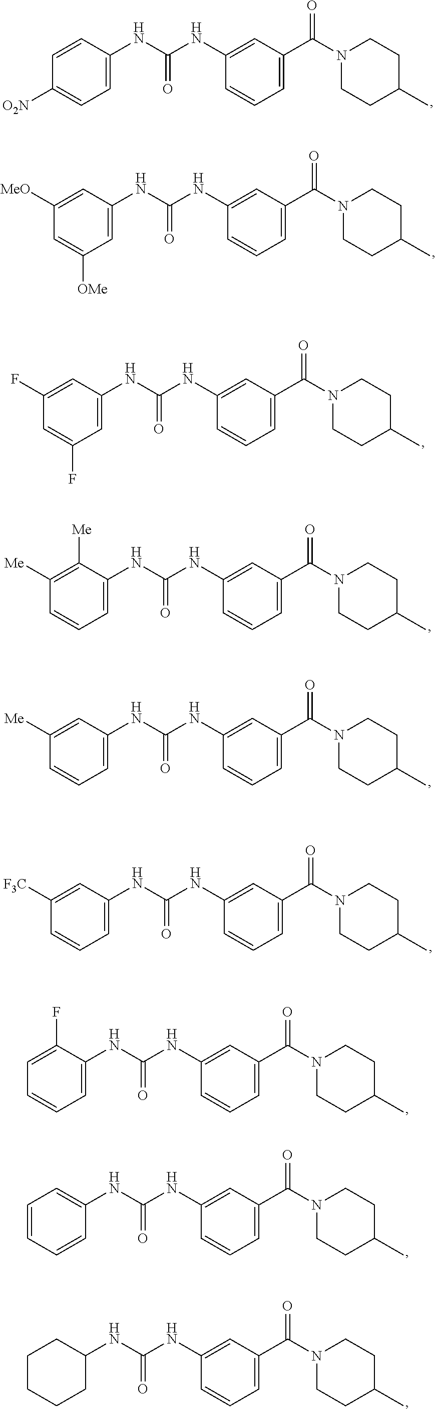

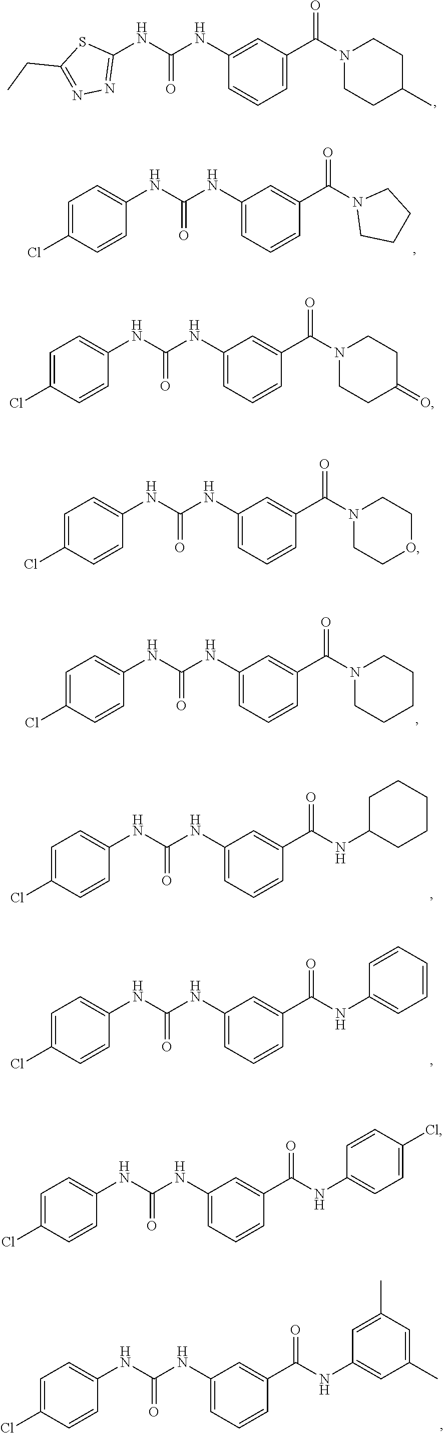

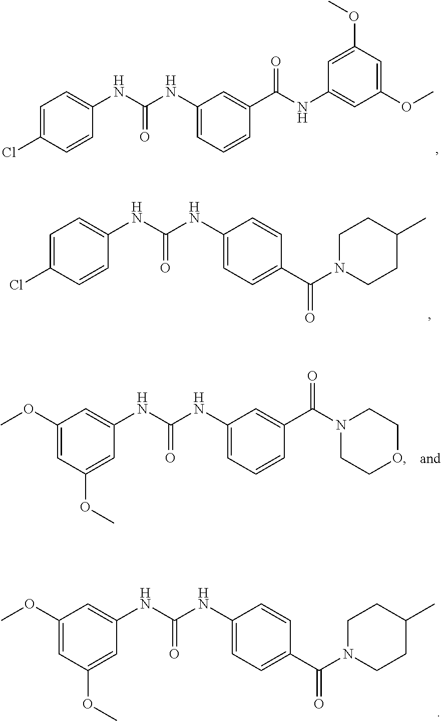

Clause 21. A compound, or a pharmaceutically acceptable salt thereof, wherein the compound is selected from the group consisting of:

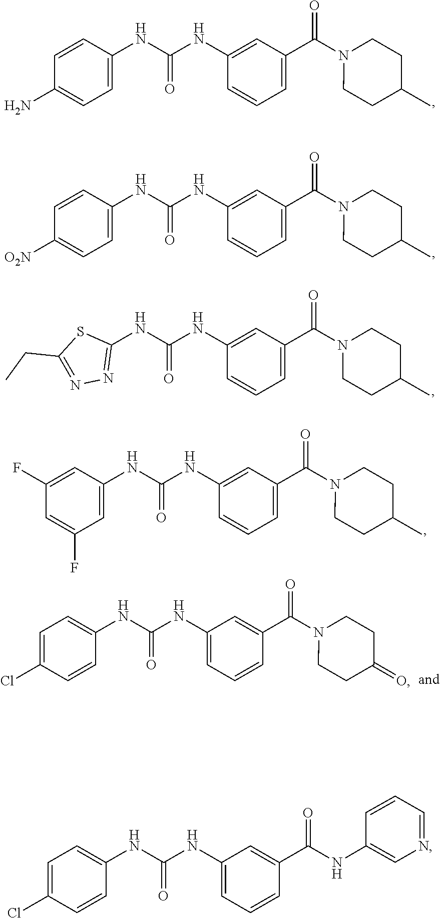

or a pharmaceutically acceptable salt thereof.

Clause 22. A pharmaceutical composition comprising a compound of formula (II), or a pharmaceutically acceptable salt thereof, and a pharmaceutically acceptable carrier,

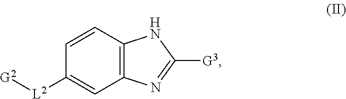

wherein:
G² is a 5- to 12-membered heteroaryl, a 6- to 12-membered aryl, C₃₋₁₀carbocyclyl, or a 4- to 12-membered heterocyclyl, wherein G² is optionally substituted with 1-3 R²;
L² is —(CH₂)₀₋₄—C(O)—(CH₂)₀₋₄—N(H)—;
G³ is a 5- to 12-membered heteroaryl, a 6- to 12-membered aryl, C₃₋₁₀carbocyclyl, or a 4- to 12-membered heterocyclyl, wherein G³ is optionally substituted with 1-3 R³;
R², at each occurrence, is independently halogen, cyano, C₁₋₆alkyl, C₁₋₆haloalkyl, —OR², —SR^{2a}, —CO₂R^{2a}, —C(O)R^{2a}, —SO₂R^{2b}, —N(R^{2b})₂, —CO₂N(R^{2b})₂, —NO₂, G^{2a}, —OG^{2a}, —SG^{2a}, or —N(R^{2b})-G^{2b};
R³, at each occurrence, is independently halogen, cyano, C₁₋₆alkyl, C₁₋₆haloalkyl, —OR^{3a}, —SR^{3a}, —CO₂R^{3a}, —C(O)R^{3a}, —SO₂R^{3b}, —N(R^{3b})₂, —CO₂N(R^{3b})₂, —NO₂, G^{3a}, —OG^{3a}, —SG^{3a}, or —N(R^{2b})-G^{2b};
R^{2a} and R^{3a}, at each occurrence, are each independently hydrogen or C₁₋₆alkyl;
R^{2b} and R^{3b}, at each occurrence, are each independently hydrogen or C₁₋₆alkyl;
G^{2a} and G^{3a}, at each occurrence, are each independently C₃₋₆carbocyclyl, phenyl, a 4- to 6-membered heterocyclyl, or a 5- to 6-membered heteroaryl, wherein G^{2a} and G^{3a} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl, and
G^{2b} and G^{3b}, at each occurrence, are each independently C₃₋₆carbocyclyl or phenyl, wherein G^{2b} and G^{3b} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl.

Clause 23. The pharmaceutical composition of clause 22, wherein L² is —N(H)—C(O)—.

Clause 24. The pharmaceutical composition of clause 22, wherein L² is —C(O)—(CH₂)₁₋₃—N(H)—.

Clause 25. The pharmaceutical composition of claim 22, wherein L² is —(CH₂)₁₋₃—C(O)—N(H)—.

Clause 26. The pharmaceutical composition of any one of claims 22-25, wherein G² is the 6- to 12-membered aryl.

Clause 27. The pharmaceutical composition of clause 26, where the 6- to 12-membered aryl at G² is phenyl.

Clause 28. The pharmaceutical composition of clause 27, wherein G² is

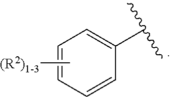

Clause 29. The pharmaceutical composition of any one of claims 22-25, wherein G² is the 5- to 12-membered heteroaryl.

Clause 30. The pharmaceutical composition of clause 29, wherein the 5- to 12-membered heteroaryl at G² is furanyl, thiophenyl, pyrrolyl, or imidazolyl.

Clause 31. The pharmaceutical composition of clause 30, wherein G² is

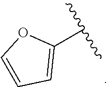 [SMILES: *c1ccco1]

Clause 32. The pharmaceutical composition of any one of claims 22-31, wherein R², at each occurrence, is independently selected from the group consisting of halogen, C₁₋₆alkyl, —OC₁₋₄alkyl, —NH₂, —OC₁₋₂fluoroalkyl, C₁₋₂fluoroalkyl, or —NO₂.

Clause 33. The pharmaceutical composition of any one of clauses 22-32, wherein G³ is the 5- to 12-membered heteroaryl.

Clause 34. The pharmaceutical composition of clause 33, wherein the 5- to 12-membered heteroaryl at G³ is thiophenyl, furanyl, pyrrolyl, or imidazolyl.

Clause 35. The pharmaceutical composition of clause 34, wherein G³ is

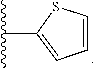 [SMILES: *c1cccs1]

Clause 36. The pharmaceutical composition of any one of clauses 22-32, wherein G³ is the 6- to 12-membered aryl.

Clause 37. The pharmaceutical composition of clause 36, wherein the 6- to 12-membered aryl at G³ is phenyl.

Clause 38. The pharmaceutical composition of clause 37, wherein G³ is

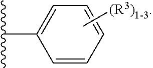

Clause 39. The pharmaceutical composition of any one of clauses 22-38, wherein R³, at each occurrence, is independently selected from the group consisting of halogen, C₁₋₆alkyl, —OC₁₋₄alkyl, —N—H₂, —OC₁₋₂fluoroalkyl, C₁₋₂fluoroalkyl, or —NO₂.

Clause 40. The pharmaceutical composition of clause 22, wherein the compound of formula (II) is selected from the group consisting of:

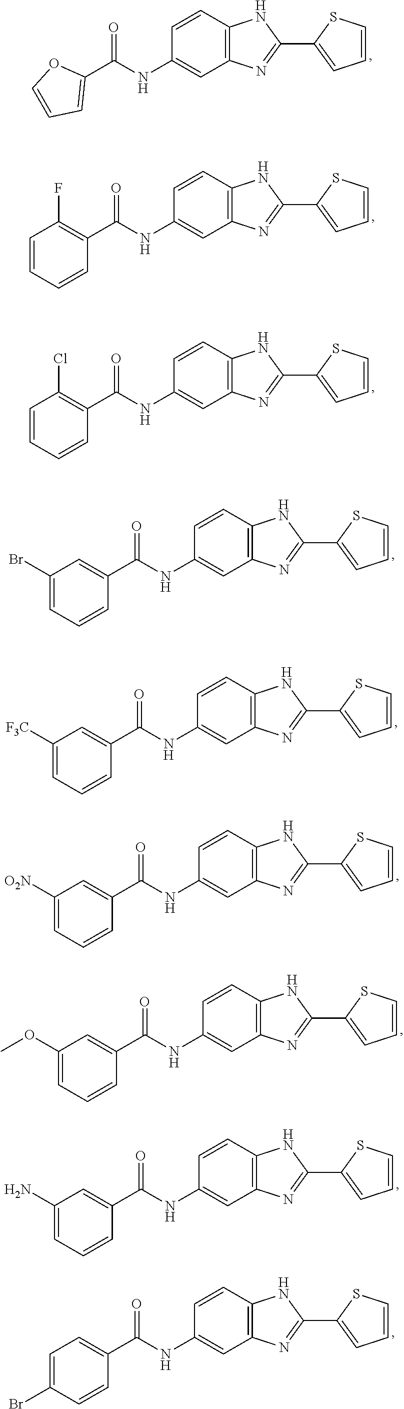

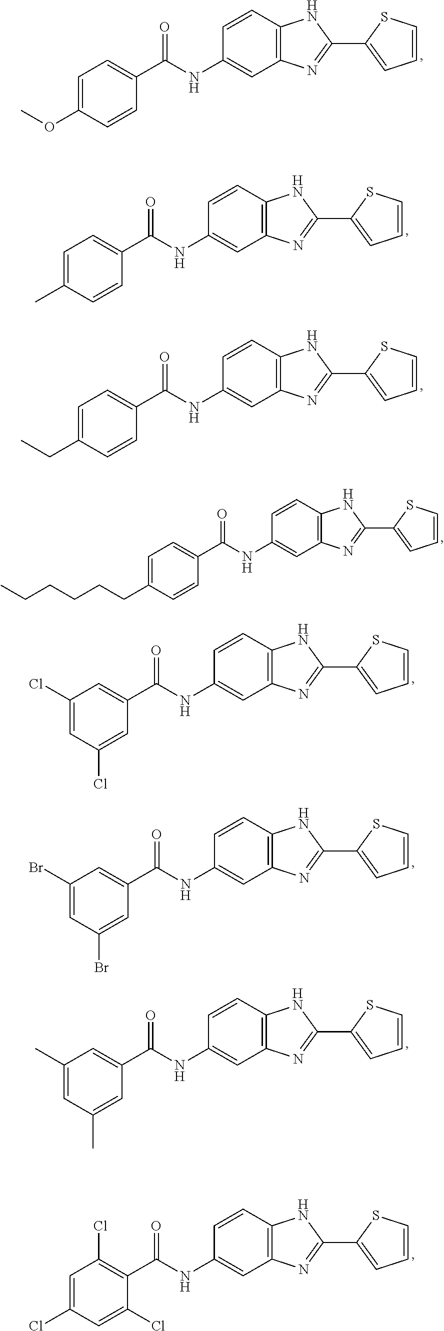

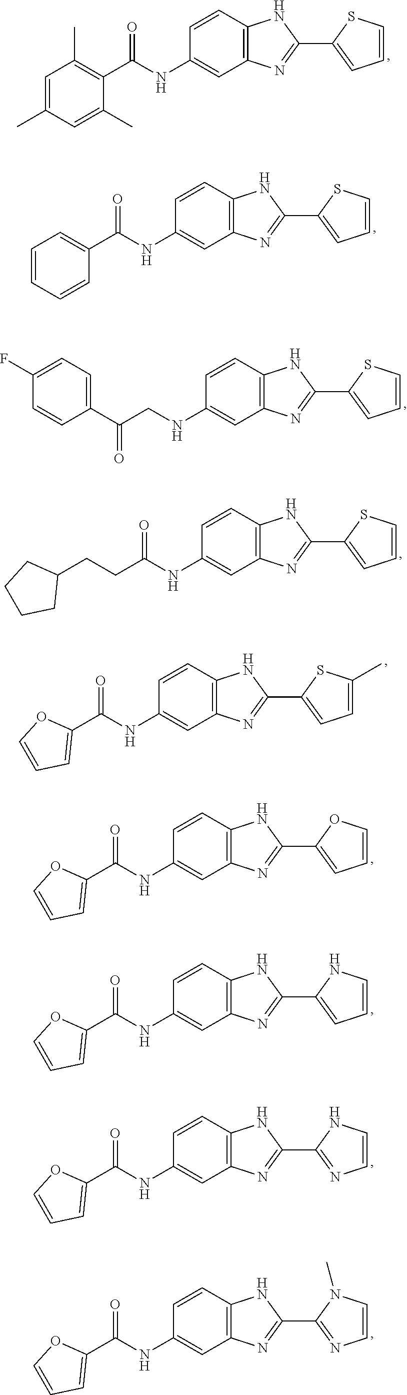

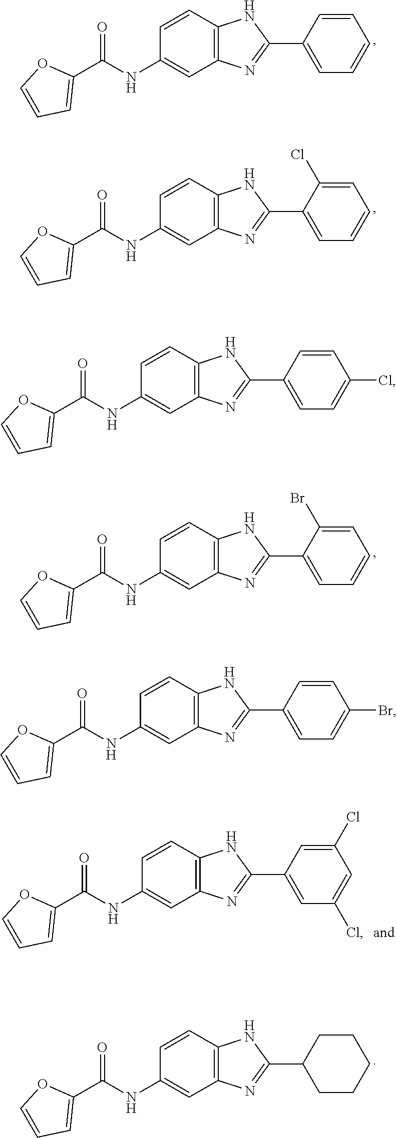

Clause 41. A compound, or a pharmaceutically acceptable salt thereof, wherein the compound is selected from the group consisting of:

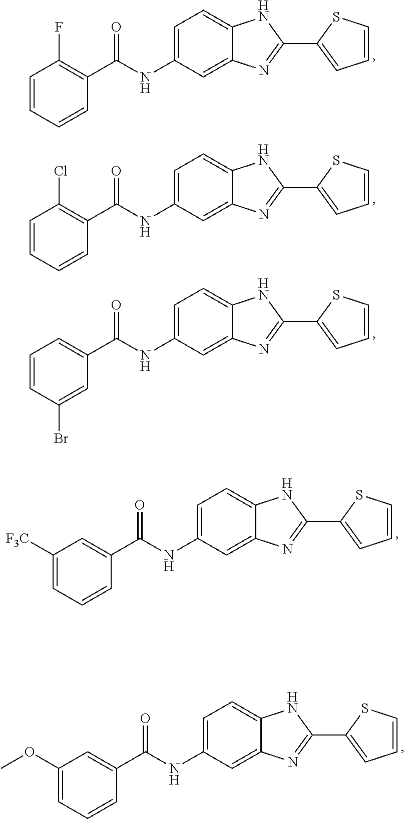

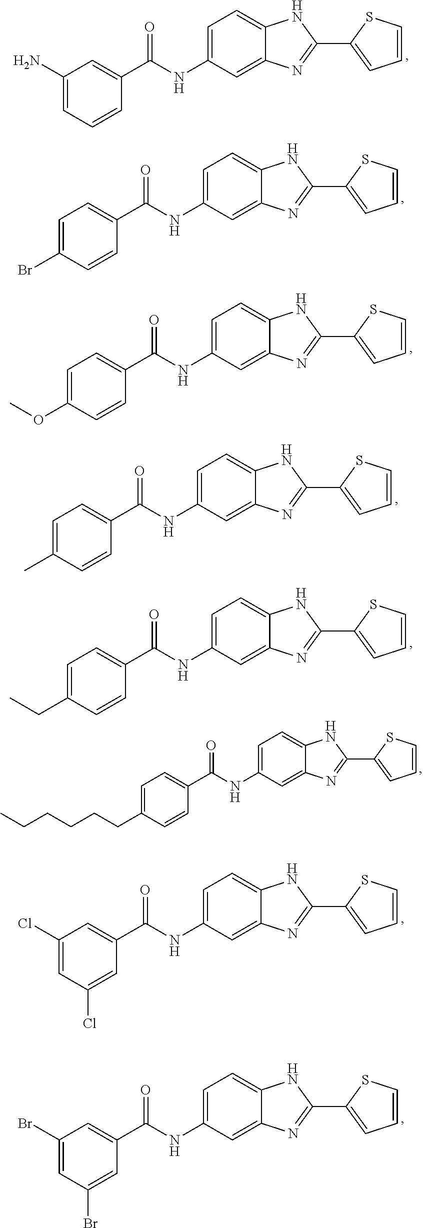

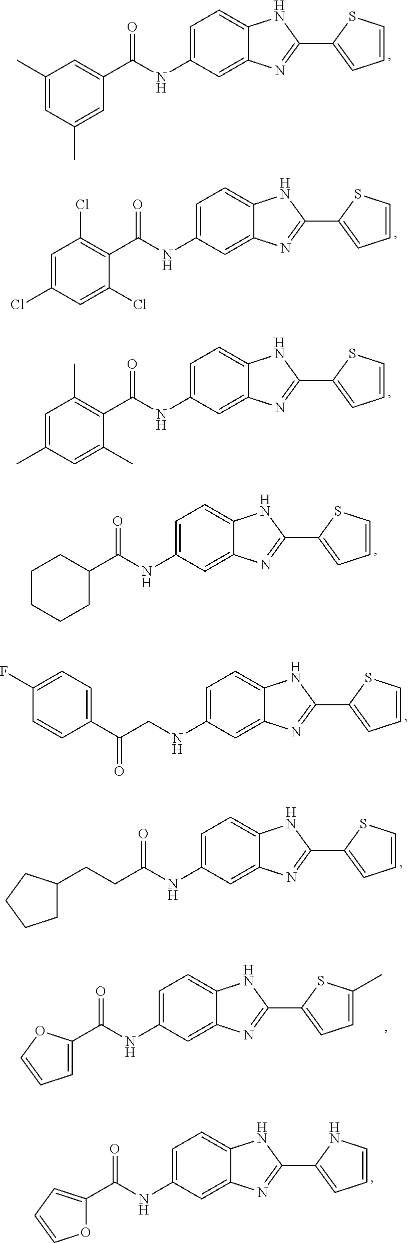

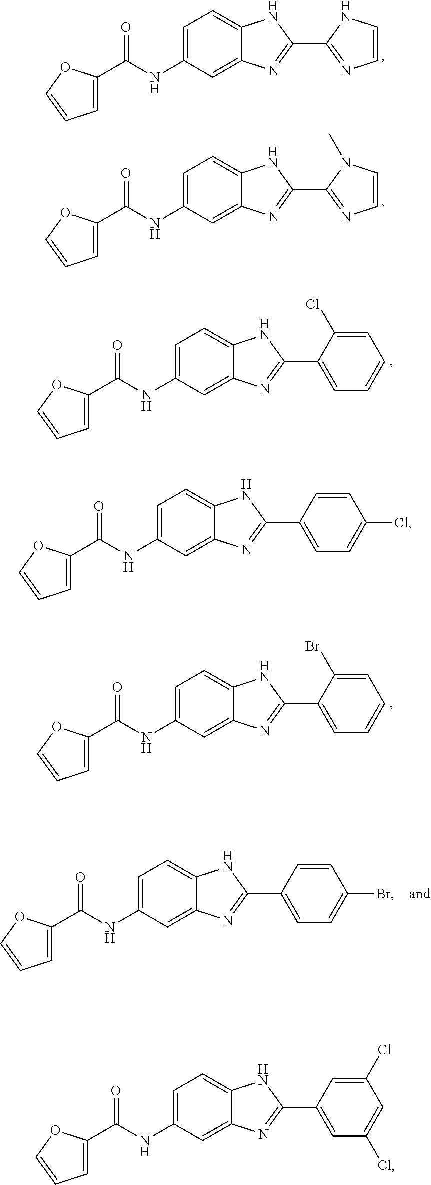

or a pharmaceutically acceptable salt thereof.

Clause 42. The pharmaceutical composition of any one of clauses 1-20 or any one of clauses 22-40, wherein the composition further comprises an antibiotic.

Clause 43. The pharmaceutical composition of clause 42, wherein the antibiotic comprises one or more selected from the group consisting of a cephalosporin, a carbapenem, an aminoglycoside, a fluoroquinolone, a glycopeptide, a lipoglycopeptide, a macrolide, a monobactams, an oxazolidinone, a penicillin, a polypeptide, a rifamycin, a sulfonamide, a streptogramin, and a tetracycline.

Clause 44. A method for treating a disease or disorder associated with a bacterial infection in a mammal, the method comprising administering to the mammal a therapeutically effective amount of the compound of clause 21 or clause 41, or pharmaceutically acceptable salt thereof, or the pharmaceutical composition of any one of clauses 1-20 or any one of clauses 22-40.

Clause 45. The method of clause 44, wherein the disease or disorder is associated with a Staphylococcus infection, penicillin binding protein 4 dysfunction, or a combination thereof.

Clause 46. The method of clause 45, wherein the Staphylococcus infection is the result of a methicillin resistant Staphylococcus aureus strain.

Clause 47. The method of clause 46, wherein the methicillin resistant Staphylococcus aureus strain is AH-1263, ATCC BAA-1556, AH-2204, BAA-811, 700789, BAA-1770, 43300, BAA-168, 33591, BAA-44, 700699, or a combination thereof.

Clause 48. The compound of clause 21 or clause 41, or pharmaceutically acceptable salt thereof, or the pharmaceutical composition of any one of clauses 1-20 or any one of clauses 22-40, for use in the treatment of a disease or disorder associated with a bacterial infection in a mammal.

Clause 49. The use of the compound of clause 21 or clause 41, or pharmaceutically acceptable salt thereof, or the pharmaceutical composition of any one of clauses 1-20 or any one of clauses 22-40, for the preparation of a medicament for the treatment of a disease or disorder associated with a bacterial infection in a mammal.

1. A pharmaceutical composition comprising a compound of formula (I), or a pharmaceutically acceptable salt thereof, and a pharmaceutically acceptable carrier, 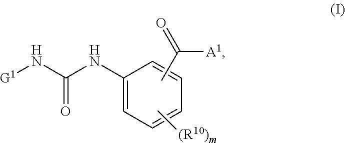
wherein:
A¹ is 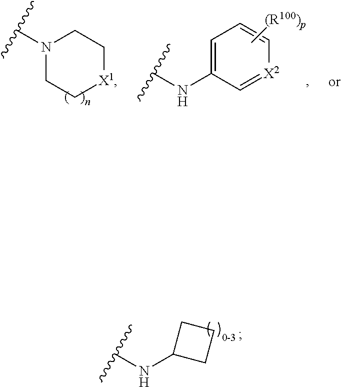
G¹ is a 6- to 12-membered aryl, C₃₋₁₀carbocyclyl, a 5- to 12-membered heteroaryl, or a 4- to 12-membered heterocyclyl, wherein G¹ is optionally substituted with 1-3 R¹;
X¹ is CHR^{X}, C═O, or O;
X² is CH or N;
R^{X}, at each occurrence, is C₁₋₆alkyl, hydrogen, C₁₋₄haloalkyl, halogen, or cyano;
R¹, at each occurrence, is independently halogen, cyano, C₁₋₆alkyl, C₁₋₆haloalkyl, —OR^{1a}, —SR^{1a}, —CO₂R^{1a}, —C(O)R^{1a}, —SO₂R^{1b}, —N(R^{1b})₂, —CO₂N(R^{1b})₂, —NO₂, G^{1a}, —OG^{1a}, —SG^{1a}, —N(R^{1b})-G^{1b}, -L¹-Y¹, —O-L¹-Y¹, —S-L¹-Y¹, or —N(R^{1b})-L¹-Y¹;
L¹, at each occurrence, is independently a C₁₋₆alkylene, wherein optionally 1 or 2 methylene groups in the alkylene of L¹ are independently replaced with —O—, —S—, —SO₂—, —C(O)—, or —N(R^{1b})—, wherein 2 methylene groups replaced with —O—, —S—, —SO₂—, or N(R^{1b})— are separated by two or more carbon atoms in the alkylene;
Y¹, at each occurrence, is independently hydrogen, halogen, cyano, C₁₋₆alkyl, C₁₋₆haloalkyl, —OR^{1a}, —SR^{1a}, —CO₂R^{1a}, —C(O)R^{1a}, —SO₂R^{1b}, —N(R^{1b})₂, —CO₂N(R^{1b})₂, G^{1a}, or —OG^{1a};
R^{1a}, at each occurrence, is independently hydrogen, C₁₋₆alkyl, or C₁₋₂haloalkyl;
R^{1b}, at each occurrence, is independently hydrogen or C₁₋₆alkyl;
G^{1a}, at each occurrence, is independently C₃₋₆carbocyclyl, phenyl, a 4- to 6-membered heterocyclyl, or a 5- to 6-membered heteroaryl, wherein G^{1a} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
G^{1b}, at each occurrence, is independently C₃₋₆carbocyclyl or phenyl, wherein G^{1b} is optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl;
R¹⁰, at each occurrence, is independently halogen, C₁₋₄alkyl, —OC₁₋₄alkyl, C₁₋₂haloalkyl, cyano, or —OC₁₋₂haloalkyl;
R¹⁰⁰, at each occurrence, is independently halogen, C₁₋₄alkyl, —OC₁₋₄alkyl, C₁₋₂haloalkyl, cyano, or —OC₁₋₂haloalkyl;
m is 0, 1, 2, 3, or 4;
n is 0, 1, or 2; and
p is 0, 1, 2, or 3.

2. The pharmaceutical composition of claim 1, wherein the compound of formula (I) is a compound of formula (I-a):

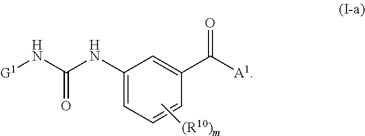

3. The pharmaceutical composition of claim 1, wherein G¹ is the 6- to 12-membered aryl.

4. The pharmaceutical composition of claim 3, wherein the 6- to 12-membered aryl at G¹ is phenyl.

5. The pharmaceutical composition of claim 1, wherein G¹ is substituted with 1-2 substituents selected from the group consisting of halogen, C₁₋₆alkyl, —OC₁₋₄alkyl, —NH₂, —OC₁₋₂fluoroalkyl, C₁₋₂fluoroalkyl, or —NO₂.

6. The pharmaceutical composition of claim 1, wherein G¹ is

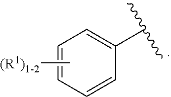

7. The pharmaceutical composition of claim 1, wherein m is 0.

8. The pharmaceutical composition of claim 1, wherein A¹ is

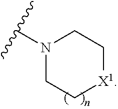

9. The pharmaceutical composition of claim 8, wherein n is 1.

10. The pharmaceutical composition of claim 8, wherein X¹ is CHR^{X}.

11. The pharmaceutical composition of claim 10, wherein R^{X} is C₁₋₆alkyl.

12. The pharmaceutical composition of claim 11, wherein R^{X} is methyl.

13. The pharmaceutical composition of claim 8, wherein X¹ is C═O.

14. The pharmaceutical composition of claim 8, wherein X¹ is O.

15. The pharmaceutical composition of claim 1, wherein A¹ is

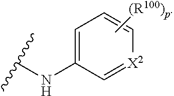

16. The pharmaceutical composition of claim 15, wherein X² is CH.

17. The pharmaceutical composition of claim 15, wherein p is 1 or 2.

18. The pharmaceutical composition of claim 15, wherein R¹⁰⁰ is F, Cl, —CH₃, or —OCH₃.

19. The pharmaceutical composition of claim 15, wherein X² is N.

20. The pharmaceutical composition of claim 1, wherein the compound of formula (I) is selected from the group consisting of:

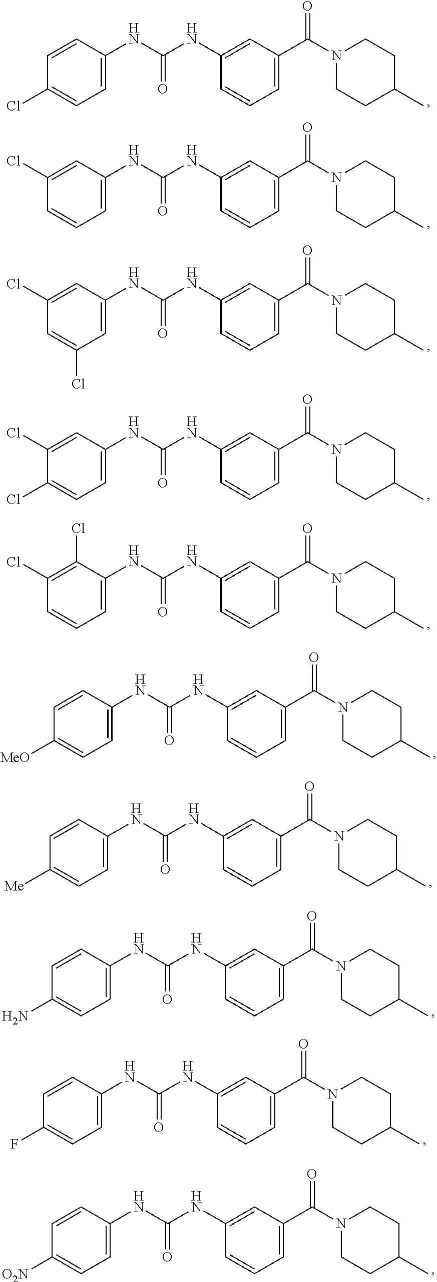

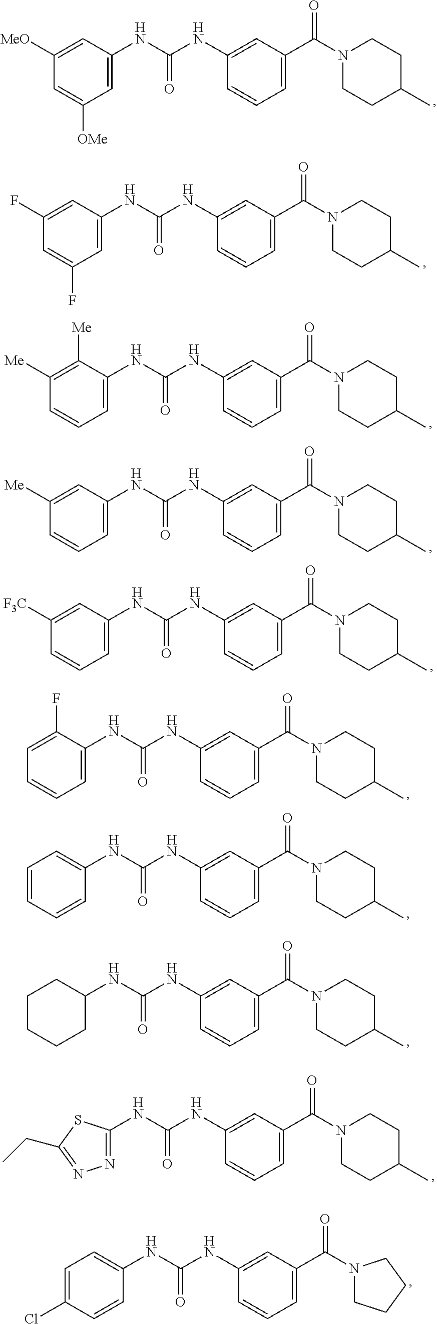

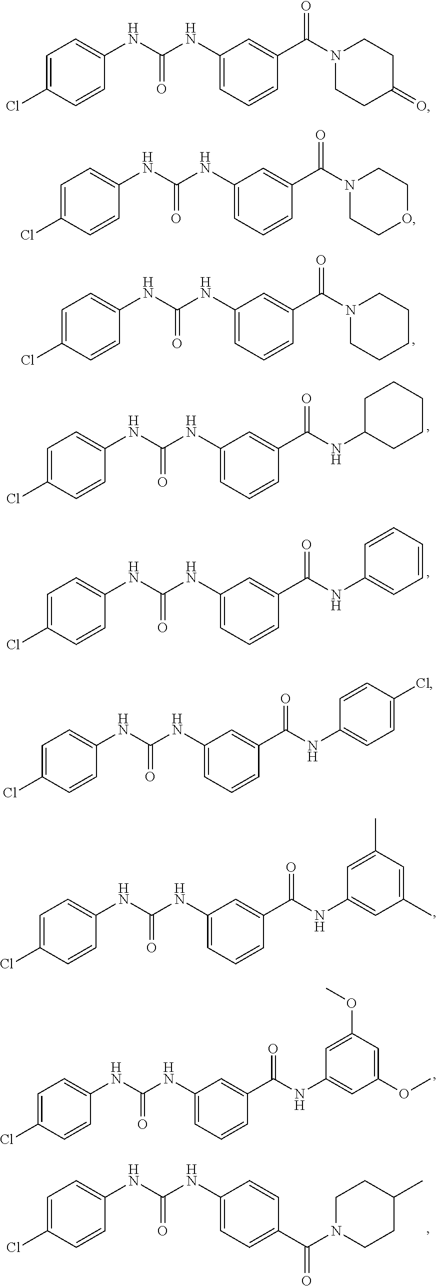

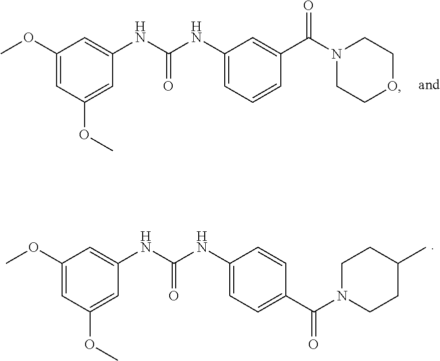

21. A compound, or a pharmaceutically acceptable salt thereof, wherein the compound is selected from the group consisting of: 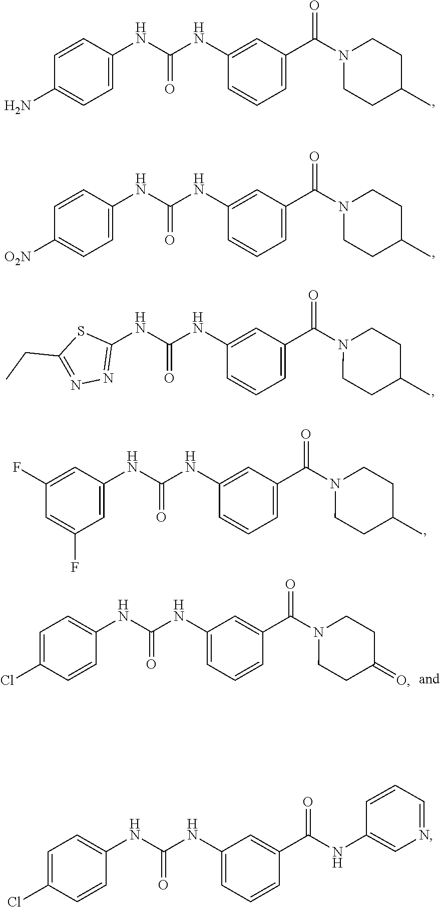
or a pharmaceutically acceptable salt thereof.

22. A pharmaceutical composition comprising a compound of formula (II), or a pharmaceutically acceptable salt thereof, and a pharmaceutically acceptable carrier, 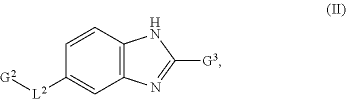
wherein:
G² is a 5- to 12-membered heteroaryl, a 6- to 12-membered aryl, C₃₋₁₀carbocyclyl, or a 4- to 12-membered heterocyclyl, wherein G² is optionally substituted with 1-3 R²;
L² is —(CH₂)₀₋₄—C(O)—(CH₂)₀₋₄—N(H)—;
G³ is a 5- to 12-membered heteroaryl, a 6- to 12-membered aryl, C₃₋₁₀carbocyclyl, or a 4- to 12-membered heterocyclyl, wherein G³ is optionally substituted with 1-3 R³;
R², at each occurrence, is independently halogen, cyano, C₁₋₆alkyl, C₁₋₆haloalkyl, —OR^{2a}, —SR^{2a}, —CO₂R^{2a}, —C(O)R^{2a}, —SO₂R^{2b}, —N(R^{2b})₂, —CO₂N(R^{2b})₂, —NO₂, G^{2a}, —OG^{2a}, —SG^{2a}, or —N(R^{2b})-G^{2b};
R³, at each occurrence, is independently halogen, cyano, C₁₋₆alkyl, C₁₋₆haloalkyl, —OR^{3a}, —SR^{3a}, —CO₂R^{3a}, —C(O)R^{3a}, —SO₂R^{3b}, —N(R^{3b})₂, —CO₂N(R^{3b})₂, —NO₂, G^{3a}, —OG^{3a}, —SG^{3a}, or —N(R^{2b})-G^{2b},
R^{2a} and R^{3a}, at each occurrence, are each independently hydrogen or C₁₋₆alkyl;
R^{2b} and R^{3b}, at each occurrence, are each independently hydrogen or C₁₋₆alkyl;
G^{2a} and G^{3a}, at each occurrence, are each independently C₃₋₆carbocyclyl, phenyl, a 4- to 6-membered heterocyclyl, or a 5- to 6-membered heteroaryl, wherein G^{2a} and G^{3a} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl; and
G^{2b} and G^{3b}, at each occurrence, are each independently C₃₋₆carbocyclyl or phenyl, wherein G^{2b} and G^{3b} are each optionally substituted with 1-4 substituents independently selected from the group consisting of C₁₋₄alkyl, C₁₋₂haloalkyl, halogen, cyano, —OC₁₋₄alkyl, and —OC₁₋₂haloalkyl.

23. The pharmaceutical composition of claim 22, wherein L² is —N(H)—C(O)—.

24. The pharmaceutical composition of claim 22, wherein L² is —C(O)—(CH₂)₁₋₃—N(H)—.

25. The pharmaceutical composition of claim 22, wherein L² is —(CH₂)₁₋₃—C(O)—N(H)—.

26. The pharmaceutical composition of claim 22, wherein G² is the 6- to 12-membered aryl.

27. The pharmaceutical composition of claim 26, where the 6- to 12-membered aryl at G² is phenyl.

28. The pharmaceutical composition of claim 27, wherein G² is

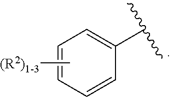

29. The pharmaceutical composition of claim 22, wherein G² is the 5- to 12-membered heteroaryl.

30. The pharmaceutical composition of claim 29, wherein the 5- to 12-membered heteroaryl at G² is furanyl, thiophenyl, pyrrolyl, or imidazolyl.

31. The pharmaceutical composition of claim 30, wherein G² is

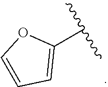 [SMILES: *c1ccco1]

32. The pharmaceutical composition of claim 22, wherein R², at each occurrence, is independently selected from the group consisting of halogen, C₁₋₆alkyl, —OC₁₋₄alkyl, —NH₂, —OC₁₋₂fluoroalkyl, C₁₋₂fluoroalkyl, or —NO₂.

33. The pharmaceutical composition of claim 22, wherein G³ is the 5- to 12-membered heteroaryl.

34. The pharmaceutical composition of claim 33, wherein the 5- to 12-membered heteroaryl at G³ is thiophenyl, furanyl, pyrrolyl, or imidazolyl.

35. The pharmaceutical composition of claim 34, wherein G³ is

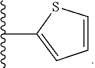 [SMILES: *c1cccs1]

36. The pharmaceutical composition of claim 22, wherein G³ is the 6- to 12-membered aryl.

37. The pharmaceutical composition of claim 36, wherein the 6- to 12-membered aryl at G³ is phenyl.

38. The pharmaceutical composition of claim 37, wherein G³ is

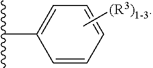

39. The pharmaceutical composition of claim 22, wherein R³, at each occurrence, is independently selected from the group consisting of halogen, C₁₋₆alkyl, —OC₁₋₄alkyl, —NH₂, —OC₁₋₂fluoroalkyl, C₁₋₂fluoroalkyl, or —NO₂.

40. The pharmaceutical composition of claim 22, wherein the compound of formula (II) is selected from the group consisting of:

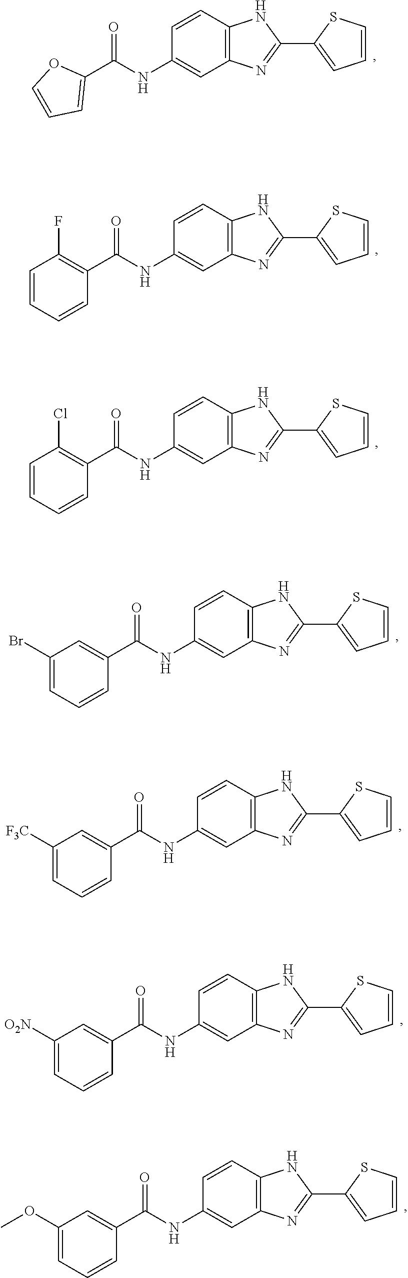

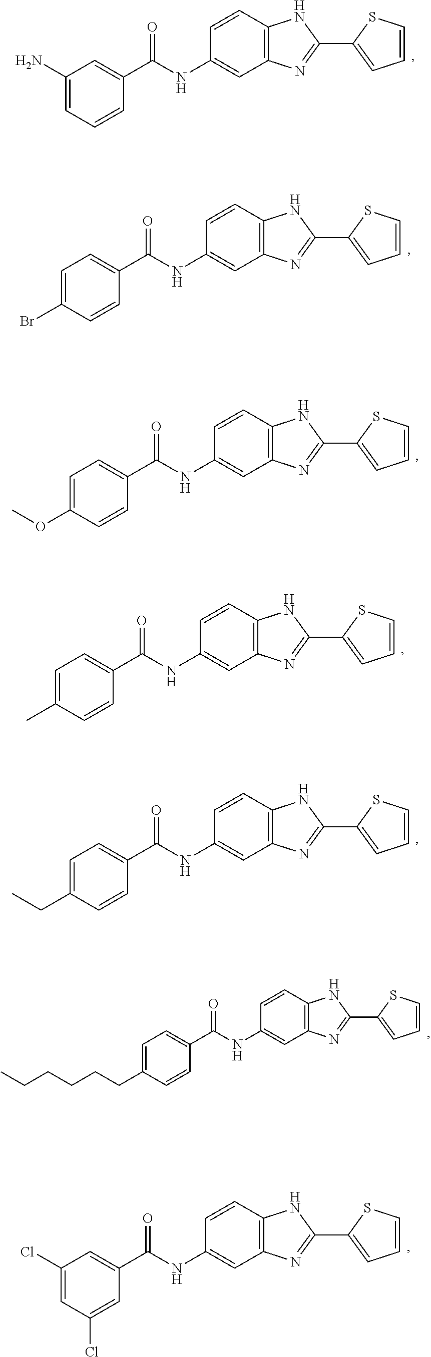

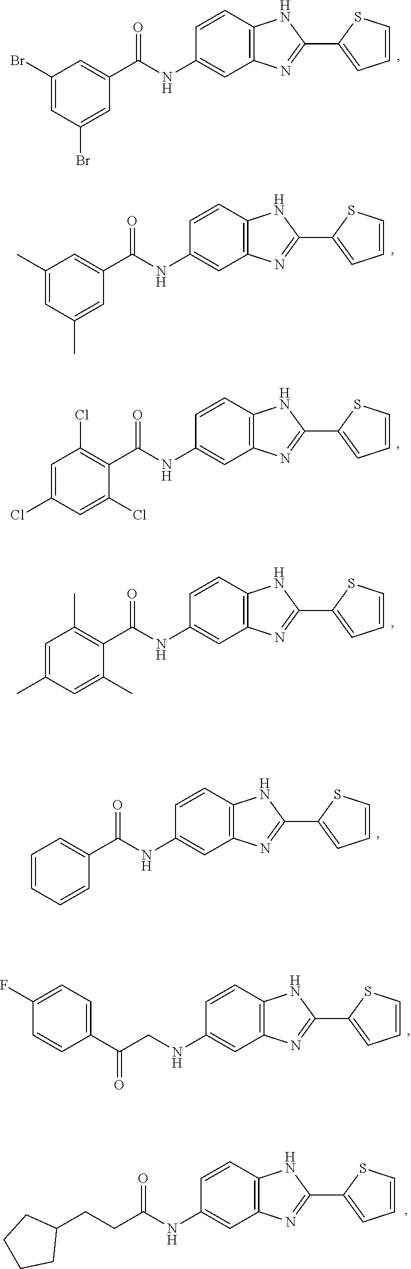

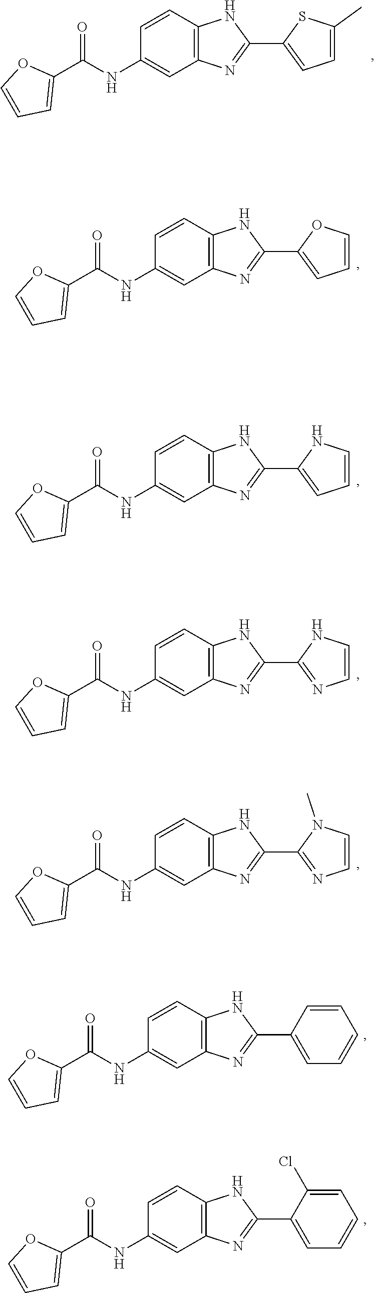

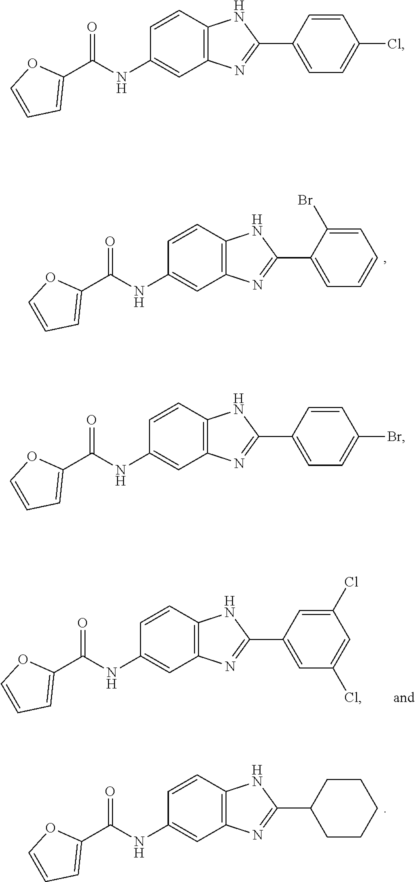 [SMILES: O=C(Nc1ccc2[nH]c(-c3ccc(Cl)cc3)nc2c1)c1ccco1 ; O=C(Nc1ccc2[nH]c(-c3ccccc3Br)nc2c1)c1ccco1 ; O=C(Nc1ccc2[nH]c(-c3ccc(Br)cc3)nc2c1)c1ccco1 ; O=C(Nc1ccc2[nH]c(-c3cc(Cl)cc(Cl)c3)nc2c1)c1ccco1 ; O=C(Nc1ccc2[nH]c(C3CCCCC3)nc2c1)c1ccco1]

41. A compound, or a pharmaceutically acceptable salt thereof, wherein the compound is selected from the group consisting of: 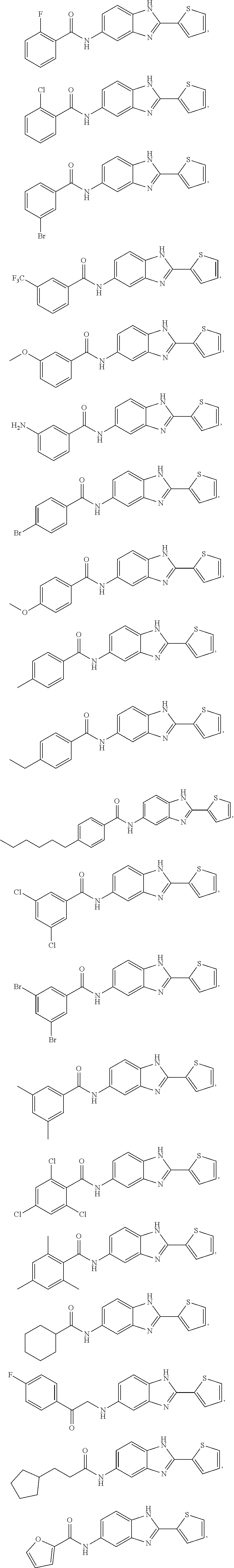 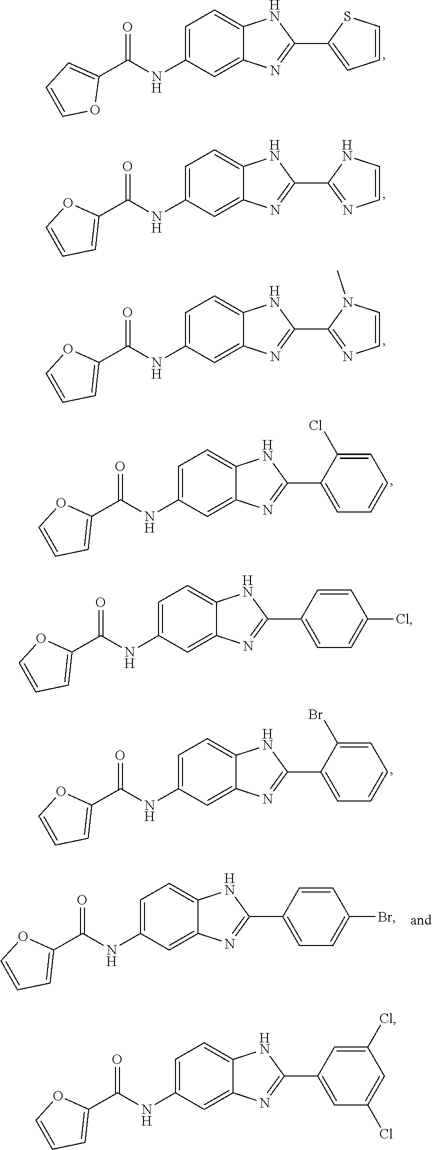
or a pharmaceutically acceptable salt thereof.

42. The pharmaceutical composition of claim 1 or claim 22, wherein the composition further comprises an antibiotic.

43. The pharmaceutical composition of claim 42, wherein the antibiotic comprises one or more selected from the group consisting of a cephalosporin, a carbapenem, an aminoglycoside, a fluoroquinolone, a glycopeptide, a lipoglycopeptide, a macrolide, a monobactams, an oxazolidinone, a penicillin, a polypeptide, a rifamycin, a sulfonamide, a streptogramin, and a tetracycline.

44. A method for treating a disease or disorder associated with a bacterial infection in a mammal, the method comprising administering to the mammal a therapeutically effective amount of the compound of claim 21 or claim 41, or pharmaceutically acceptable salt thereof, or the pharmaceutical composition of claim 1 or claim 22.

45. The method of claim 44, wherein the disease or disorder is associated with a Staphylococcus infection, penicillin binding protein 4 dysfunction, or a combination thereof.

46. The method of claim 45, wherein the Staphylococcus infection is the result of a methicillin resistant Staphylococcus aureus strain.

47. The method of claim 46, wherein the methicillin resistant Staphylococcus aureus strain is AH-1263, ATCC BAA-1556, AH-2204, BAA-811, 700789, BAA-1770, 43300, BAA-168, 33591, BAA-44, 700699, or a combination thereof.

48. The compound of claim 21 or 41, or pharmaceutically acceptable salt thereof, or the pharmaceutical composition of claim 1 or claim 22, for use in the treatment of a disease or disorder associated with a bacterial infection in a mammal.

49. The use of the compound of claim 21 or 41, or pharmaceutically acceptable salt thereof, or the pharmaceutical composition of claim 1 or claim 22, for the preparation of a medicament for the treatment of a disease or disorder associated with a bacterial infection in a mammal.
